SEQUENCE LISTING (163 sequences)

SEQ ID NO: 1 - protein, 450 aa, Artificial Sequence
  Description of Artificial Sequence Synthetic polypeptide
QMQLVQSGAEVKKTGSSVKVSCKASGFNIKDYYLHWVRQAPGQALEWMGWIDPDQGDTEYAQKFQDRVTITRDRSMSTAYMELSSLRSEDTAMYYCNAAYGSSSYPMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 214 aa, Artificial Sequence
  Description of Artificial Sequence Synthetic polypeptide
NIQMTQSPSAMSASVGDRVTITCKASQDIHRYLSWFQQKPGKVPKHLIYRANRLVSGVPSRFSGSGSGTEFTLTISSLQPEDFATYYCLQYDEFPYTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 490 aa, Artificial Sequence
  Description of Artificial Sequence Synthetic polypeptide
DIQMTQSPSSLSASVGDRVTITCRSTKSLLHSNGITYLYWYQQKPGKAPKLLIYQMSNLASGVPSRFSSSGSGTDFTLTISSLQPEDFATYYCAQNLEIPRTFGQGTKVELKRATPSHNSHQVPSAGGPTANSGTSGSEVQLVQSGPGLVQPGGSVRISCAASGYTFTNYGMNWVKQAPGKGLEWMGWINTYTGESTYADSFKGRFTFSLDTSASAAYLQINSLRAEDTAVYYCARFAIKGDYWGQGTLLTVSSGGSGGSGGASKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 4 - protein, 446 aa, Artificial Sequence
  Description of Artificial Sequence Synthetic polypeptide
EVQLLESGGGLVQPGGSLRLSCAASGFTFSNFAMSWVRQAPGKGLEWVSTISASGGRTFYADSVKGRITISRDNSKNTLFLQMNGLRAEDTAVYYCAKEGSFGEGVDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 5 - protein, 119 aa, Artificial Sequence
  Description of Artificial Sequence Synthetic polypeptide
EVQLLESGGGLVQPGGSLRLSCAASGFTFSNFAMSWVRQAPGKGLEWVSTISASGGRTFYADSVKGRITISRDNSKNTLFLQMNGLRAEDTAVYYCAKEGSFGEGVDPWGQGTLVTVSS

SEQ ID NO: 6 - protein, 214 aa, Artificial Sequence
  Description of Artificial Sequence Synthetic polypeptide
SYEMTQPPSVSVSPGQTARITCSGDALPKKYAYWYQQKSGQAPVLVIYEDNKRPSGIPERFSGSSSGTMATLTISGAQVEDEADYYCYSTDISGNHWVFGGGTELTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 7 - protein, 449 aa, Artificial Sequence
  Description of Artificial Sequence Synthetic polypeptide
EVQLLESGGGLVQPGGSLRLSCAASGFTFSNFAMSWVRQAPGKGLEWVSTISASGGRTFYADSVKGRITISRDNSKNTLFLQMNGLRAEDTAVYYCAKEGSFGEGVDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 214 aa, Artificial Sequence; identical to SEQ ID NO: 6
  Description of Artificial Sequence Synthetic polypeptide

SEQ ID NO: 9 - protein, 449 aa, Artificial Sequence
  Description of Artificial Sequence Synthetic polypeptide
EVQLLESGGGLVQPGGSLRLSCAASGFTFSNFAMSWVRQAPGKGLEWVSTISASGGRTFYADSVKGRITISRDNSKNTLYLQMNSLRAEDTAVYYCAKEGSFGEGVDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 119 aa, Artificial Sequence
  Description of Artificial Sequence Synthetic polypeptide
EVQLLESGGGLVQPGGSLRLSCAASGFTFSNFAMSWVRQAPGKGLEWVSTISASGGRTFYADSVKGRITISRDNSKNTLYLQMNSLRAEDTAVYYCAKEGSFGEGVDPWGQGTLVTVSS

SEQ ID NO: 11 - protein, 214 aa, Artificial Sequence
  Description of Artificial Sequence Synthetic polypeptide
SYEMTQPPSVSVSPGQTARITCSGDALPKKYAYWYQQKSGQAPVLVIYEDNKRPSGIPERFSGSSSGTMATLTISGAQVEDEADYYCYSTDISGNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 12 - protein, 449 aa, Artificial Sequence
  Description of Artificial Sequence Synthetic polypeptide
EVQLLESGGGLVQPGGSLRLSCAASGFTFSNFAMSWVRQAPGKGLEWVSTISASGGRTFYADSVKGRFTISRDNSKNTLYLQMNSLRAEDTAVYYCAKEGSFGEGVDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - protein, 119 aa, Artificial Sequence
  Description of Artificial Sequence Synthetic polypeptide
EVQLLESGGGLVQPGGSLRLSCAASGFTFSNFAMSWVRQAPGKGLEWVSTISASGGRTFYADSVKGRFTISRDNSKNTLYLQMNSLRAEDTAVYYCAKEGSFGEGVDPWGQGTLVTVSS

SEQ ID NO: 14 - protein, 214 aa, Artificial Sequence
  Description of Artificial Sequence Synthetic polypeptide
SYELTQPPSVSVSPGQTARITCSGDALPKKYAYWYQQKSGQAPVLVIYEDNKRPSGIPERFSGSSSGTMATLTISGAQVEDEADYYCYSTDISGNHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 15 - protein, 5 aa, Artificial Sequence
  Description of Artificial Sequence Synthetic peptide
NFAMS

SEQ ID NO: 16 - protein, 17 aa, Artificial Sequence
  Description of Artificial Sequence Synthetic peptide
TISASGGRTFYADSVKG

SEQ ID NO: 17 - protein, 10 aa, Artificial Sequence
  Description of Artificial Sequence Synthetic peptide
EGSFGEGVDP

SEQ ID NO: 18 - protein, 11 aa, Artificial Sequence
  Description of Artificial Sequence Synthetic peptide
SGDALPKKYAY

SEQ ID NO: 19 - protein, 7 aa, Artificial Sequence
  Description of Artificial Sequence Synthetic peptide
EDNKRPS

SEQ ID NO: 20 - protein, 11 aa, Artificial Sequence
  Description of Artificial Sequence Synthetic peptide
YSTDISGNHWV

SEQ ID NO: 21 - protein, 123 aa, Artificial Sequence
  Description of Artificial Sequence Synthetic polypeptide
QVQLQQSGPELVKPGTSVKLSCRASGYTFTSYDINWVKQRPGQGLEWIGWIYPRDGSTKYNERFKGKATLTVDTSSSTAYMEFHTLTSEDSAVYFCARDPYYYGTIYGYFDVWGTGTTVTVSS

SEQ ID NO: 22 - protein, 119 aa, Artificial Sequence
  Description of Artificial Sequence Synthetic polypeptide
EVQLVESGGGLVQPKGSLKLSCAASGFTFNTYAMHWVRQAPGKGLEWVARIRSKTYNYATFYADSVKDRFTISRDDSQSMLYLQMNNLKTEDTAMFYCVTGSSYAMDYWGQGTSVTVSS

SEQ ID NO: 23 - protein, 120 aa, Artificial Sequence
  Description of Artificial Sequence Synthetic polypeptide
QVQLQQPGAELVRPGTSVKLSCKASGYTFTSYWMHWIKQRPGQGLEWIGVIDPSNSYSNYNQKFKGRATLTVDTYSSTAYMQLSSLTSEDSAVYYCARPYNHGRDAMDYWGQGTSVTVSS

SEQ ID NO: 24 - protein, 117 aa, Artificial Sequence
  Description of Artificial Sequence Synthetic polypeptide
QVQLQQSGAELARPGASVQLSCKASGYTFTSYGISWLKQRAGQGLEWIGEIYPRSDNTYYNEKFKGKATLTADKSSSTAYMELRSLTSEDSAVYFCARRAGMWFAYWGQGTLVTVSA

SEQ ID NO: 25 - protein, 120 aa, Artificial Sequence
  Description of Artificial Sequence Synthetic polypeptide
DVQLQESGPGLVKPSQSLSLTCTVTGYSITSDFAWNWIRQFPGNKLEWVGYITYSGTTSYNPSLKSRISITRDTSKNQFFLQLSSVTTEDTATYYCARSFHFFAYGFAYWGQGTLVTVSA

SEQ ID NO: 26 - protein, 116 aa, Artificial Sequence
  Description of Artificial Sequence Synthetic polypeptide
QVQLQQSGAELARPGASVKVSCKASGYTFTSYGISWVKQRTGQGLEWIGEIYPTRDYTYYNENFKGKATLTADKSSTTAYMELRSLTSEDSAVYFCARDGPPFAYWGQGTLVTVSA

SEQ ID NO: 27 - protein, 113 aa, Artificial Sequence
  Description of Artificial Sequence Synthetic polypeptide
EVQLQQSGPELVKPGASVKISCKASGYTFTDYYMSWVKQSHGRSLEWIGEINPDNGDTSHNQKFKGKATMTVDKSSGTGNMEIRSLTSEDAGIYYCTSYFSYWGQGTLVTVSA

SEQ ID NO: 28 - protein, 115 aa, Artificial Sequence
  Description of Artificial Sequence Synthetic polypeptide
EVLLQQSVADLVRPGASVKLSCTASGLNIKNTYIHWVKQMPEQGLEWIGRIGPANGNTKYAPKFQGKATITADTSSDTAYLQLSSLTSEDTAIYYCALAYYRIYWGQGTTLTVSS

SEQ ID NO: 29 - protein, 115 aa, Artificial Sequence
  Description of Artificial Sequence Synthetic polypeptide
EVQLQQSVAELVRPGASVKLSCTASGFNIKNTYMHWVKQRPEQGLEWIGRIAPANGNTKYAPKFQDKATITANTSSNTAYLQLGSLTSEDTAIYYCAFIYQLPYWGQGTTLTVSS

SEQ ID NO: 30 - protein, 117 aa, Artificial Sequence
  Description of Artificial Sequence Synthetic polypeptide
QVQVQQSGPELVKPGTSVKISCKTSGYTFTDYYITWVKQRPGQGLEWIGWIFPGRGISFYNENFKDKATLTVDKSSNTAFMLLSSLTSEDSAVYFCARWGGTWFAYWGQGTLITVSA

SEQ ID NO: 31 - protein, 115 aa, Artificial Sequence
  Description of Artificial Sequence Synthetic polypeptide
EVQLQQSVAELVRPGASVKLSCTASGFNIKNTYMHWVKQRPEQGLEWIGRIAPANGNTKYDPKFQDKATITADSSSNTAYLHLSSLTSEDTAIYYCAFIYQLPYWGQGSTLTVSS

SEQ ID NO: 32 - protein, 116 aa, Artificial Sequence
  Description of Artificial Sequence Synthetic polypeptide
QIQLVQSGPELKKPGETVKISCKASGYTFTSYGVTWVKQAPGKGLKWMGWINTYSGVPTYADDFKGRFAFSLETSASTAYLQINNLKNEDTATYFCARSHSNYDFWGQGTTLTVSS

SEQ ID NO: 33 - protein, 107 aa, Artificial Sequence
  Description of Artificial Sequence Synthetic polypeptide
DIQMTQSPSSLSASLGERVSLTCRASQDIGNSLNWLQQKPDGTIKRLIYVTSRLDSGVPKRFSGSRSGSDYFFTISSLESEDFADYYCLQYASSPFTFGSGTKLEIK

SEQ ID NO: 34 - protein, 107 aa, Artificial Sequence
  Description of Artificial Sequence Synthetic polypeptide
DILLTQSPAILSVSPGERVSFSCRASQSIGTSLHWYQQRTNGSPRLLIKYASESISGIPSRFSGSGSGTDFTLSINSVESEDIADYYCQQSNSWPYTFGGGTKLEIK

SEQ ID NO: 35 - protein, 112 aa, Artificial Sequence
  Description of Artificial Sequence Synthetic polypeptide
DVLLTQTPLSLSVSLGDQASISCRSSQSFVHINGNTYLEWFLQKPGLSPKLLIYKVSNRFSGVPDRFSGSGSGTDFTLKISRVEAEDLGIYYCFQGSHVPLTFGAGTKLELK

SEQ ID NO: 36 - protein, 106 aa, Artificial Sequence
  Description of Artificial Sequence Synthetic polypeptide
EIVLTQSPAITAASLGQKVTITCSASSSVSDMHWYQQKSGTSPKPWIYEISKLASGVPTRFSGSGSGTSYSLTISSMEAEDAAIYYCQHWSYPLMTFGGGTKLEIK

SEQ ID NO: 37 - protein, 112 aa, Artificial Sequence
  Description of Artificial Sequence Synthetic polypeptide
DVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLEIK

SEQ ID NO: 38 - protein, 112 aa, Artificial Sequence
  Description of Artificial Sequence Synthetic polypeptide
DIVMTQAAFSNPVTLGTSASISCRSSKSLLHSNGFTYLHWYLQKPGQSPQLLIYQMSSLASGVPDRFSSSGSGTDFTLRISRVEAEDVGVYYCAQNLELPRTFGGGTKLEIK

SEQ ID NO: 39 - protein, 112 aa, Artificial Sequence
  Description of Artificial Sequence Synthetic polypeptide
DVLMTQSPLSLPVSLGDQASISCRSSQSIVDSDGSTYLEWYLQKPGQSPKLLIYKVSNRFSGVPDRFSGSGSGTDFTLKISGVEAEDLGVYYCFQGSHVPYTFGGGTKLEIK

SEQ ID NO: 40 - protein, 111 aa, Artificial Sequence
  Description of Artificial Sequence Synthetic polypeptide
DIVLTQSPASLAVSLGQRATISCRASESVDNSGISFMHWYQQKPGQSPKLLIYRASNLESGIPARFSGSGSRTDFTLTINPVETDDVATYYCQQSYENPWTFGGGTKLEIK

SEQ ID NO: 41 - protein, 111 aa, Artificial Sequence
  Description of Artificial Sequence Synthetic polypeptide
DIVLTQSPASLAVSLGQRATISCRASESVDNYGISFMHWFQQKPGQPPKLLIFRASSLESGIPARFSGSGSRTDFTLTINPVETDDVATYYCQQSYKDPWTFGGATKLEIK

SEQ ID NO: 42 - protein, 106 aa, Artificial Sequence
  Description of Artificial Sequence Synthetic polypeptide
EIVLTQSPAITAAFLGQKVTITCSASSSVSYIHWYQQKSDTSPKPWIYEISKLASGVPARFSGSGSGTSYSLTISSMEAEDAAIYYCQQWNYPLITFGGGTKLEIK

SEQ ID NO: 43 - protein, 111 aa, Artificial Sequence
  Description of Artificial Sequence Synthetic polypeptide
DIVLTQSPTSLAVSLGQRATISCRASESVDNYGISFMHWYQQKPGQPPKLLIYRASSLESGIPARFSGSGSRTDFTLTINPVESDDVATYYCQQSYKDPWTFGGATKLEIK

SEQ ID NO: 44 - protein, 107 aa, Artificial Sequence
  Description of Artificial Sequence Synthetic polypeptide
DIVMTQSQKFMSTTVGDRVSITCQASQNVGTDVGWYQQKPGQSPKLLIYSASNRYTGVPDRFTGSGSGTDFTLTISNMQSEDLADYFCQQYSSYPRTFGGGTKLEIK

SEQ ID NO: 45 - protein, 5 aa, Artificial Sequence
  Description of Artificial Sequence Synthetic peptide
SYDIN

SEQ ID NO: 46 - protein, 20 aa, Artificial Sequence
  Description of Artificial Sequence Synthetic peptide
WIGWIYPRDGSTKYNERFKG

SEQ ID NO: 47 - protein, 14 aa, Artificial Sequence
  Description of Artificial Sequence Synthetic peptide
DPYYYGTIYGYFDV

SEQ ID NO: 48 - protein, 5 aa, Artificial Sequence
  Description of Artificial Sequence Synthetic peptide
TYAMH

SEQ ID NO: 49 - protein, 19 aa, Artificial Sequence
  Description of Artificial Sequence Synthetic peptide
RIRSKTYNYATFYADSVKD

SEQ ID NO: 50 - protein, 8 aa, Artificial Sequence
  Description of Artificial Sequence Synthetic peptide
GSSYAMDY

SEQ ID NO: 51 - protein, 5 aa, Artificial Sequence
  Description of Artificial Sequence Synthetic peptide
SYWMH

SEQ ID NO: 52 - protein, 17 aa, Artificial Sequence
  Description of Artificial Sequence Synthetic peptide
VIDPSNSYSNYNQKFKG

SEQ ID NO: 53 - protein, 11 aa, Artificial Sequence
  Description of Artificial Sequence Synthetic peptide
PYNHGRDAMDY

SEQ ID NO: 54 - protein, 5 aa, Artificial Sequence
  Description of Artificial Sequence Synthetic peptide
SYGIS

SEQ ID NO: 55 - protein, 17 aa, Artificial Sequence
  Description of Artificial Sequence Synthetic peptide
EIYPRSDNTYYNEKFKG

SEQ ID NO: 56 - protein, 8 aa, Artificial Sequence
  Description of Artificial Sequence Synthetic peptide
RAGMWFAY

SEQ ID NO: 57 - protein, 6 aa, Artificial Sequence
  Description of Artificial Sequence Synthetic peptide
SDFAWN

SEQ ID NO: 58 - protein, 16 aa, Artificial Sequence
  Description of Artificial Sequence Synthetic peptide
YITYSGTTSYNPSLKS

SEQ ID NO: 59 - protein, 11 aa, Artificial Sequence
  Description of Artificial Sequence Synthetic peptide
SFHFFAYGFAY

SEQ ID NO: 60 - protein, 5 aa, Artificial Sequence
  Description of Artificial Sequence Synthetic peptide
SYGIS

SEQ ID NO: 61 - protein, 17 aa, Artificial Sequence
  Description of Artificial Sequence Synthetic peptide
EIYPTRDYTYYNENFKG

SEQ ID NO: 62 - protein, 7 aa, Artificial Sequence
  Description of Artificial Sequence Synthetic peptide
DGPPFAY

SEQ ID NO: 63 - protein, 5 aa, Artificial Sequence
  Description of Artificial Sequence Synthetic peptide
DYYMS

SEQ ID NO: 64 - protein, 17 aa, Artificial Sequence
  Description of Artificial Sequence Synthetic peptide
EINPDNGDTSHNQKFKG

SEQ ID NO: 65 - protein, 4 aa, Artificial Sequence
  Description of Artificial Sequence Synthetic peptide
YFSY

SEQ ID NO: 66 - protein, 5 aa, Artificial Sequence
  Description of Artificial Sequence Synthetic peptide
NTYIH

SEQ ID NO: 67 - protein, 17 aa, Artificial Sequence
  Description of Artificial Sequence Synthetic peptide
RIGPANGNTKYAPKFQG

SEQ ID NO: 68 - protein, 6 aa, Artificial Sequence
  Description of Artificial Sequence Synthetic peptide
AYYRIY

SEQ ID NO: 69 - protein, 5 aa, Artificial Sequence
  Description of Artificial Sequence Synthetic peptide
NTYMH

SEQ ID NO: 70 - protein, 17 aa, Artificial Sequence
  Description of Artificial Sequence Synthetic peptide
RIAPANGNTKYAPKFQD

SEQ ID NO: 71 - protein, 6 aa, Artificial Sequence
  Description of Artificial Sequence Synthetic peptide
IYQLPY

SEQ ID NO: 72 - protein, 5 aa, Artificial Sequence
  Description of Artificial Sequence Synthetic peptide
DYYIT

SEQ ID NO: 73 - protein, 17 aa, Artificial Sequence
  Description of Artificial Sequence Synthetic peptide
WIFPGRGISFYNENFKD

SEQ ID NO: 74 - protein, 8 aa, Artificial Sequence
  Description of Artificial Sequence Synthetic peptide
WGGTWFAY

SEQ ID NO: 75 - protein, 5 aa, Artificial Sequence
  Description of Artificial Sequence Synthetic peptide
NTYMH

SEQ ID NO: 76 - protein, 17 aa, Artificial Sequence
  Description of Artificial Sequence Synthetic peptide
RIAPANGNTKYDPKFQD

SEQ ID NO: 77 - protein, 6 aa, Artificial Sequence
  Description of Artificial Sequence Synthetic peptide
IYQLPY

SEQ ID NO: 78 - protein, 5 aa, Artificial Sequence
  Description of Artificial Sequence Synthetic peptide
SYGVT

SEQ ID NO: 79 - protein, 17 aa, Artificial Sequence
  Description of Artificial Sequence Synthetic peptide
WINTYSGVPTYADDFKG

SEQ ID NO: 80 - protein, 7 aa, Artificial Sequence
  Description of Artificial Sequence Synthetic peptide
SHSNYDF

SEQ ID NO: 81 - protein, 11 aa, Artificial Sequence
  Description of Artificial Sequence Synthetic peptide
RASQDIGNSLN

SEQ ID NO: 82 - protein, 7 aa, Artificial Sequence
  Description of Artificial Sequence Synthetic peptide
VTSRLDS

SEQ ID NO: 83 - protein, 9 aa, Artificial Sequence
  Description of Artificial Sequence Synthetic peptide
LQYASSPFT

SEQ ID NO: 84 - protein, 11 aa, Artificial Sequence
  Description of Artificial Sequence Synthetic peptide
RASQSIGTSLH

SEQ ID NO: 85 - protein, 7 aa, Artificial Sequence
  Description of Artificial Sequence Synthetic peptide
YASESIS

SEQ ID NO: 86 - protein, 9 aa, Artificial Sequence
  Description of Artificial Sequence Synthetic peptide
QQSNSWPYT

SEQ ID NO: 87 - protein, 16 aa, Artificial Sequence
  Description of Artificial Sequence Synthetic peptide
RSSQSFVHINGNTYLE

SEQ ID NO: 88 - protein, 7 aa, Artificial Sequence
  Description of Artificial Sequence Synthetic peptide
KVSNRFS

SEQ ID NO: 89 - protein, 5 aa, Artificial Sequence
  Description of Artificial Sequence Synthetic peptide
FQGSH

SEQ ID NO: 90 - protein, 10 aa, Artificial Sequence
  Description of Artificial Sequence Synthetic peptide
SASSSVSDMH

SEQ ID NO: 91 - protein, 7 aa, Artificial Sequence
  Description of Artificial Sequence Synthetic peptide
EISKLAS

SEQ ID NO: 92 - protein, 9 aa, Artificial Sequence
  Description of Artificial Sequence Synthetic peptide
QHWSYPLMT

SEQ ID NO: 93 - protein, 16 aa, Artificial Sequence
  Description of Artificial Sequence Synthetic peptide
KSSQSLLDSDGKTYLN

SEQ ID NO: 94 - protein, 7 aa, Artificial Sequence
  Description of Artificial Sequence Synthetic peptide
LVSKLDS

SEQ ID NO: 95 - protein, 5 aa, Artificial Sequence
  Description of Artificial Sequence Synthetic peptide
WQGTH

SEQ ID NO: 96 - protein, 16 aa, Artificial Sequence
  Description of Artificial Sequence Synthetic peptide
RSSKSLLHSNGFTYLH

SEQ ID NO: 97 - protein, 7 aa, Artificial Sequence
  Description of Artificial Sequence Synthetic peptide
QMSSLAS

SEQ ID NO: 98 - protein, 9 aa, Artificial Sequence
  Description of Artificial Sequence Synthetic peptide
AQNLELPRT

SEQ ID NO: 99 - protein, 16 aa, Artificial Sequence
  Description of Artificial Sequence Synthetic peptide
RSSQSIVDSDGSTYLE

SEQ ID NO: 100 - protein, 7 aa, Artificial Sequence
  Description of Artificial Sequence Synthetic peptide
KVSNRFS

SEQ ID NO: 101 - protein, 5 aa, Artificial Sequence
  Description of Artificial Sequence Synthetic peptide
FQGSH

SEQ ID NO: 102 - protein, 15 aa, Artificial Sequence
  Description of Artificial Sequence Synthetic peptide
RASESVDNSGISFMH

SEQ ID NO: 103 - protein, 7 aa, Artificial Sequence
  Description of Artificial Sequence Synthetic peptide
RASNLES

SEQ ID NO: 104 - protein, 9 aa, Artificial Sequence
  Description of Artificial Sequence Synthetic peptide
QQSYENPWT

SEQ ID NO: 105 - protein, 15 aa, Artificial Sequence
  Description of Artificial Sequence Synthetic peptide
RASESVDNYGISFMH

SEQ ID NO: 106 - protein, 7 aa, Artificial Sequence
  Description of Artificial Sequence Synthetic peptide
RASSLES

SEQ ID NO: 107 - protein, 6 aa, Artificial Sequence
  Description of Artificial Sequence Synthetic peptide
QQSYKD

SEQ ID NO: 108 - protein, 10 aa, Artificial Sequence
  Description of Artificial Sequence Synthetic peptide
SASSSVSYIH

SEQ ID NO: 109 - protein, 7 aa, Artificial Sequence
  Description of Artificial Sequence Synthetic peptide
EISKLAS

SEQ ID NO: 110 - protein, 9 aa, Artificial Sequence
  Description of Artificial Sequence Synthetic peptide
QQWNYPLIT

SEQ ID NO: 111 - protein, 15 aa, Artificial Sequence
  Description of Artificial Sequence Synthetic peptide
RASESVDNYGISFMH

SEQ ID NO: 112 - protein, 7 aa, Artificial Sequence
  Description of Artificial Sequence Synthetic peptide
RASSLES

SEQ ID NO: 113 - protein, 6 aa, Artificial Sequence
  Description of Artificial Sequence Synthetic peptide
QQSYKD

SEQ ID NO: 114 - protein, 11 aa, Artificial Sequence
  Description of Artificial Sequence Synthetic peptide
QASQNVGTDVG

SEQ ID NO: 115 - protein, 7 aa, Artificial Sequence
  Description of Artificial Sequence Synthetic peptide
SASNRYT

SEQ ID NO: 116 - protein, 9 aa, Artificial Sequence
  Description of Artificial Sequence Synthetic peptide
QQYSSYPRT

SEQ ID NO: 117 - protein, 464 aa, Artificial Sequence
  Description of Artificial Sequence Synthetic polypeptide
QVQLQQSGPELVKPGTSVKLSCRASGYTFTSYDINWVKQRPGQGLEWIGWIYPRDGSTKYNERFKGKATLTVDTSSSTAYMEFHTLTSEDSAVYFCARDPYYYGTIYGYFDVWGTGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSDYKDDDDK

SEQ ID NO: 118 - protein, 460 aa, Artificial Sequence
  Description of Artificial Sequence Synthetic polypeptide
EVQLVESGGGLVQPKGSLKLSCAASGFTFNTYAMHWVRQAPGKGLEWVARIRSKTYNYATFYADSVKDRFTISRDDSQSMLYLQMNNLKTEDTAMFYCVTGSSYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSDYKDDDDK

SEQ ID NO: 119 - protein, 458 aa, Artificial Sequence
  Description of Artificial Sequence Synthetic polypeptide
QVQLQQSGAELARPGASVQLSCKASGYTFTSYGISWLKQRAGQGLEWIGEIYPRSDNTYYNEKFKGKATLTADKSSSTAYMELRSLTSEDSAVYFCARRAGMWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSDYKDDDDK

SEQ ID NO: 120 - protein, 491 aa, Artificial Sequence
  Description of Artificial Sequence Synthetic polypeptide
EVQLLESGGGLVQPGGSLRLSCAASGFTFSNFAMSWVRQAPGKGLEWVSTISASGGRTFYADSVKGRFTISRDNSKNTLYLQMNSLRAEDTAVYYCAKEGSFGEGVDPWGQGTLVTVSSASTKGPRGSTSGGGSGGGSGGGGSSSYELTQPPSVSVSPGQTARITCSGDALPKKYAYWYQQKSGQAPVLVIYEDNKRPSGIPERFSGSSSGTMATLTISGAQVEDEADYYCYSTDISGNHWVFGGGTKLTVLASKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGSHHHHHHHH

SEQ ID NO: 121 - protein, 5 aa, Artificial Sequence
  Description of Artificial Sequence Synthetic peptide
NYGVH

SEQ ID NO: 122 - protein, 16 aa, Artificial Sequence
  Description of Artificial Sequence Synthetic peptide
VIWSGGNTDYNTPFTS

SEQ ID NO: 123 - protein, 11 aa, Artificial Sequence
  Description of Artificial Sequence Synthetic peptide
ALTYYDYEFAY

SEQ ID NO: 124 - protein, 11 aa, Artificial Sequence
  Description of Artificial Sequence Synthetic peptide
RASQSIGTNIH

SEQ ID NO: 125 - protein, 7 aa, Artificial Sequence
  Description of Artificial Sequence Synthetic peptide
YASESIS

SEQ ID NO: 126 - protein, 9 aa, Artificial Sequence
  Description of Artificial Sequence Synthetic peptide
QQNNNWPTT

SEQ ID NO: 127 - protein, 119 aa, Artificial Sequence
  Description of Artificial Sequence Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSA

SEQ ID NO: 128 - protein, 107 aa, Artificial Sequence
  Description of Artificial Sequence Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELK

SEQ ID NO: 129 - protein, 6 aa, Artificial Sequence
  Description of Artificial Sequence Synthetic 6xHis tag
HHHHHH

SEQ ID NO: 130 - protein, 449 aa, Artificial Sequence
  Description of Artificial Sequence Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - DNA, 1347 bp, Artificial Sequence
  Description of Artificial Sequence Synthetic polynucleotide
caggtgcagctgaagcagagcggccccggcctggtgcagccatcacagagcctgagcatcacatgcacagtgagcggattcagcctgacaaactacggcgtgcactgggtgagacagagccccggcaaaggactggagtggctgggagtgatttggtccggcggcaacaccgactacaacacccccttcacaagcaggctgagcatcaacaaggacaatagcaagagccaggtgttcttcaagatgaatagcctgcagtccaacgataccgccatctactactgcgccagagccctgacctactacgactacgaattcgcctactggggccagggaaccctggtgaccgtgagcgccgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctctggtgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcaag

SEQ ID NO: 132 - protein, 214 aa, Artificial Sequence
  Description of Artificial Sequence Synthetic polypeptide
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - DNA, 642 bp, Artificial Sequence
  Description of Artificial Sequence Synthetic polynucleotide
gacatattgttgacgcaaagcccggttatacttagtgttagtccgggtgaaagagtatctttttcttgtcgcgcttctcagtctatcggaacaaacattcattggtatcaacaacggacaaacggaagccctagacttctcataaaatacgcctcagagtccatcagtggcataccttctcgattttcaggctctgggtctggtactgacttcacattgtctatcaattccgtcgaatctgaggacatagcggactattattgccagcagaataataactggccaactacttttggtgctggaactaagttggagcttaagcgtacggtggcagctccatcagtttttatcttcccaccaagcgacgagcaattgaagtccggcactgcctctgtggtgtgccttctgaacaacttctatccaagggaggccaaggtccagtggaaggtcgataatgcgctgcagagcgggaacagccaagagtcagtgaccgagcaggactcaaaagatagcacatactctctgagttccaccctgaccctgtcaaaggctgactacgaaaagcataaggtatacgcatgcgaagtgacccatcagggtctctcatctcccgtaaccaaatcttttaatagaggagaatgc

SEQ ID NO: 134 - protein, 488 aa, Artificial Sequence
  Description of Artificial Sequence Synthetic polypeptide
EVQLLESGGGLVQPGGSLRLSCAASGFTFSNFAMSWVRQAPGKGLEWVSTISASGGRTFYADSVKGRFTISRDNSKNTLYLQMNSLRAEDTAVYYCAKEGSFGEGVDPWGQGTLVTVSSASTKGPRGSTSGGGSGGGSGGGGSSSYELTQPPSVSVSPGQTARITCSGDALPKKYAYWYQQKSGQAPVLVIYEDNKRPSGIPERFSGSSSGTMATLTISGAQVEDEADYYCYSTDISGNHWVFGGGTKLTVLGGSGGSGGASKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 135 - DNA, 1464 bp, Artificial Sequence
  Description of Artificial Sequence Synthetic polynucleotide
gaggtacaacttctggagtcaggcggggggctagtccaacctgggggctcgctacggttatcttgcgccgcgtctggttttaccttcagtaactttgccatgtcatgggttagacaagcacctggcaagggcctcgagtgggtatcgactatttccgccagcggaggtcgaacattttacgcagactcggtgaaaggtcgcttcacaatttctagggataattctaaaaacaccctgtatcttcaaatgaacagcctgcgtgccgaggacaccgcagtttactactgcgcgaaagaaggttcatttggtgaaggcgtcgatccatggggtcaggggaccttagttacagtttcgtccgcgagcacgaagggtcctaggggttcgaccagcggaggaggtagtgggggaggctcaggtgggggagggtccagcagctacgagctaacccaaccaccatcagtatccgtgtctccgggccaaacggcgagaatcacttgcagcggagacgcactaccgaaaaagtatgcttattggtaccaacaaaaaagcggacaggcgccggttctcgtcatatatgaagacaacaagcgcccttctggtattccggagaggttctccgggtcgtcatcaggaacaatggcgacgcttaccatatcgggagcacaagtagaagatgaagcggactactactgctattctactgacatctctggaaaccactgggtcttcggaggtggtaccaagcttaccgtccttggtggcagtggtggctctgggggcgctagcaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcagctgcgcggttaaaggattttatccaagcgatattgctgtggagtgggagtccaacggccagcctgagaacaattataaaaccaccccccctgttcttgacagtgacggtagtttcttcctggtttccaaactgaccgtcgataagagcagatggcaacagggaaatgtgttcagctgctccgtgatgcatgaggcgctccataatcattacacacaaaaaagtttgtccctgagcccaggcaag

SEQ ID NO: 136 - protein, 460 aa, Artificial Sequence
  Description of Artificial Sequence Synthetic polypeptide
EVQLLESGGGLVQPGGSLRLSCAASGFTFSNFAMSWVRQAPGKGLEWVSTISASGGRTFYADSVKGRFTISRDNSKNTLYLQMNSLRAEDTAVYYCAKEGSFGEGVDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGGGGSDYKDDDDK

SEQ ID NO: 137 - DNA, 1380 bp, Artificial Sequence
  Description of Artificial Sequence Synthetic polynucleotide
gaggtgcagctgctggagagcggggggggactggtgcagccaggaggaagcctgagactgagctgtgccgccagcgggtttacatttagcaactttgccatgagctgggtgagacaggcccccggaaaaggactggagtgggtgagcaccattagcgccagcggaggaagaacattttacgctgattccgtgaaggggagattcacaatctccagagacaacagcaagaacaccctgtacctgcagatgaacagcctgagagccgaggacaccgccgtgtattactgcgccaaggagggatcttttggcgagggagtggacccctggggccagggaaccctggtgaccgtgagcagcgctagcaccaaaggtcccagcgtgttcccactcgccccgagttcaaaatcaacttctggaggcaccgccgccctgggttgcctggtaaaggactacttcccagagcccgtgaccgtgagctggaactccggggcactgacatctggcgttcatactttcccggccgtgctccagtcttcaggtctgtatagtctctcctctgtggtcactgtcccatctagctctctgggcacccaaacctacatatgcaacgttaatcacaagccgagcaatactaaagttgacaaaaaggtggaacccaagtcttgtgacaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctctggtgcttggttaaaggattttatccaagcgatattgctgtggagtgggagtccaacggccagcctgagaacaattataaaaccaccccccctgttcttgacagtgacggtagtttcttcctgtattccaaactgaccgtcgataagagcagatggcaacagggaaatgtgttcagctgctccgtgatgcatgaggcgctccataatcattacacacaaaaaagtttgtccctgagcccaggcggcggaggcagcgactacaaggacgacgatgataag

SEQ ID NO: 138 - DNA, 645 bp, Artificial Sequence
  Description of Artificial Sequence Synthetic polynucleotide
tcttacgagctgacccagcctccttctgtgtctgtgtctcccggtcagaccgctagaatcacctgctctggcgacgctctgcctaagaagtacgcctactggtaccagcagaagtccggccaggctcctgtgctggtgatttacgaggacaataagagaccctccggcatccccgagagattctctggatcctcctccggaaccatggccacactgactatctccggcgctcaggtggaggatgaagctgattattactgctactccaccgacatctccggcaaccactgggtgttcggaggtggaacaaagctgaccgtgctgggacagcctaaggctgctccttctgtgactctgtttcctcccagctccgaggagctgcaggctaacaaagctactctggtctgcctgatcagtgatttctaccccggcgccgtcaccgtggcttggaaggctgattcttcccccgtgaaggccggtgtggaaactacaacaccctccaaacagtccaacaacaaatacgccgcctcctcctacctgtccctgacacctgaacagtggaagtctcacagatcctactcctgccaggtcacccacgaaggatccacagtcgagaagaccgtcgcccctacagagtgttcttga

SEQ ID NO: 139 - protein, 498 aa, Artificial Sequence
  Description of Artificial Sequence Synthetic polypeptide
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPRGSTSGGGSGGGSGGGGSSDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGSGGSGGASKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGGGGSHHHHHHHH

SEQ ID NO: 140 - DNA, 1494 bp, Artificial Sequence
  Description of Artificial Sequence Synthetic polynucleotide
caggtgcagctgaagcagagcggccccggcctggtgcagccatcacagagcctgagcatcacatgcacagtgagcggattcagcctgacaaactacggcgtgcactgggtgagacagagccccggcaaaggactggagtggctgggagtgatttggtccggcggcaacaccgactacaacacccccttcacaagcaggctgagcatcaacaaggacaatagcaagagccaggtgttcttcaagatgaatagcctgcagtccaacgataccgccatctactactgcgccagagccctgacctactacgactacgaattcgcctactggggccagggaaccctggtgaccgtgagcgccgcttctaccaaaggaccccggggctctacctccggcggcggaagcggaggaggatctggaggaggaggaagttctgacatattgttgacgcaaagcccggttatacttagtgttagtccgggtgaaagagtatctttttcttgtcgcgcttctcagtctatcggaacaaacattcattggtatcaacaacggacaaacggaagccctagacttctcataaaatacgcctcagagtccatcagtggcataccttctcgattttcaggctctgggtctggtactgacttcacattgtctatcaattccgtcgaatctgaggacatagcggactattattgccagcagaataataactggccaactacttttggtgctggaactaagttggagcttaagggtggcagtggtggctctgggggcgctagcaagacccacacgtgtcccccctgcccggctcctgagctgcttggcggccccagcgtctttctctttcccccaaagccaaaagataccttgatgatcagcagaactcccgaggtgacatgcgtcgtcgtggacgtaagccatgaagatcccgaggttaagttcaactggtatgtcgatggcgtggaagtccataatgctaagactaaacctcgcgaagagcagtacaattcaacttaccgggtcgtttccgttctgaccgtgctgcatcaggactggctgaatggtaaagagtacaagtgcaaagtgtctaacaaggcactccccgccccaattgagaagactatctccaaagctaaagggcaaccaagagagccccaggtctacaccctgcccccctcaagggatgagcttactaagaaccaggttagtctcagctgcgcggttaaaggattttatccaagcgatattgctgtggagtgggagtccaacggccagcctgagaacaattataaaaccaccccccctgttcttgacagtgacggtagtttcttcctggtttccaaactgaccgtcgataagagcagatggcaacagggaaatgtgttcagctgctccgtgatgcatgaggcgctccataatcattacacacaaaaaagtttgtccctgagcccaggcggcggaggcagccaccaccaccaccaccaccatcat

SEQ ID NO: 141 - protein, 119 aa, Artificial Sequence
  Description of Artificial Sequence Synthetic polypeptide
EVQLLESGGGLVQPGGSLRLSCAASGFTFSNFAMSWVRQAPGKGLEWVSTISASGGRTFYADSVKGRITISRDNSKNTLFLQMNGLRAEDTAVYYCAAEGSFGEGVDPWGQGTLVTVSS

SEQ ID NO: 142 - protein, 119 aa, Artificial Sequence
  Description of Artificial Sequence Synthetic polypeptide
EVQLLESGGGLVQPGGSLRLSCAASGFTFSNFAMSWVRQAPGKGLEWVSTISASGGRTFYADSVKGRITISRDNSKNTLFLQMNGLRAEDTAVYYCAKAGSFGEGVDPWGQGTLVTVSS

SEQ ID NO: 143 - protein, 119 aa, Artificial Sequence
  Description of Artificial Sequence Synthetic polypeptide
EVQLLESGGGLVQPGGSLRLSCAASGFTFSNFAMSWVRQAPGKGLEWVSTISASGGRTFYADSVKGRITISRDNSKNTLFLQMNGLRAEDTAVYYCAKEASFGEGVDPWGQGTLVTVSS

SEQ ID NO: 144 - protein, 119 aa, Artificial Sequence
  Description of Artificial Sequence Synthetic polypeptide
EVQLLESGGGLVQPGGSLRLSCAASGFTFSNFAMSWVRQAPGKGLEWVSTISASGGRTFYADSVKGRITISRDNSKNTLFLQMNGLRAEDTAVYYCAKEGAFGEGVDPWGQGTLVTVSS

SEQ ID NO: 145 - protein, 119 aa, Artificial Sequence
  Description of Artificial Sequence Synthetic polypeptide
EVQLLESGGGLVQPGGSLRLSCAASGFTFSNFAMSWVRQAPGKGLEWVSTISASGGRTFYADSVKGRITISRDNSKNTLFLQMNGLRAEDTAVYYCAKEGSAGEGVDPWGQGTLVTVSS

SEQ ID NO: 146 - protein, 119 aa, Artificial Sequence
  Description of Artificial Sequence Synthetic polypeptide
EVQLLESGGGLVQPGGSLRLSCAASGFTFSNFAMSWVRQAPGKGLEWVSTISASGGRTFYADSVKGRITISRDNSKNTLFLQMNGLRAEDTAVYYCAKEGSFAEGVDPWGQGTLVTVSS

SEQ ID NO: 147 - protein, 119 aa, Artificial Sequence
  Description of Artificial Sequence Synthetic polypeptide
EVQLLESGGGLVQPGGSLRLSCAASGFTFSNFAMSWVRQAPGKGLEWVSTISASGGRTFYADSVKGRITISRDNSKNTLFLQMNGLRAEDTAVYYCAKEGSFGAGVDPWGQGTLVTVSS

SEQ ID NO: 148 - protein, 119 aa, Artificial Sequence
  Description of Artificial Sequence Synthetic polypeptide
EVQLLESGGGLVQPGGSLRLSCAASGFTFSNFAMSWVRQAPGKGLEWVSTISASGGRTFYADSVKGRITISRDNSKNTLFLQMNGLRAEDTAVYYCAKEGSFGEAVDPWGQGTLVTVSS

SEQ ID NO: 149 - protein, 119 aa, Artificial Sequence
  Description of Artificial Sequence Synthetic polypeptide
EVQLLESGGGLVQPGGSLRLSCAASGFTFSNFAMSWVRQAPGKGLEWVSTISASGGRTFYADSVKGRITISRDNSKNTLFLQMNGLRAEDTAVYYCAKEGSFGEGADPWGQGTLVTVSS

SEQ ID NO: 150 - protein, 119 aa, Artificial Sequence
  Description of Artificial Sequence Synthetic polypeptide
EVQLLESGGGLVQPGGSLRLSCAASGFTFSNFAMSWVRQAPGKGLEWVSTISASGGRTFYADSVKGRITISRDNSKNTLFLQMNGLRAEDTAVYYCAKEGSFGEGVAPWGQGTLVTVSS

SEQ ID NO: 151 - protein, 119 aa, Artificial Sequence
  Description of Artificial Sequence Synthetic polypeptide
EVQLLESGGGLVQPGGSLRLSCAASGFTFSNFAMSWVRQAPGKGLEWVSTISASGGRTFYADSVKGRITISRDNSKNTLFLQMNGLRAEDTAVYYCAKEGSFGEGVDAWGQGTLVTVSS

SEQ ID NO: 152 - protein, 108 aa, Artificial Sequence
  Description of Artificial Sequence Synthetic polypeptide
SYEMTQPPSVSVSPGQTARITCSGDALPKKYAYWYQQKSGQAPVLVIYEDNKRPSGIPERFSGSSSGTMATLTISGAQVEDEADYYCYSTDISGNHWVFGGGTELTVL

SEQ ID NO: 153 - protein, 108 aa, Artificial Sequence
  Description of Artificial Sequence Synthetic polypeptide
SYEMTQPPSVSVSPGQTARITCSGDALPKKYAYWYQQKSGQAPVLVIYEDNKRPSGIPERFSGSSSGTMATLTISGAQVEDEADYYCASTDISGNHWVFGGGTELTVL

SEQ ID NO: 154 - protein, 108 aa, Artificial Sequence
  Description of Artificial Sequence Synthetic polypeptide
SYEMTQPPSVSVSPGQTARITCSGDALPKKYAYWYQQKSGQAPVLVIYEDNKRPSGIPERFSGSSSGTMATLTISGAQVEDEADYYCYATDISGNHWVFGGGTELTVL

SEQ ID NO: 155 - protein, 108 aa, Artificial Sequence
  Description of Artificial Sequence Synthetic polypeptide
SYEMTQPPSVSVSPGQTARITCSGDALPKKYAYWYQQKSGQAPVLVIYEDNKRPSGIPERFSGSSSGTMATLTISGAQVEDEADYYCYSADISGNHWVFGGGTELTVL

SEQ ID NO: 156 - protein, 108 aa, Artificial Sequence
  Description of Artificial Sequence Synthetic polypeptide
SYEMTQPPSVSVSPGQTARITCSGDALPKKYAYWYQQKSGQAPVLVIYEDNKRPSGIPERFSGSSSGTMATLTISGAQVEDEADYYCYSTAISGNHWVFGGGTELTVL

SEQ ID NO: 157 - protein, 108 aa, Artificial Sequence
  Description of Artificial Sequence Synthetic polypeptide
SYEMTQPPSVSVSPGQTARITCSGDALPKKYAYWYQQKSGQAPVLVIYEDNKRPSGIPERFSGSSSGTMATLTISGAQVEDEADYYCYSTDASGNHWVFGGGTELTVL

SEQ ID NO: 158 - protein, 108 aa, Artificial Sequence
  Description of Artificial Sequence Synthetic polypeptide
SYEMTQPPSVSVSPGQTARITCSGDALPKKYAYWYQQKSGQAPVLVIYEDNKRPSGIPERFSGSSSGTMATLTISGAQVEDEADYYCYSTDIAGNHWVFGGGTELTVL

SEQ ID NO: 159 - protein, 108 aa, Artificial Sequence
  Description of Artificial Sequence Synthetic polypeptide
SYEMTQPPSVSVSPGQTARITCSGDALPKKYAYWYQQKSGQAPVLVIYEDNKRPSGIPERFSGSSSGTMATLTISGAQVEDEADYYCYSTDISANHWVFGGGTELTVL

SEQ ID NO: 160 - protein, 108 aa, Artificial Sequence
  Description of Artificial Sequence Synthetic polypeptide
SYEMTQPPSVSVSPGQTARITCSGDALPKKYAYWYQQKSGQAPVLVIYEDNKRPSGIPERFSGSSSGTMATLTISGAQVEDEADYYCYSTDISGAHWVFGGGTELTVL

SEQ ID NO: 161 - protein, 108 aa, Artificial Sequence
  Description of Artificial Sequence Synthetic polypeptide
SYEMTQPPSVSVSPGQTARITCSGDALPKKYAYWYQQKSGQAPVLVIYEDNKRPSGIPERFSGSSSGTMATLTISGAQVEDEADYYCYSTDISGNAWVFGGGTELTVL

SEQ ID NO: 162 - protein, 108 aa, Artificial Sequence
  Description of Artificial Sequence Synthetic polypeptide
SYEMTQPPSVSVSPGQTARITCSGDALPKKYAYWYQQKSGQAPVLVIYEDNKRPSGIPERFSGSSSGTMATLTISGAQVEDEADYYCYSTDISGNHAVFGGGTELTVL

SEQ ID NO: 163 - protein, 108 aa, Artificial Sequence
  Description of Artificial Sequence Synthetic polypeptide
SYEMTQPPSVSVSPGQTARITCSGDALPKKYAYWYQQKSGQAPVLVIYEDNKRPSGIPERFSGSSSGTMATLTISGAQVEDEADYYCYSTDISGNHWAFGGGTELTVL

(19) United States
(12) Patent Application Publication
(10) Pub. No.: US 2023/0159663 A1
(43) Pub. Date: May 25, 2023

(54) BISPECIFIC ANTIBODIES FOR TREATING CD47-ASSOCIATED DISEASES

(71) Applicant: Virtuoso BINco, Inc., San Mateo, CA (US)

(72) Inventors: Xinhua WANG, San Mateo, CA (US); Xiaocheng CHEN, San Mateo, CA (US); Lei SHI, Shanghai (CN); Leonard POST, San Mateo, CA (US); Oi Kwan WONG, San Mateo, CA (US)

(21) Appl. No.: 17/996,837

(22) PCT Filed: Apr. 23, 2021
(86) PCT No.: PCT/CN2021/089397

§ 371 Date: Oct. 21, 2022

(30) Foreign Application Priority Data
Apr. 24, 2020 (CN) PCT/CN2020/086815

(51) Int. Cl. C07K 16/46; A61P 35/00

(52) U.S. Cl. CPC C07K 16/468; A61P 35/00; C07K 2317/52; C07K 2317/92; C07K 2317/622; C07K 2317/55; C07K 2317/565

(57) ABSTRACT

Disclosed herein are bispecific antibodies comprising a first targeting moiety that specifically binds to CD47 and a second targeting moiety that specifically binds to EpCAM or EGFR and, pharmaceutical compositions and methods comprising the bispecific antibodies.

### 1. CROSS-REFERENCE

This application claims the benefit of International Patent Application No. PCT/CN2020/086815 filed Apr. 24, 2020, which is entirely incorporated herein by reference for all purposes.

### 1.1. SEQUENCE LISTING

The instant application contains a Sequence Listing which has been submitted electronically in ASCII format and is hereby incorporated by reference in its entirety. Said ASCII copy, created on Apr. 21, 2021, is named 55429-708_602_SL.txt and is 157,978 bytes in size.

### 2. SUMMARY OF THE INVENTION

This disclosure provides bispecific proteins (e.g. bispecific antibodies) with a CD47-binding domain and an EpCAM binding domain. In some embodiments, various domains of the bispecific protein have sequences of human origin and may therefore induce a weaker immune response than analogous domains with non-human sequences. These bispecific proteins can bind to CD47 on the surface of a cell, and can block a CD47-SIRPα interaction and permitting phagocytosis of the CD47+ cells. In some embodiments, the CD47-binding domain preferentially binds to CD47 on malignant cells over that on RBCs or platelets.

EpCAM is a tumor-associated glycoprotein that is highly expressed on some cancer cells and is expressed at lower or zero levels on some normal cells. On some polarized epithelial cells, EpCAM is expressed on an apical surface which is inaccessible to circulating therapeutic proteins. Its expression pattern changes in cancer to an intense uniform membranous over-expression.

In some embodiments, a bispecific CD47×EpCAM antibody blocks don't-eat-me signaling and thereby increases phagocytosis of a CD47+/EpCAM+ cell. In some embodiments, a bispecific CD47×EpCAM antibody comprises an IgG1 Fc domain that mediates antibody-dependent cellular cytotoxicity (ADCC) of a CD47+/EpCAM+ cell.

Described herein, in certain embodiments, are compositions comprising a bispecific antibody, wherein the bispecific antibody comprises a CD47-binding domain and an EpCAM binding domain. In some embodiments, the bispecific antibody further comprises an Fc domain. In some embodiments, the bispecific antibody has a higher affinity for CD47 expressed on the surface of a tumor cell than for CD47 expressed on the surface of a red blood cell or a platelet. In some embodiments, the tumor cell expresses EpCAM. In some embodiments, a concentration of the bispecific antibody required for half-maximal binding to a human red blood cell is greater than 500 nM. In some embodiments, the bispecific antibody binds to human CD47 with a KD of less than 100 nM. In some embodiments, the bispecific antibody binds to human CD47 with a KD of between 1 nM and 100 nM, between 1 nM and 50 nM, or between 5 nM and 50 nM. In some embodiments, the bispecific antibody binds to EpCAM with a KD of less than 500 nM. In some embodiments, the bispecific antibody binds to EpCAM with a KD of less than 25 nM. In some embodiments, the bispecific antibody binds to EpCAM with a KD of between 0.2 nM and 500 nM, between 1 nM and 300 nM, between 5 nM and 200 nM, or between 10 nM and 150 nM. In some embodiments, the KD is determined by surface plasmon resonance. In some embodiments, the CD47-binding domain is a human or engineered human CD47-binding domain. In some embodiments, the CD47-binding domain comprises a heavy chain variable domain and a light chain variable domain. In some embodiment, the CD47-binding domain comprises an scFv. In some embodiments, the EpCAM-binding domain comprises a heavy chain variable domain and a light chain variable domain. In some embodiments, the EpCAM-binding domain comprises an scFv. In some embodiments, the Fc domain is a human Fc domain. In some embodiments, the isotype of the human Fc domain is IgG1 or IgG4. In some embodiments, the Fc domain is a heterodimeric Fc domain, wherein the heterodimeric Fc region comprises a knob chain and a hole chain, forming a knob-into-hole (KiH) structure. In some embodiments, the knob chain comprises the mutation T366W and the hole chain comprises the mutations T366S, L368A, and Y407V, wherein amino acid position numbering is according to the EU index of Kabat et al. In some embodiments, the bispecific antibody has an asymmetric three-chain knob-into-hole structure. In some embodiments, the CD47-binding domain is an scFv. In some embodiments, the EpCAM-binding domain is an scFv. In some embodiments, the CD47-binding domain comprises three heavy chain (HC) complementarity determining regions (CDRs): HC-CDR1, HC-CDR2, and HC-CDR3; and three light chain (LC) CDRs: LC-CDR1, the LC-CDR2, and the LC-CDR3; wherein HC-CDR1 comprises SEQ ID NO: 15, HC-CDR2 comprises SEQ ID NO: 16, HC-CDR3 comprises a sequence with at least 50% identity to SEQ ID NO: 17, LC-CDR1 comprises SEQ ID NO: 18, LC-CDR2 comprises SEQ ID NO: 19, and LC-CDR3 comprises a sequence with at least 50% identity to SEQ ID NO: 20. In some embodiments, the HC-CDR3 comprises amino acid substitutions at one or more of K94, E95, G96, S97, F98, G99, V100b, D101, and P102; and LC-CDR3 comprises amino acid substitutions at one or more of Y89, S90, T91, D92, 193, S94, G95, N95a, H95b, W96, and V97. In some embodiments, the heavy chain variable domain of the CD47-binding domain comprises a sequence with at least 90% identity to SEQ ID NO: 5, SEQ ID NO: 10, or SEQ ID NO: 13 and the light chain of the CD47-binding domain comprises a sequence with at least 90% identity to SEQ ID NO: 8, SEQ ID NO: 11, or SEQ ID NO: 14. In some embodiments, the EpCAM-binding domain comprises three heavy chain (HC) complementarity determining regions (CDRs), HC-CDR1, HC-CDR2, and HC-CDR3 and three light chain (LC) CDRs, LC-CDR1, the LC-CDR2, and the LC-CDR3; wherein the CDRs are defined by an ordered set of sequences listed as HC-CDR1, HC-CDR2, HC-CDR3, LC-CDR1, LC-CDR2, LC-CDR3; and wherein the CDRs are selected from the group consisting of sequences having at least 90% identity to: SEQ ID NO: 45, SEQ ID NO: 46, SEQ ID NO: 47, SEQ ID NO: 81, SEQ ID NO: 82, and SEQ ID NO: 83; SEQ ID NO: 48, SEQ ID NO: 49, SEQ ID NO: 50, SEQ ID NO: 84, SEQ ID NO: 85, and SEQ ID NO: 86; SEQ ID NO: 51, SEQ ID NO: 52, SEQ ID NO: 53, SEQ ID NO: 87, SEQ ID NO: 88, and SEQ ID NO: 89; SEQ ID NO: 54, SEQ ID NO: 55, SEQ ID NO: 56, SEQ ID NO: 90, SEQ ID NO: 91, and SEQ ID NO: 92; SEQ ID NO: 57, SEQ ID NO: 58, SEQ ID NO: 59, SEQ ID NO: 93, SEQ ID NO: 94, and SEQ ID NO: 95; SEQ ID NO: 60, SEQ ID NO: 61, SEQ ID NO: 62, SEQ ID NO: 96, SEQ ID NO: 97, and SEQ ID NO: 98; SEQ ID NO: 63, SEQ ID NO: 64, SEQ ID NO: 65, SEQ ID NO: 99, SEQ ID NO: 100, and SEQ ID NO: 101; SEQ ID NO: 66, SEQ ID NO: 67, SEQ ID NO: 68, SEQ ID NO: 102, SEQ ID NO: 103, and SEQ ID NO: 104; SEQ ID NO: 69, SEQ ID NO: 70, SEQ ID NO: 71, SEQ ID NO: 105, SEQ ID NO: 106, and SEQ ID NO: 107; SEQ ID NO: 72, SEQ ID NO: 72, SEQ ID NO: 74, SEQ ID NO: 108, SEQ ID NO: 109, and SEQ ID NO: 110; SEQ ID NO: 75, SEQ ID NO: 76, SEQ ID NO: 77, SEQ ID NO: 111, SEQ ID NO: 112, and SEQ ID NO: 113; and SEQ ID NO: 78, SEQ ID NO: 79, SEQ ID NO: 80, SEQ ID NO: 114, SEQ ID NO: 115, and SEQ ID NO: 116. In some embodiments, the EpCAM-binding domain comprises: a sequence at least 90% identical to SEQ ID NO: 21 and a sequence at least 90% identical to SEQ ID NO: 33; a sequence at least 90% identical to SEQ ID NO: 22 and a sequence at least 90% identical to SEQ ID NO: 34; or a sequence at least 90% identical to SEQ ID NO: 24 and a sequence at least 90% identical to SEQ ID NO: 36. In some embodiments, less than 1 nM or less than 0.1 nM of the bispecific antibody increases a percentage of A431 cells engulfed by macrophage by at least 4-fold compared to a nonspecific IgG1 antibody control. In some embodiments, a concentration of the antibody required to mediate antibody-dependent cellular phagocytosis of an EpCAM-positive, CD47-positive tumor cell by a macrophage is between 0.01 nM and −3 nM. In some embodiments, the EpCAM-positive, CD47-positive tumor cell is an OVISE cell or an A431 cell. In some embodiments, the EpCAM-positive, CD47-positive tumor cell is selected from a ductal pancreatic adenocarcinoma cell, an ovarian clear cell adenocarcinoma cell, a colon adenocarcinoma cell, a lung adenocarcinoma cell, an ovarian adenocarcinoma cell, a vulvar squamous cell carcinoma cell. In some embodiments, 100 nM of the bispecific antibody inhibits the binding of SIRPα to CD47 on the surface of a cell by at least 30%. In some embodiments, the cell is a CD47+ EpCAM+ tumor cell. In some embodiments, the cell expresses at least as many EpCAM proteins on its surface as a CFPAC-1 cell or an OVISE cell. In some embodiments, the cell expresses at least 100,000, at least 300,000, at least 600,000 or at least 1,500,000 EpCAM proteins on its surface. In some embodiments, 400 nM of the bispecific antibody does not induce hemolysis of red blood cells in a hemagglutination assay.

Some embodiments provide a complex comprising said bispecific antibody, according to any of the above embodiments, and a CD47+ EpCAM+ target cell. In some embodiments, the target cell expresses at least as many EpCAM proteins on its surface as an HCC-44 cell. In some embodiments, the target cell expresses at least 100,000, at least 300,000, at least 600,000 or at least 1,500,000 EpCAM proteins on its surface. In some embodiments, the CD47+ EpCAM+ target cell is a cancer cell. In some embodiments, the cancer cell is a ductal pancreatic adenocarcinoma cell, an ovarian clear cell adenocarcinoma cell, a colon adenocarcinoma cell, a lung adenocarcinoma cell, an ovarian adenocarcinoma cell, or a vulvar squamous cell carcinoma cell.

One embodiment provides a method of inducing phagocytosis of a CD47+ EpCAM+ target cell comprising administering said bispecific antibody according any of the above embodiments. In some embodiments, the bispecific antibody is administered at a concentration of less than 5 nM, less than 2 nM, less than 1 nM, less than 0.5 nM, less than 0.2 nM, less than 0.1 nM, or less than 0.05 nM.

Some embodiments provide a method of killing a CD47+ EpCAM+ target cell comprising administering said bispecific antibody according to any of the above embodiments, wherein the bispecific antibody inhibits the binding of SIRPα to CD47 on the surface of the CD47+ EpCAM+ target cell.

Some embodiments provide a method of killing a CD47+ EpCAM+ target cell comprising administering said bispecific antibody according to any of the above embodiments, wherein the bispecific antibody induces antibody-dependent cellular cytotoxicity that kills the CD47+ EpCAM+ target cell. In some embodiments, the antibody is administered at a concentration of between 0.01-1 nM, between 0.01-0.5 nM, between 0.01-0.25 nM, between 0.01-0.1 nM, between 0.01-0.05 nM, or less than 0.01 nM. In some embodiments, the CD47+ EpCAM+ target cell is selected from an A431 cell, a HCC-44 cell, a SKOV-3 cell, an OVISE cell, or a CFPAC-1 cell. In some embodiments, the CD47+ EpCAM+ target cell is selected from a ductal pancreatic adenocarcinoma cell, an ovarian clear cell adenocarcinoma cell, a colon adenocarcinoma cell, a lung adenocarcinoma cell, an ovarian adenocarcinoma cell, or a vulvar squamous cell carcinoma cell.

Some embodiments provide a pharmaceutical comprising said bispecific antibody according to any of the above embodiments. Another embodiment provides a method of treating an individual with cancer comprising administering said pharmaceutical composition. In some embodiments, the cancer is ductal pancreatic adenocarcinoma, ovarian clear cell adenocarcinoma, colon adenocarcinoma, lung adenocarcinoma cell, ovarian adenocarcinoma, or vulvar squamous cell carcinoma.

### 3. BRIEF DESCRIPTION OF THE FIGURES

FIG. 1. Illustration of the structure of a three-chain knob-into-hole EpCAM×CD47 bispecific antibody.

FIG. 2. Antibody Dependent Cellular Phagocytosis (ADCP) of CFPAC-1 pancreatic adenocarcinoma cells as determined by flow cytometry.

FIG. 3. Sequence alignment of the heavy chain ((FIG. 3A) and light chain (FIG. 3B) alanine scanning mutants of the αCD47 antibody VIR47. FIG. 3A discloses SEQ ID NOS 5 and 141-151, respectively, in order of appearance. FIG. 3B discloses SEQ ID NOS 152-163, respectively, in order of appearance.

FIGS. 4A-4B. Binding of bispecific antibody, Bi-1, to human CD47 (FIG. 4A) and human EpCAM (FIG. 4B) as measured by ELISA.

FIGS. 5A-5B. Inhibition of SIRPα binding to OVISE cells (FIG. 5A) or Raji cells (FIG. 5B) by bispecific or bivalent anti-CD47 antibodies.

FIG. 6. Antibody dependent cellular phagocytosis (ADCP) of A431 cells treated with the indicated bispecific, bivalent, and/or monovalent antibodies.

FIG. 7. Hemagglutination of human red blood cells by the indicated bispecific, bivalent antibodies.

FIGS. 8A-8B. Binding of bispecific, bivalent antibodies to red blood cells (FIG. 8A) and platelets (FIG. 8B)

FIGS. 9A-9B. Illustration of the structure of a three-chain knob-into-hole anti-EGFRxCD47 bispecific antibody

FIGS. 10A-10B. Binding of anti-CD47/EGFR bispecific antibodies on huEGFR (FIG. 10A) and human CD47 (FIG. 10B) by ELISA.

FIG. 11. Inhibition of SIRPα binding to A431 cells by anti-CD47/EGFR bispecific antibody as compared to various controls.

FIG. 12. Antibody Dependent Cellular Phagocytosis (ADCP) of A431 cells by anti-CD47/EGFR bispecific antibody as compared to various controls.

FIGS. 13A-13B. Binding of anti-CD47/EGFR bispecific antibody to human RBC cells (FIG. 13A) and human platelet cells (FIG. 13B) as compared to various controls.

FIG. 14. In vivo anti-tumor activity of anti-CD47/EGFR bispecific antibody in a subcutaneous gastric cancer model SNU-5 as compared to various controls.

### 4. DETAILED DESCRIPTION OF THE DISCLOSURE

Cluster of differentiation 47 (CD47), also known as integrin-associated protein (TAP), is a ˜50 kDa immunoglobulin superfamily membrane glycoprotein that is overexpressed in numerous blood cancers and solid tumors. High CD47 expression often correlates with more aggressive diseases and poorer clinical outcomes.

CD47 on the surface of CD47+ cells interacts with signal regulatory protein alpha (SIRPα) expressed on cells of the innate and adaptive immune systems, such as macrophages and dendritic cells. This interaction sends a “don't eat me” signal that inhibits phagocytosis, thereby allowing CD47+ cells to evade immune surveillance.

These data suggest that CD47 may serve as an immune checkpoint and that blocking the CD47-SIRPα interaction could have therapeutic value by switching off the “don't eat me” signal. Thus, blocking CD47 has emerged as a promising therapeutic strategy with numerous studies showing that interrupting the CD47-SIRPα signaling pathway promotes anti-tumor activity against human cancers in vitro and in vivo.

Several anti-CD47 monoclonal antibodies (mAbs) have been shown to increase phagocytosis of acute myeloid leukemia cells, non-Hodgkin's lymphoma cells, breast cancer cells, and ovarian cancer cells. In clinical studies, CD47 mAbs enhanced the anti-tumor activity of other therapeutic antibodies. At least six anti-CD47 mAbs and three SIRPα fusion proteins are in active phase I or II clinical trials for the treatment of human hematological malignancies and solid tumors.

The efficacy of anti-CD47 mAbs is limited by their interactions with red blood cells (RBCs), which also express CD47. RBCs act as a sink to sequester anti-CD47 antibodies, thereby preventing them from binding to malignant CD47-expressing (CD47+) cells. Furthermore, anti-CD47 mAb binding to RBCs leads to hemagglutination and lysis of the RBCs, resulting in anemia. Thus, there is a need for improved methods of treating malignant diseases mediated by CD47+ cells with reduced off-tumor effects.

Epithelial cell adhesion molecule (EpCAM), also known as CD236, is a ˜35 kDa type I transmembrane glycoprotein which functions as a homophilic Ca2+-independent cell-cell adhesion molecule. EpCAM is also involved in cell signaling, proliferation, differentiation, formation and maintenance of organ morphology. Its overexpression is found on many metastasizing epithelial cancers, such as breast, prostate, ovarian, lung, colon, renal and gastric cancers, highlighting its potential as an ideal target for immunotherapies (Spizzo et. al, Breast Cancer Res Treat 86: 207-213).

Epidermal growth factor receptor (EGFR), also known as ErbB-1 or HER1, is a transmembrane protein that is a receptor for members of the epidermal growth factor family of extracellular protein ligands. EGFR is a member of the ErbB family of receptors, a subfamily of four closely related receptor tyrosine kinases: EGFR, HER2/neu, Her 3, and Her4. Overexpression of EGFR has been associated with a number of cancers, including adenocarcinoma of the lung, anal cancers, glioblastoma, and epithelian tumors of the head and neck.

In some embodiments, disclosed herein are anti-CD47 antibodies, anti-EpCAM antibodies, and multi-specific antibodies which comprise a targeting moiety to CD47 and a targeting moiety to EpCAM, or a combination thereof. In some embodiments, also described herein are bispecific antibodies which comprise a first targeting moiety to CD47 and a second targeting moiety to EpCAM. Additional embodiments, further described herein, are methods of treating a cancer with an anti-CD47 antibody, an anti-EpCAM antibody, and a multi-specific antibody (e.g. a bispecific CD47/EpCAM antibody).

In some embodiments, disclosed herein are anti-CD47 antibodies, anti-EGFR antibodies, and multi-specific antibodies which comprise a targeting moiety to CD47 and a targeting moiety to EGFR or a combination thereof. In some embodiments, also described herein are bispecific antibodies which comprise a first targeting moiety to CD47 and a second targeting moiety to EGFR. Additional embodiments, further described herein, are methods of treating a cancer with an anti-CD47 antibody, an anti-EGFR antibody, and a multi-specific antibody (e.g. a bispecific CD47/EGFR antibody).

### 4.1 ANTI-CD47 ANTIBODIES

In certain embodiments, disclosed herein is an anti-CD47 antibody. In some embodiments, an anti-CD47 antibody described herein is a full-length antibody, comprising a heavy chain (HC) and a light chain (LC). In some cases, the HC comprises a sequence selected from Table 5. In some cases, the light chain comprises a sequence selected from Table 5. In some instances, the anti-CD47 antibody comprises a variable heavy chain (VH) region and a variable light chain (VL) region, wherein the VH region comprises CDR1 sequence selected from SEQ ID NO: 15; CDR2 sequence selected from SEQ ID NO: 16 and CDR3 sequence selected from SEQ ID NOs: 17; and wherein the VL region comprises CDR1, CDR2, and CDR3 sequences SEQ ID NOs 18, 19, and 20 respectively.

In some embodiments, the VH-CDR3 and/or VL-CDR3 comprises one or more amino acid substitutions that modify the affinity of binding to human CD47. In some cases, the amino acid substitution is an alanine (A). In some cases, the VH-CDR3 comprises amino acid substitutions at K₉₄, E₉₅, G₉₆, S₉₇, F₉₈, G₉₉, E₁₀₀, G₁₀₀ₐ, V_{100b}, D₁₀₁, and P₁₀₂. In one case, the VH-CDR3 comprises at least one alanine substitution selected from a group consisting of amino acid positions: K₉₄, E₉₅, G₉₆, S₉₇, F₉₈, G₉₉, E₁₀₀, G₁₀₀ₐ, V_{100b}, D₁₀₁, and P₁₀₂. In other cases, the VL-CDR3 comprises amino acid substitutions at positions Y₈₉, S₉₀, T₉₁, D₉₂, I₉₃, S₉₄, G₉₅, N₉₅ₐ, H_{95b}, W₉₆, and V₉₇. In one case, the VL-CDR3 at least one alanine substitution selected from a group consisting of amino acid positions: Y₈₉, S₉₀, T₉₁, D₉₂, I₉₃, S₉₄, G₉₅, N₉₅ₐ, H_{95b}, W₉₆, and V₉₇.

In some embodiments, the anti-CD47 antibody comprises a VH region in which the sequence of the VH region comprises about 80%, 85%, 90%, 95%, 96% 97%, 98%, 99%, or 100% sequence identity to SEQ ID NOs: 5, 10, and 13.

In some embodiments, an anti-CD47 antibody is modified at one or more amino acid positions to modify its binding, immunogenic, or other properties. In some cases, the mutations are in the heavy chain. In other cases, the mutations are in the light chain. In some cases, the mutations are in the heavy and light chains. In some embodiments, the modified amino acid residues of the heavy chain are selected from a group consisting of 167, F79 and G82b. In some embodiments, the modified amino acid residues of the light chain are selected from a group consisting of E₁₀₃ and M4. In some cases, the heavy chain comprises at least one mutation selected from a group consisting of I₆₇F, F₇₉Y, G_{82b}S. In some cases, the light chain comprises at least one mutation selected from a group consisting of E₁₀₃K and M₄L. In some embodiments, the heavy chain comprises the mutations I₆₇F, F₇₉Y, G_{82b}S. In some embodiments, the light chain comprises the mutations E₁₀₃K and M₄L.

In some embodiments, an anti-CD47 antibody is a full-length antibody. In other embodiments, the anti-CD47 antibody is a binding fragment. In some instances, the anti-CD47 antibody comprises an antibody or binding fragment thereof, a monoclonal antibody or binding fragment thereof, a chimeric antibody or binding fragment thereof, a humanized antibody or binding fragment thereof, or an engineered antibody or binding fragment thereof. In some cases, the anti-CD47 antibody comprises a monovalent Fab, a bivalent Fab′2, a single-chain variable fragment (scFv), or binding fragment thereof.

In some embodiments, an anti-CD47 antibody described herein has an EC50 of from about 0.02 nM to about 2.27 nM. In some embodiments, an anti-CD47 antibody described herein has an EC50 of from at least about 0.02 nM. In some embodiments, an anti-CD47 antibody described herein has an EC50 of from at most about 2.27 nM. In some embodiments, an anti-CD47 antibody described herein has an EC50 of from about 0.02 nM to about 0.16 nM, about 0.02 nM to about 0.21 nM, about 0.02 nM to about 1.03 nM, about 0.02 nM to about 1.2 nM, about 0.02 nM to about 2.27 nM, about 0.16 nM to about 0.21 nM, about 0.16 nM to about 1.03 nM, about 0.16 nM to about 1.2 nM, about 0.16 nM to about 2.27 nM, about 0.21 nM to about 1.03 nM, about 0.21 nM to about 1.2 nM, about 0.21 nM to about 2.27 nM, about 1.03 nM to about 1.2 nM, about 1.03 nM to about 2.27 nM, or about 1.2 nM to about 2.27 nM, in an in vitro phagocytosis assay to determine ADCP activity, for instance, using macrophages and targeting cancer cells such as metastatic cancer cells from a pancreatic cancer, for example, CFPAC-1 cells or from ovarian cancer, for example, OVISE cells.

In some embodiments, an anti-CD47 antibody described herein has an EC50 of from about 0.002 nM to about 0.138 nM. In some embodiments, an anti-CD47 antibody described herein has an EC50 of from at least about 0.002 nM. In some embodiments, an anti-CD47 antibody described herein has an EC50 of from at most about 0.138 nM. In some embodiments, an anti-CD47 antibody described herein has an EC50 of from about 0.002 nM to about 0.012 nM, about 0.002 nM to about 0.053 nM, about 0.002 nM to about 0.108 nM, about 0.002 nM to about 0.138 nM, about 0.012 nM to about 0.053 nM, about 0.012 nM to about 0.108 nM, about 0.012 nM to about 0.138 nM, about 0.053 nM to about 0.108 nM, about 0.053 nM to about 0.138 nM, or about 0.108 nM to about 0.138 nM in an in vitro cytotoxicity assay to determine ADCC activity, for instance, using NK92/CD16a176V effector cells and targeting cancer cells such as metastatic cancer cells from a pancreatic cancer, for example, CFPAC-1 cells or from ovarian cancer, for example, OVISE cells.

In some embodiments, an anti-CD47 antibody described herein has decreased red blood cell (RBC) binding compared to CD47 BMK-1. In some cases, the decreased RBC binding is a decrease of at least 10%, 20%, 30%, 40%, 50%, 60%, 70%, 80%, 90%, 95%, or 100% compared to reference antibody CD47 BMK-1.

In some embodiments, an anti-CD47 antibody described herein has an improved serum half-life compared to reference antibody CD47 BMK-2, CD47 BMK-1 or CD47 BMK-4. In some embodiments, an anti-CD47 antibody described herein has an improved serum half-life compared to reference antibody CD47 BMK-2 or CD47 BMK-1. In some instances, the improved serum half-life is at least 30 minutes, 1 hour, 1.5 hour, 2 hours, 3 hours, 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 14 days, 30 days, or longer than reference antibody CD47 BMK-2, CD47 BMK-1 or CD47 BMK-4. In some instances, the improved serum half-life is at least 30 minutes, 1 hour, 1.5 hour, 2 hours, 3 hours, 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 14 days, 30 days, or longer than reference antibody CD47 BMK-2 or CD47 BMK-1.

In some cases, the serum half-life of an anti-CD47 antibody described herein is at least 30 minutes, 1 hour, 1.5 hour, 2 hours, 3 hours, 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 14 days, 30 days, or longer. In some cases, the serum half-life of an anti-CD47 antibody described herein is about 30 minutes, 1 hour, 1.5 hour, 2 hours, 3 hours, 4 hours, 5 hours, 6 hours, 7 hours, 8 hours, 9 hours, 10 hours, 12 hours, 18 hours, 24 hours, 2 days, 3 days, 4 days, 5 days, 6 days, 7 days, 14 days, 30 days, or longer.

### 4.2 ANTI-EPCAM ANTIBODIES

In certain embodiments, disclosed herein is an anti-EpCAM antibody. In some embodiments, an anti-EpCAM antibody described herein is a full-length antibody, comprising a heavy chain (HC) and a light chain (LC). In some cases, the HC comprises a sequence selected from Table 8. In some cases, the light chain comprises a sequence selected from Table 9. In some instances, the anti-EpCAM antibody comprises a variable heavy chain (VH) region and a variable light chain (VL) region, wherein the VH region comprises CDR1 sequence selected from SEQ ID NOs: 45, 48, 51, 54, 57, 60, 63, 66, 69, 72, 75, 78; CDR2 sequence selected from SEQ ID NOs: 46, 49, 52, 55, 58, 61, 64, 67, 70, 73, 76, 79 and CDR3 sequence selected from SEQ ID NOs: 47, 50, 53, 56, 59, 62, 65, 68, 71, 74, 77, 80 and wherein the VL region comprises CDR1 sequence selected from SEQ ID NOs: 81, 84, 87, 90, 93, 96, 99, 102, 105, 108, 111, and 114; CDR2 sequence selected from SEQ ID NOs: 82, 85, 88, 91, 94, 97, 100, 103, 106, 109, 112, and 115; and CDR3 sequence selected from SEQ ID NOs: 83, 86, 89, 92, 95, 98, 101, 104, 107, 110, 113, and 116.

In some embodiments, a VH-CDR3 and/or VL-CDR3 comprises one or more amino acid substitutions that modify binding to human EpCAM, immunogenicity, or some other feature. In some cases, the amino acid substitution is an alanine (A). In some embodiments, the VH-CDR3 is SEQ ID NO: 47, 50, 53, 56, 59, 62, 65, 68, 71, 74, 77, 80. In some embodiments, the VH-CDR3 comprises at least one alanine substitution. In some embodiments, the alanine substitution ablates binding to human EpCAM.

In some embodiments, the anti-EpCAM antibody comprises a VH region and a VL region in which the sequence of the VH region comprises about 80%, 85%, 90%, 95%, 96% 97%, 98%, 99%, or 100% sequence identity to SEQ ID NOs: 21-32 and the sequence of the VL region comprises about 80%, 85%, 90%, 95%, 96% 97%, 98%, 99%, or 100% sequence identity to SEQ ID NOs: 33-44.

In some embodiments, an anti-EpCAM antibody is a full-length antibody. In other embodiments, the anti-EpCAM antibody is a binding fragment. In some instances, the anti-EpCAM antibody comprises an antibody or binding fragment thereof, a monoclonal antibody or binding fragment thereof, a chimeric antibody or binding fragment thereof, or a humanized antibody or binding fragment thereof. In some cases, the anti-EpCAM antibody comprises a monovalent Fab, a bivalent Fab′2, a single-chain variable fragment (scFv), or binding fragment thereof.

In some embodiments, an anti-EpCAM antibody described herein has enhanced binding to human EpCAM protein compared to the reference antibody, EpCAM BMK-6. In some cases, the enhance binding is a decrease in KD of at least 10%, 20%, 30%, 40%, 50%, 60%, 70%, 80%, 90%, 100% compared to EpCAM BMK-6.

In some embodiments, an anti-EpCAM antibody described herein has an EC50 of from about 0.023 nM to about 3.07 nM. In some embodiments, an anti-EpCAM antibody described herein has an EC50 of from at least about 0.023 nM. In some embodiments, an anti-EpCAM antibody described herein has an EC50 of from at most about 3.07 nM. In some embodiments, an anti-EpCAM antibody described herein has an EC50 of from about 0.023 nM to about 0.024 nM, about 0.023 nM to about 0.036 nM, about 0.023 nM to about 0.038 nM, about 0.023 nM to about 0.04 nM, about 0.023 nM to about 0.059 nM, about 0.023 nM to about 0.069 nM, about 0.023 nM to about 0.086 nM, about 0.023 nM to about 3.07 nM, about 0.024 nM to about 0.036 nM, about 0.024 nM to about 0.038 nM, about 0.024 nM to about 0.04 nM, about 0.024 nM to about 0.059 nM, about 0.024 nM to about 0.069 nM, about 0.024 nM to about 0.086 nM, about 0.024 nM to about 3.07 nM, about 0.036 nM to about 0.038 nM, about 0.036 nM to about 0.04 nM, about 0.036 nM to about 0.059 nM, about 0.036 nM to about 0.069 nM, about 0.036 nM to about 0.086 nM, about 0.036 nM to about 3.07 nM, about 0.038 nM to about 0.04 nM, about 0.038 nM to about 0.059 nM, about 0.038 nM to about 0.069 nM, about 0.038 nM to about 0.086 nM, about 0.038 nM to about 3.07 nM, about 0.04 nM to about 0.059 nM, about 0.04 nM to about 0.069 nM, about 0.04 nM to about 0.086 nM, about 0.04 nM to about 3.07 nM, about 0.059 nM to about 0.069 nM, about 0.059 nM to about 0.086 nM, about 0.059 nM to about 3.07 nM, about 0.069 nM to about 0.086 nM, about 0.069 nM to about 3.07 nM, or about 0.086 nM to about 3.07 nM in an in vitro cytotoxicity assay to determine ADCC activity, for instance, using NK92/CD16a176V effector cells and targeting cancer cells such as metastatic cancer cells from a vulvar squamous cell carcinoma, for example, A431 cells.

In some embodiments, a first anti-EpCAM antibody described herein inhibits binding of a second anti-EpCAM antibody or fragment thereof, to human EpCAM protein. In some embodiments, the first anti-EpCAM antibody inhibits binding by 0%, 10%, 20%, 30%, 40%, 50%, 60%, 70%, 80%, 95%, 100%.

### 4.3 ANTI-EGFR ANTIBODIES

In certain embodiments, disclosed herein is an anti-EGFR antibody. In some embodiments, an anti-EGFR antibody described herein is a full-length antibody, comprising a heavy chain (HC) and a light chain (LC). In some cases, the HC comprises a sequence selected from Tables 15-17. In some cases, the light chain comprises a sequence selected from Tables 15-17. In some instances, the anti-EGFR antibody comprises a variable heavy chain (VH) region and a variable light chain (VL) region, wherein the VH region comprises CDR1 according to SEQ ID NO: 121; CDR2 sequence according to SEQ ID NO: 122 and CDR3 according to SEQ ID NO: 123 and wherein the VL region comprises CDR1, CDR2, and CDR3 sequences SEQ ID NOs: 124, 125, and 126 respectively.

In some embodiments, a VH-CDR3 and/or VL-CDR3 comprises one or more amino acid substitutions that modify binding to human EGFR, immunogenicity, or some other feature. In some cases, the amino acid substitution is an alanine (A). In some embodiments, the VH-CDR3 comprises at least one alanine substitution. In some embodiments, the alanine substitution ablates binding to human EGFR.

In some embodiments, the anti-EGFR antibody comprises a VH region and a VL region in which the sequence of the VH region comprises about 80%, 85%, 90%, 95%, 96% 97%, 98%, 99%, or 100% sequence identity to SEQ ID NO: 127 and the sequence of the VL region comprises about 80%, 85%, 90%, 95%, 96% 97%, 98%, 99%, or 100% sequence identity to SEQ ID NO: 128.

In some embodiments, an anti-EGFR antibody is a full-length antibody. In other embodiments, the anti-EGFR antibody is a binding fragment. In some instances, the anti-EGFR antibody comprises an antibody or binding fragment thereof, a monoclonal antibody or binding fragment thereof, a chimeric antibody or binding fragment thereof, or a humanized antibody or binding fragment thereof. In some cases, the anti-EGFR antibody comprises a monovalent Fab, a bivalent Fab′2, a single-chain variable fragment (scFv), or binding fragment thereof.

In some embodiments, an anti-EGFR antibody described herein has enhanced binding to human EGFR protein compared to a reference antibody. In some cases, the enhance binding is a decrease in KD of at least 10%, 20%, 30%, 40%, 50%, 60%, 70%, 80%, 90%, 100% compared to reference antibody.

In some embodiments, an anti-EGFR antibody described herein has an EC50 of from about 0.023 nM to about 3.07 nM. In some embodiments, an anti-EGFR antibody described herein has an EC50 of from at least about 0.023 nM. In some embodiments, an anti-EGFR antibody described herein has an EC50 of from at most about 3.07 nM. In some embodiments, an anti-EGFR antibody described herein has an EC50 of from about 0.023 nM to about 0.024 nM, about 0.023 nM to about 0.036 nM, about 0.023 nM to about 0.038 nM, about 0.023 nM to about 0.04 nM, about 0.023 nM to about 0.059 nM, about 0.023 nM to about 0.069 nM, about 0.023 nM to about 0.086 nM, about 0.023 nM to about 3.07 nM, about 0.024 nM to about 0.036 nM, about 0.024 nM to about 0.038 nM, about 0.024 nM to about 0.04 nM, about 0.024 nM to about 0.059 nM, about 0.024 nM to about 0.069 nM, about 0.024 nM to about 0.086 nM, about 0.024 nM to about 3.07 nM, about 0.036 nM to about 0.038 nM, about 0.036 nM to about 0.04 nM, about 0.036 nM to about 0.059 nM, about 0.036 nM to about 0.069 nM, about 0.036 nM to about 0.086 nM, about 0.036 nM to about 3.07 nM, about 0.038 nM to about 0.04 nM, about 0.038 nM to about 0.059 nM, about 0.038 nM to about 0.069 nM, about 0.038 nM to about 0.086 nM, about 0.038 nM to about 3.07 nM, about 0.04 nM to about 0.059 nM, about 0.04 nM to about 0.069 nM, about 0.04 nM to about 0.086 nM, about 0.04 nM to about 3.07 nM, about 0.059 nM to about 0.069 nM, about 0.059 nM to about 0.086 nM, about 0.059 nM to about 3.07 nM, about 0.069 nM to about 0.086 nM, about 0.069 nM to about 3.07 nM, or about 0.086 nM to about 3.07 nM in an in vitro cytotoxicity assay to determine ADCC activity, for instance, using NK92/CD16a176V effector cells and targeting cancer cells such as metastatic cancer cells from a vulvar squamous cell carcinoma, for example, A431 cells.

In some embodiments, a first anti-EGFR antibody described herein inhibits binding of a second anti-EGFR antibody or fragment thereof, to human EGFR protein. In some embodiments, the first anti-EGFR antibody inhibits binding by 0%, 10%, 20%, 30%, 40%, 50%, 60%, 70%, 80%, 95%, 100%.

### 4.4 BISPECIFIC ANTIBODIES

In some embodiments, a bispecific antibody comprises a first targeting moiety specific for CD47 and a second targeting moiety specific to a tumor cell. In some embodiments, the second target moiety is specific for an antigen expressed by a tumor cell. In some cases, the antigen expressed by a tumor cell is a tumor-associated antigen (TAA). In some embodiments, the tumor-associated antigen includes but is not limited to: ACVR2, HER2/neu, CD20, EGFR, CD3, CD22, CD80, CD23, EpCAM, CD2, CD3, CD19, mesothelin, Mum-1, β-catenin, CDK4, p53, Ras, CDC27, α-actinin-4, TRP1/gp75, Wilm, EphA3, prostatic acid phosphatase (PAP), alpha-fetoprotein (AFP), 9D7, Cyclin-B₁, carcinoembryonic antigen (CEA), gp100/pmel17, BRCA1/2, VEGFR, MUC-1, epithelial tumor antigen (ETA), tyrosinase, melanoma associated antigen (MAGE), carbonic anhydrase IX, cytotoxic T-lymphocyte antigen 4, Folate-Binding Protein A-33, prostate specific antigen (PSA), survivin, EGFRvIII, melanocyte derived peptide, multiple melanoma-associated peptides, cervical carcinoma antigen HPV-16-E7, PRAME, SSX-2, CA125, MART, CS-1, BING-4, fibronectin, CML66, MC1R, calcium-activated chloride channel 2, immature laminin receptor, and hTERT. In certain embodiments, the TAA is expressed on the cell surface.

In certain embodiments, described herein is a bispecific anti-CD47 and anti-EpCAM antibody, or binding fragment thereof. In some instances, the bispecific anti-CD47 and anti-EpCAM antibody comprises at least one targeting moiety that specifically binds to CD47 and at least one targeting moiety that specifically binds to EpCAM. In some instances, the bispecific antibody is bivalent, trivalent, tetravalent or more than tetravalent. In some instances, the bispecific antibody has more than one binding site that binds to CD47. In some instances, the bispecific antibody has more than one binding site that binds to EpCAM. In some instances, the bispecific antibody or binding fragment thereof is a bispecific antibody conjugate, a hybrid bispecific IgG, a variable domain only bispecific antibody, a CH1/CL fusion protein, a Fab fusion protein, a non-immunoglobulin fusion protein, a Fc-modified IgG, an appended & Fc-modified IgG, a modified Fc and CH3 fusion protein, an appended IgG-HC fusion, a Fc fusion, a CH3 fusion, an IgE/IgM CH2 fusion, or a F(ab′)2 fusion.

In certain embodiments, described herein is a bispecific anti-CD47 and anti-EGFR antibody, or binding fragment thereof. In some instances, the bispecific anti-CD47 and anti-EGFR antibody comprises at least one targeting moiety that specifically binds to CD47 and at least one targeting moiety that specifically binds to EGFR. In some instances, the bispecific antibody is bivalent, trivalent, tetravalent or more than tetravalent. In some instances, the bispecific antibody has more than one binding site that binds to CD47. In some instances, the bispecific antibody has more than one binding site that binds to EGFR. In some instances, the bispecific antibody or binding fragment thereof is a bispecific antibody conjugate, a hybrid bispecific IgG, a variable domain only bispecific antibody, a CH1/CL fusion protein, a Fab fusion protein, a non-immunoglobulin fusion protein, a Fc-modified IgG, an appended & Fc-modified IgG, a modified Fc and CH3 fusion protein, an appended IgG-HC fusion, a Fc fusion, a CH3 fusion, an IgE/IgM CH2 fusion, or a F(ab′)2 fusion.

In some cases, the bispecific antibody further comprises one or more mutations in a framework region, e.g., in the CH1 domain, CH2 domain, CH3 domain, hinge region, or a combination thereof. In some instances, the one or more mutations are to stabilize the antibody and/or to increase half-life. In some instances, the one or more mutations are to modulate Fc receptor interactions, to increase ADCC or antibody-dependent cellular phagocytosis (ADCP). In other instances, the one or more mutations are to reduce or eliminate Fc effector functions such as FcγR-binding, ADCC or ADCP. In additional instances, the one or more mutations are to modulate glycosylation. In some cases, the one or more mutations enhance stability, increase half-life, decrease glycosylation, and/or modulate Fc receptor interactions, e.g., to increase or decrease ADCC and/or ADCP.

In some cases, the bispecific antibody comprises an IgG1 framework. In some embodiments, the constant region of the anti-CD47 or anti-EpCAM or EGFR antibody is modified at one or more amino acid positions to alter Fc receptor interaction. Exemplary residues that modulate or alter Fc receptor interaction include, but are not limited to, G236, S239, T250, M252, S254, T256, K326, A330, 1332, E333A, M428, H433, or N434 (Kabat numbering; EU index of Kabat et al 1991 Sequences of Proteins of Immunological Interest). In some instances, the mutation comprises G236A, S239D, T250Q, M252Y, S254T, T256E, K326W, A330L, 1332E, E333A, E333S, M428L, H433K, or N434F.

In some embodiments, the modification at one or more amino acid positions in the IgG1 constant region to alter Fc receptor interaction leads to increased half-life. In some instances, the modification at one or more amino acid positions comprise T250, M252, S254, T256, M428, H433, N434, or a combination thereof; e.g., comprising T250Q/M428L or M252Y/S254T/T256E and H433K/N434F.

In some embodiments, a bispecific antibody described above comprises a knobs-into-holes (KIH) format. In some cases, the KIH is located in the Fc region, in which the residues within the CH3 domain are optionally modified based on the disclosure of WO96/027011; Ridgway, et. al., Protein Eng. 9 (1996) 617-621; or Merchant, et. al., Nat. Biotechnol. 16 (1998) 677-681. In some cases, one of the CH3 domain pair is the “knob” chain while the other is the “hole” chain and additional disulfide bridges are optionally introduced to further stabilize the antibody and/or to increase yield.

In some instances, the bispecific antibody is an IgG1, and the CH3 domain of the “knob” chain comprises a T366W mutation and the CH3 domain of the “hole” chain comprises mutations T366S, L368A, and Y407V. In some cases, the CH3 domain of the “knob” chain further comprises a Y349C mutation which forms an interchain disulfide bridge with either E356C or S354C in the CH3 domain of the “hole” chain.

In some instances, the CH3 domain of the “knob” chain comprises R409D and K370E mutations and the CH3 domain of the “hole” chain comprises D399K and E357K. In some cases, the CH3 domain of the “knob” chain further comprises a T366W mutation and the CH3 domain of the “hole” chain further comprises mutations T366S, L368A, and Y407V.

In some embodiments, modification at one or more amino acid positions in the IgG1 constant region to alter Fc receptor interaction leads to increased ADCC and/or ADCP. In some instances, the modification at one or more amino acid positions comprises S239, K326, A330, 1332, E333, or a combination thereof. In some instances, the modification at one or more amino acid positions for increased ADCC and/or ADCP comprises, e.g., E333A, S239D/A330L/1332E, or K326W/E333S. In some cases, the modification at one or more amino acid positions for increased ADCC comprises S239D/A330L/1332E. In some cases, the modification at one or more amino acid positions for increased ADCP comprises K326W/E333S.

In some embodiments, the IgG1 constant region is afucosylated. In other embodiments, the IgG1 is expressed in cells incapable of fucosylation. In some embodiments, the cell is a mammalian cell such as a Chinese Hamster Ovary cell line. In some embodiments, the cell is unable to express fucosyltransferase 8 (FUT8).

In some embodiments, the bispecific antibody comprises an IgG2 framework. In some instance, one or more amino acid positions in the IgG2 framework are modified to alter Fc receptor interaction, e.g., to increase ADCC and/or CDC. In some cases, one or more amino acid positions in the IgG2 framework are modified to stabilize the antibody and/or to increase half-life. In some instances, one or more amino acid positions in the IgG2 framework are modified to modulate glycosylation. In some cases, the IgG2 constant region is afucosylated. In some embodiments, the IgG2 constant region is expressed in cells incapable of fucosylation. In some embodiments, the cell is a mammalian cell such as a Chinese Hamster Ovary cell line. In some embodiments, the cell is unable to express fucosyltransferase 8 (FUT8).

In some embodiments, the bispecific antibody comprises an IgG3 framework. In some instance, one or more amino acid positions in the IgG3 framework are modified to alter Fc receptor interaction, e.g., to increase ADCC and/or ADCP. In some cases, one or more amino acid positions in the IgG3 framework are modified to stabilize the antibody and/or to increase half-life. In some instances, one or more amino acid positions in the IgG3 framework are modified to modulate glycosylation. In some cases, the constant region of the antibody is modified at amino acid R435 to extend the half-life, e.g., R435H (Kabat numbering). In some instances, the constant region is deglycosylated at residue N297. In some instances, the IgG3 constant region is expressed in cells incapable of fucosylation. In some embodiments, the cell is a mammalian cell such as a Chinese Hamster Ovary cell line. In some embodiments, the cell is unable to express fucosyltransferase 8 (FUT8).

In some embodiments, the bispecific antibody comprises an IgG4 framework. In some instance, one or more amino acid positions in the IgG4 framework are modified to alter Fc receptor interaction, e.g., to increase ADCC and/or ADCP. For example, mutations to increase ADCC comprises, in some embodiments, S239D, 1332E, and A330L (amino acid numbering is according to the EU index in Kabat et al), such as described in U.S. Pat. No. 8,093,359. In some cases, one or more amino acid positions in the IgG4 framework are modified to stabilize the antibody and/or to increase half-life. In some instances, one or more amino acid positions in the IgG4 framework are modified to modulate glycosylation. In some cases, the constant region is modified at a hinge region to prevent or reduce strand exchange. In some instances, the amino acid that is modified is S228 (e.g., S228P). In some instances, the IgG4 constant region is expressed in cells incapable of fucosylation. In some embodiments, the cell is a mammalian cell such as a Chinese Hamster Ovary cell line. In some embodiments, the cell is unable to express fucosyltransferase 8 (FUT8).

In some embodiments, the human IgG constant region is modified to alter its ADCC and/or ADCP activity, e.g., with an amino acid modification described in Natsume et al., 2008 Cancer Res, 68(10): 3863-72; Idusogie et al., 2001 J Immunol, 166(4): 2571-5; Moore et al., 2010 mAbs, 2(2): 181-189; Lazar et al., 2006 PNAS, 103(11): 4005-4010, Shields et al., 2001 JBC, 276(9): 6591-6604; Stavenhagen et al., 2007 Cancer Res, 67(18): 8882-8890; Stavenhagen et al., 2008 Advan. Enzyme Regul., 48: 152-164; Alegre et al, 1992 J Immunol, 148: 3461-3468; Reviewed in Kaneko and Niwa, 2011 Biodrugs, 25(1): 1-11.

In some embodiments, the human IgG constant region is modified to induce heterodimerization. For example, having an amino acid modification within the CH3 domain at Thr366, which when replaced with a more bulky amino acid, e.g., Trp (T366W), is able to preferentially pair with a second CH3 domain having amino acid modifications to less bulky amino acids at positions Thr366, Leu368, and Tyr407, e.g., Ser, Ala and Val, respectively (T366S/L368A/Y407V). In some cases, heterodimerization via CH3 modifications is further stabilized by the introduction of a disulfide bond, for example by changing Ser354 to Cys (S354C) and Y349 to Cys (Y349C) on opposite CH3 domains (Reviewed in Carter, 2001 Journal of Immunological Methods, 248: 7-15).

In some instances, a bispecific antibody described herein has reduced or lacks glycosylation but is not modified at amino acid Asn297 (Kabat numbering). In these instances, the glycosylation is, for example, eliminated by production of the antibody in a host cell that lacks a post-translational glycosylation capacity, for example a bacterial or yeast derived system or a modified mammalian cell expression system. In some embodiments, the cell is a mammalian cell such as a Chinese Hamster Ovary cell line. In some embodiments, the cell is unable to express fucosyltransferase 8 (FUT8). In certain aspects, such a system is a cell-free expression system.

In some embodiments, the bispecific protein comprising a CD47-binding first component and an EpCAM-binding second component have different affinities (KD) for their respective target antigens as measured by surface plasmon resonance.

In some cases, the first component binds to human CD47 with a KD of from about 0.1 nM to about 100 nM, from about 0.15 nM to about 95 nM, from about 0.2 nM to about 90 nM, from 0.25 nM to about 85 nM, from about 0.3 nM to about 80 nM, from about 0.35 nM to about 75 nM, from about 0.4 nM to about 70 nM, from about 0.5 nM to about 70 nM, from about 0.6 nM to about 60 nM, from about 0.7 nM to about 50 nM, from about 0.8 nM to about 40 nM, from about 0.9 nM to about 30 nM, from about 1 nM to about 20 nM, from about 1.5 nM to about 10 nM, from about 0.01 nM to about 25 nM, from about 0.01 nM to about 20 nM, from about 0.01 nM to about 10 nM, from about 0.01 nM to about 5 nM, from about 0.02 nM to about 20 nM, from about 0.04 nM to about 20 nM, from about 0.06 nM to about 20 nM, from about 0.08 nM to about 20 nM, or from about 0.1 nM to about 20 nM.

In some cases, the second component binds to human EpCAM with a KD of about 0.1 nM to about 500 nM, from about 0.2 nM to about 500 nM, from about 1 nM to about 300 nM, from about 5 nM to about 200 nM, or from about 10 nM to about 150 nM

### 4.5 HUMANIZATION

In some embodiments, the bispecific antibodies, and binding fragments thereof, are derived from non-human (e.g. rabbit or mouse) antibodies. In some instances, the humanized form of the non-human antibody contains a minimal non-human sequence to maintain original antigenic specificity. In some cases, the humanized antibodies are human immunoglobulins (acceptor antibody), wherein the CDRs of the acceptor antibody are replaced by residues of the CDRs of a non-human immunoglobulin (donor antibody), such as rat, rabbit, or mouse donor having the desired specificity, affinity, avidity, binding kinetics, and/or capacity. In some instances, one or more framework region (FR) residues of the human immunoglobulin are replaced by corresponding non-human residues of the donor antibody.

### 4.6 BISPECIFIC ANTIBODY BINDING TO TARGET CELLS

In some embodiments, a bispecific antibody of the present disclosure that comprises a first component that binds CD47 and a second component that binds EpCAM, binds to a cell that expresses on its surface target antigens of the bispecific protein, with at least 2-50 fold, 10-100 fold, 2-fold, 5-fold, 10-fold, 25-fold, 50-fold or 100-fold, or 20-50%, 50-100%, 20%, 25%, 30%, 40%, 50%, 60%, 70%, 80%, 90% or 100% or more higher affinity (e.g., preferentially binds) compared to the binding affinity of an antibody that is bivalent to only one of CD47 or EpCAM, to the cell.

In some embodiments, a bispecific protein provided herein binds to a target cell that expresses a higher level of EpCAM than CD47, on its surface. For instance, the ratio of EpCAM to CD47 protein expression on the target cell surface, is from about 1, 1.5, 2.0, 2.5, 5, 10, 15, 20, 25, 50, 100, or greater than 200 as measured by flow cytometry.

In some embodiments, a bispecific protein provided herein binds to a target cell that expresses a higher level of CD47 than EpCAM, on its surface. For instance, the ratio of CD47 to EpCAM protein expression on the target cell surface, is from about 1, 1.5, 2.0, 2.5, 5, 10, 15, 20, 50, 100, or greater than 200 as measured by flow cytometry.

In some embodiments, a bispecific protein provided herein binds to a target cell that expresses equal levels of CD47 and EpCAM, on its surface. For instance, the ratio of CD47 to EpCAM protein expression on the target cell surface, is about 1 as measured by flow cytometry.

In some cases, a cell expresses about 35,000 CD47 proteins to about 250,000 CD47 proteins. In some cases, a cell expresses at least about 35,000 CD47 proteins. In some cases, a cell expresses at most about 250,000 CD47 proteins. In some cases, a cell expresses about 35,000 CD47 proteins to about 40,000 CD47 proteins, about 35,000 CD47 proteins to about 45,000 CD47 proteins, about 35,000 CD47 proteins to about 60,000 CD47 proteins, about 35,000 CD47 proteins to about 100,000 CD47 proteins, about 35,000 CD47 proteins to about 150,000 CD47 proteins, about 35,000 CD47 proteins to about 250,000 CD47 proteins, about 40,000 CD47 proteins to about 45,000 CD47 proteins, about 40,000 CD47 proteins to about 60,000 CD47 proteins, about 40,000 CD47 proteins to about 120,000 CD47 proteins, about 40,000 CD47 proteins to about 150,000 CD47 proteins, about 40,000 CD47 proteins to about 250,000 CD47 proteins, about 45,000 CD47 proteins to about 60,000 CD47 proteins, about 45,000 CD47 proteins to about 120,000 CD47 proteins, about 45,000 CD47 proteins to about 150,000 CD47 proteins, about 45,000 CD47 proteins to about 250,000 CD47 proteins, about 60,000 CD47 proteins to about 120,000 CD47 proteins, about 60,000 CD47 proteins to about 150,000 CD47 proteins, about 60,000 CD47 proteins to about 250,000 CD47 proteins, about 120,000 CD47 proteins to about 150,000 CD47 proteins, about 120,000 CD47 proteins to about 250,000 CD47 proteins, or about 150,000 CD47 proteins to about 250,000 CD47 proteins. In some cases, the CD47 proteins are expressed on the surface of a cell. In some cases, the cell is a tumor cell. In some cases, the cell is derived from a ductal pancreatic adenocarcinoma, an ovarian clear cell adenocarcinoma, a colon adenocarcinoma, a lung adenocarcinoma, an ovarian adeno carcinoma, or a vulvar squamous cell carcinoma. In some cases, the number of CD47 proteins expressed on a cell is measured by flow cytometry. In some cases, the number of CD47 proteins expressed on a cell is measured by quantitative flow cytometry.

In some cases, a cell expresses about 35,000 EpCAM proteins to about 2×10⁷ EpCAM proteins. In some cases, a cell expresses at least about 35,000 EpCAM proteins. In some cases, a cell expresses at most about 2×10⁷ EpCAM proteins. In some cases, a cell expresses about 35,000 EpCAM proteins to about 85,000 EpCAM proteins, about 35,000 EpCAM proteins to about 170,000 EpCAM proteins, about 35,000 EpCAM proteins to about 300,000 EpCAM proteins, about 35,000 EpCAM proteins to about 400,000 EpCAM proteins, about 35,000 EpCAM proteins to about 650,000 EpCAM proteins, about 35,000 EpCAM proteins to about 750,000 EpCAM proteins, about 35,000 EpCAM proteins to about 1,500,000 EpCAM proteins, about 35,000 EpCAM proteins to about 1,900,000 EpCAM proteins, about 35,000 EpCAM proteins to about 4×10⁶ EpCAM proteins, about 35,000 EpCAM proteins to about 6×10⁶ EpCAM proteins, about 35,000 EpCAM proteins to about 7×10⁶ EpCAM proteins, about 35,000 EpCAM proteins to about 15×10⁶ EpCAM proteins, about 35,000 EpCAM proteins to about 2×10⁷ EpCAM proteins, about 85,000 EpCAM proteins to about 170,000 EpCAM proteins, about 85,000 EpCAM proteins to about 300,000 EpCAM proteins, about 85,000 EpCAM proteins to about 400,000 EpCAM proteins, about 85,000 EpCAM proteins to about 650,000 EpCAM proteins, about 85,000 EpCAM proteins to about 750,000 EpCAM proteins, about 85,000 EpCAM proteins to about 1,500,000 EpCAM proteins, about 85,000 EpCAM proteins to about 1,900,000 EpCAM proteins, about 85,000 EpCAM proteins to about 4×10⁶ EpCAM proteins, about 85,000 EpCAM proteins to about 6×10⁶ EpCAM proteins, about 85,000 EpCAM proteins to about 7×10⁶ EpCAM proteins, about 85,000 EpCAM proteins to about 15×10⁶ EpCAM proteins, about 85,000 EpCAM proteins to about 2×10⁷ EpCAM proteins, about 170,000 EpCAM proteins to about 300,000 EpCAM proteins, about 170,000 EpCAM proteins to about 400,000 EpCAM proteins, about 170,000 EpCAM proteins to about 650,000 EpCAM proteins, about 170,000 EpCAM proteins to about 750,000 EpCAM proteins, about 170,000 EpCAM proteins to about 1,500,000 EpCAM proteins, about 170,000 EpCAM proteins to about 1,900,000 EpCAM proteins, about 170,000 EpCAM proteins to about 4×10⁶ EpCAM proteins, about 170,000 EpCAM proteins to about 6×10⁶ EpCAM proteins, about 170,000 EpCAM proteins to about 7×10⁶ EpCAM proteins, about 170,000 EpCAM proteins to about 15×10⁶ EpCAM proteins, about 170,000 EpCAM proteins to about 2×10⁷ EpCAM proteins, about 300,000 EpCAM proteins to about 400,000 EpCAM proteins, about 300,000 EpCAM proteins to about 650,000 EpCAM proteins, about 300,000 EpCAM proteins to about 750,000 EpCAM proteins, about 300,000 EpCAM proteins to about 1,500,000 EpCAM proteins, about 300,000 EpCAM proteins to about 1,900,000 EpCAM proteins, about 300,000 EpCAM proteins to about 4×10⁶ EpCAM proteins, about 300,000 EpCAM proteins to about 6×10⁶ EpCAM proteins, about 300,000 EpCAM proteins to about 7×10⁶ EpCAM proteins, about 300,000 EpCAM proteins to about 15×10⁶ EpCAM proteins, about 300,000 EpCAM proteins to about 2×10⁷ EpCAM proteins, about 400,000 EpCAM proteins to about 650,000 EpCAM proteins, about 400,000 EpCAM proteins to about 750,000 EpCAM proteins, about 400,000 EpCAM proteins to about 1,500,000 EpCAM proteins, about 400,000 EpCAM proteins to about 1,900,000 EpCAM proteins, about 400,000 EpCAM proteins to about 4×10⁶ EpCAM proteins, about 400,000 EpCAM proteins to about 6×10⁶ EpCAM proteins, about 400,000 EpCAM proteins to about 7×10⁶ EpCAM proteins, about 400,000 EpCAM proteins to about 15×10⁶ EpCAM proteins, about 400,000 EpCAM proteins to about 2×10⁷ EpCAM proteins, about 650,000 EpCAM proteins to about 750,000 EpCAM proteins, about 650,000 EpCAM proteins to about 1,500,000 EpCAM proteins, about 650,000 EpCAM proteins to about 1,900,000 EpCAM proteins, about 650,000 EpCAM proteins to about 4×10⁶ EpCAM proteins, about 650,000 EpCAM proteins to about 6×10⁶ EpCAM proteins, about 650,000 EpCAM proteins to about 7×10⁶ EpCAM proteins, about 650,000 EpCAM proteins to about 15×10⁶ EpCAM proteins, about 650,000 EpCAM proteins to about 2×10⁷ EpCAM proteins, about 750,000 EpCAM proteins to about 1,500,000 EpCAM proteins, about 750,000 EpCAM proteins to about 1,900,000 EpCAM proteins, about 750,000 EpCAM proteins to about 4×10⁶ EpCAM proteins, about 750,000 EpCAM proteins to about 6×10⁶ EpCAM proteins, about 750,000 EpCAM proteins to about 7×10⁶ EpCAM proteins, about 750,000 EpCAM proteins to about 15×10⁶ EpCAM proteins, about 750,000 EpCAM proteins to about 2×10⁷ EpCAM proteins, about 1,500,000 EpCAM proteins to about 1,900,000 EpCAM proteins, about 1,500,000 EpCAM proteins to about 4×10⁶ EpCAM proteins, about 1,500,000 EpCAM proteins to about 6×10⁶ EpCAM proteins, about 1,500,000 EpCAM proteins to about 7×10⁶ EpCAM proteins, about 1,500,000 EpCAM proteins to about 15×10⁶ EpCAM proteins, about 1,500,000 EpCAM proteins to about 2×10⁷ EpCAM proteins, about 1,900,000 EpCAM proteins to about 4×10⁶ EpCAM proteins, about 1,900,000 EpCAM proteins to about 6×10⁶ EpCAM proteins, about 1,900,000 EpCAM proteins to about 7×10⁶ EpCAM proteins, about 1,900,000 EpCAM proteins to about 15×10⁶ EpCAM proteins, about 1,900,000 EpCAM proteins to about 2×10⁷ EpCAM proteins, about 4×10⁶ EpCAM proteins to about 6×10⁶ EpCAM proteins, about 4×10⁶ EpCAM proteins to about 7×10⁶ EpCAM proteins, about 4×10⁶ EpCAM proteins to about 15×10⁶ EpCAM proteins, about 4×10⁶ EpCAM proteins to about 2×10⁷ EpCAM proteins, about 6×10⁶ EpCAM proteins to about 7×10⁶ EpCAM proteins. about 6×10⁶ EpCAM proteins to about 15×10⁶ EpCAM proteins, about 6×10⁶ EpCAM proteins to about 2×10⁷ EpCAM proteins, or about 15×10⁶ EpCAM proteins to about 2×10⁷ EpCAM proteins. In some cases, the EpCAM proteins are expressed on the surface of a cell. In some cases, the cell is a tumor cell. In some cases, the cell is derived from a ductal pancreatic adenocarcinoma, an ovarian clear cell adenocarcinoma, a colon adenocarcinoma, a lung adenocarcinoma, an ovarian adeno carcinoma, or a vulvar squamous cell carcinoma. In some cases, the number of EpCAM proteins expressed on a cell is measured by flow cytometry. In some cases, the number of EpCAM proteins expressed on a cell is measured by quantitative flow cytometry.

In some embodiments, a bispecific protein with a CD47-binding domain and an EpCAM-binding domain has enhanced affinity for a cell that expresses CD47 and EpCAM compared to a bivalent protein with one or more CD47-binding domains and/or a bivalent protein with one or more EpCAM-binding domains. In some instances, the bispecific protein has 1.5, 2, 3, 4, 5, or 10-fold higher affinity for the CD47-expressing cell than a bivalent protein that binds to CD47 or a bivalent protein that binds to EpCAM. In some embodiments, a bispecific protein with a CD47-binding domain and an EpCAM-binding domain has enhanced affinity for a cell that expresses higher levels of CD47 than EpCAM compared to a bivalent protein with a CD47-binding domain. In some instances, the bispecific protein has 1.5, 2, 3, 4, 5, or 10-fold higher affinity for the cell with higher EpCAM expression than CD47 expression compared to a bivalent protein that binds to CD47.

In some embodiments, a bispecific antibody with a CD47 binding domain and an EpCAM binding domain has decreased red blood cell (RBC) binding compared to CD47 BMK-1, CD47 BMK-2, or CD47 BMK-4. In some cases, the decreased RBC binding is a decrease of at least 10%, 20%, 30%, 40%, 50%, 60%, 70%, 80%, 90%, 95%, or 100% compared to CD47 BMK-1. In some cases, the decreased RBC binding is a decrease of at least 10%, 20%, 30%, 40%, 50%, 60%, 70%, 80%, 90%, 95%, or 100% compared to CD47 BMK-2. In some cases, the decreased RBC binding is a decrease of at least 10%, 20%, 30%, 40%, 50%, 60%, 70%, 80%, 90%, 95%, or 100% compared to CD47 BMK-4.

In some embodiments, a bispecific antibody of the present disclosure that comprises a first component that binds CD47 and a second component that binds EGFR, binds to a cell that expresses on its surface target antigens of the bispecific protein, with at least 2-50 fold, 10-100 fold, 2-fold, 5-fold, 10-fold, 25-fold, 50-fold or 100-fold, or 20-50%, 50-100%, 20%, 25%, 30%, 40%, 50%, 60%, 70%, 80%, 90% or 100% or more higher affinity (e.g., preferentially binds) compared to the binding affinity of an antibody that is bivalent to only one of CD47 or EGFR, to the cell.

In some embodiments, a bispecific protein provided herein binds to a target cell that expresses a higher level of EGFR than CD47, on its surface. For instance, the ratio of EGFR to CD47 protein expression on the target cell surface, is from about 1, 1.5, 2.0, 2.5, 5, 10, 15, 20, 25, 50, 100, or greater than 200 as measured by flow cytometry.

In some embodiments, a bispecific protein provided herein binds to a target cell that expresses a higher level of CD47 than EGFR, on its surface. For instance, the ratio of CD47 to EGFR protein expression on the target cell surface, is from about 1, 1.5, 2.0, 2.5, 5, 10, 15, 20, 50, 100, or greater than 200 as measured by flow cytometry.

In some embodiments, a bispecific protein provided herein binds to a target cell that expresses equal levels of CD47 and EGFR, on its surface. For instance, the ratio of CD47 to EGFR protein expression on the target cell surface, is about 1 as measured by flow cytometry.

In some cases, a cell expresses about 35,000 CD47 proteins to about 250,000 CD47 proteins. In some cases, a cell expresses at least about 35,000 CD47 proteins. In some cases, a cell expresses at most about 250,000 CD47 proteins. In some cases, a cell expresses about 35,000 CD47 proteins to about 40,000 CD47 proteins, about 35,000 CD47 proteins to about 45,000 CD47 proteins, about 35,000 CD47 proteins to about 60,000 CD47 proteins, about 35,000 CD47 proteins to about 100,000 CD47 proteins, about 35,000 CD47 proteins to about 150,000 CD47 proteins, about 35,000 CD47 proteins to about 250,000 CD47 proteins, about 40,000 CD47 proteins to about 45,000 CD47 proteins, about 40,000 CD47 proteins to about 60,000 CD47 proteins, about 40,000 CD47 proteins to about 120,000 CD47 proteins, about 40,000 CD47 proteins to about 150,000 CD47 proteins, about 40,000 CD47 proteins to about 250,000 CD47 proteins, about 45,000 CD47 proteins to about 60,000 CD47 proteins, about 45,000 CD47 proteins to about 120,000 CD47 proteins, about 45,000 CD47 proteins to about 150,000 CD47 proteins, about 45,000 CD47 proteins to about 250,000 CD47 proteins, about 60,000 CD47 proteins to about 120,000 CD47 proteins, about 60,000 CD47 proteins to about 150,000 CD47 proteins, about 60,000 CD47 proteins to about 250,000 CD47 proteins, about 120,000 CD47 proteins to about 150,000 CD47 proteins, about 120,000 CD47 proteins to about 250,000 CD47 proteins, or about 150,000 CD47 proteins to about 250,000 CD47 proteins. In some cases, the CD47 proteins are expressed on the surface of a cell. In some cases, the cell is a tumor cell. In some cases, the cell is derived from a ductal pancreatic adenocarcinoma, an ovarian clear cell adenocarcinoma, a colon adenocarcinoma, a lung adenocarcinoma, an ovarian adeno carcinoma, or a vulvar squamous cell carcinoma. In some cases, the number of CD47 proteins expressed on a cell is measured by flow cytometry. In some cases, the number of CD47 proteins expressed on a cell is measured by quantitative flow cytometry.

In some cases, a cell expresses about 35,000 EGFR proteins to about 2×10⁷ EGFR proteins. In some cases, a cell expresses at least about 35,000 EGFR proteins. In some cases, a cell expresses at most about 2×10⁷ EGFR proteins. In some cases, a cell expresses about 35,000 EGFR proteins to about 85,000 EGFR proteins, about 35,000 EGFR proteins to about 170,000 EGFR proteins, about 35,000 EGFR proteins to about 300,000 EGFR proteins, about 35,000 EGFR proteins to about 400,000 EGFR proteins, about 35,000 EGFR proteins to about 650,000 EGFR proteins, about 35,000 EGFR proteins to about 750,000 EGFR proteins, about 35,000 EGFR proteins to about 1,500,000 EGFR proteins, about 35,000 EGFR proteins to about 1,900,000 EGFR proteins, about 35,000 EGFR proteins to about 4×10⁶ EGFR proteins, about 35,000 EGFR proteins to about 6×10⁶ EGFR proteins, about 35,000 EGFR proteins to about 7×10⁶ EGFR proteins, about 35,000 EGFR proteins to about 15×10⁶ EGFR proteins, about 35,000 EGFR proteins to about 2×10⁷ EGFR proteins, about 85,000 EGFR proteins to about 170,000 EGFR proteins, about 85,000 EGFR proteins to about 300,000 EGFR proteins, about 85,000 EGFR proteins to about 400,000 EGFR proteins, about 85,000 EGFR proteins to about 650,000 EGFR proteins, about 85,000 EGFR proteins to about 750,000 EGFR proteins, about 85,000 EGFR proteins to about 1,500,000 EGFR proteins, about 85,000 EGFR proteins to about 1,900,000 EGFR proteins, about 85,000 EGFR proteins to about 4×10⁶ EGFR proteins, about 85,000 EGFR proteins to about 6×10⁶ EGFR proteins, about 85,000 EGFR proteins to about 7×10⁶ EGFR proteins, about 85,000 EGFR proteins to about 15×10⁶ EGFR proteins, about 85,000 EGFR proteins to about 2×10⁷ EGFR proteins, about 170,000 EGFR proteins to about 300,000 EGFR proteins, about 170,000 EGFR proteins to about 400,000 EGFR proteins, about 170,000 EGFR proteins to about 650,000 EGFR proteins, about 170,000 EGFR proteins to about 750,000 EGFR proteins, about 170,000 EGFR proteins to about 1,500,000 EGFR proteins, about 170,000 EGFR proteins to about 1,900,000 EGFR proteins, about 170,000 EGFR proteins to about 4×10⁶ EGFR proteins, about 170,000 EGFR proteins to about 6×10⁶ EGFR proteins, about 170,000 EGFR proteins to about 7×10⁶ EGFR proteins, about 170,000 EGFR proteins to about 15×10⁶ EGFR proteins, about 170,000 EGFR proteins to about 2×10⁷ EGFR proteins, about 300,000 EGFR proteins to about 400,000 EGFR proteins, about 300,000 EGFR proteins to about 650,000 EGFR proteins, about 300,000 EGFR proteins to about 750,000 EGFR proteins, about 300,000 EGFR proteins to about 1,500,000 EGFR proteins, about 300,000 EGFR proteins to about 1,900,000 EGFR proteins, about 300,000 EGFR proteins to about 4×10⁶ EGFR proteins, about 300,000 EGFR proteins to about 6×10⁶ EGFR proteins, about 300,000 EGFR proteins to about 7×10⁶ EGFR proteins, about 300,000 EGFR proteins to about 15×10⁶ EGFR proteins, about 300,000 EGFR proteins to about 2×10⁷ EGFR proteins, about 400,000 EGFR proteins to about 650,000 EGFR proteins, about 400,000 EGFR proteins to about 750,000 EGFR proteins, about 400,000 EGFR proteins to about 1,500,000 EGFR proteins, about 400,000 EGFR proteins to about 1,900,000 EGFR proteins, about 400,000 EGFR proteins to about 4×10⁶ EGFR proteins, about 400,000 EGFR proteins to about 6×10⁶ EGFR proteins, about 400,000 EGFR proteins to about 7×10⁶ EGFR proteins, about 400,000 EGFR proteins to about 15×10⁶ EGFR proteins, about 400,000 EGFR proteins to about 2×10⁷ EGFR proteins, about 650,000 EGFR proteins to about 750,000 EGFR proteins, about 650,000 EGFR proteins to about 1,500,000 EGFR proteins, about 650,000 EGFR proteins to about 1,900,000 EGFR proteins, about 650,000 EGFR proteins to about 4×10⁶ EGFR proteins, about 650,000 EGFR proteins to about 6×10⁶ EGFR proteins, about 650,000 EGFR proteins to about 7×10⁶ EGFR proteins, about 650,000 EGFR proteins to about 15×10⁶ EGFR proteins, about 650,000 EGFR proteins to about 2×10⁷ EGFR proteins, about 750,000 EGFR proteins to about 1,500,000 EGFR proteins, about 750,000 EGFR proteins to about 1,900,000 EGFR proteins, about 750,000 EGFR proteins to about 4×10⁶ EGFR proteins, about 750,000 EGFR proteins to about 6×10⁶ EGFR proteins, about 750,000 EGFR proteins to about 7×10⁶ EGFR proteins, about 750,000 EGFR proteins to about 15×10⁶ EGFR proteins, about 750,000 EGFR proteins to about 2×10⁷ EGFR proteins, about 1,500,000 EGFR proteins to about 1,900,000 EGFR proteins, about 1,500,000 EGFR proteins to about 4×10⁶ EGFR proteins, about 1,500,000 EGFR proteins to about 6×10⁶ EGFR proteins, about 1,500,000 EGFR proteins to about 7×10⁶ EGFR proteins, about 1,500,000 EGFR proteins to about 15×10⁶ EGFR proteins, about 1,500,000 EGFR proteins to about 2×10⁷ EGFR proteins, about 1,900,000 EGFR proteins to about 4×10⁶ EGFR proteins, about 1,900,000 EGFR proteins to about 6×10⁶ EGFR proteins, about 1,900,000 EGFR proteins to about 7×10⁶ EGFR proteins, about 1,900,000 EGFR proteins to about 15×10⁶ EGFR proteins, about 1,900,000 EGFR proteins to about 2×10⁷ EGFR proteins, about 4×10⁶ EGFR proteins to about 6×10⁶ EGFR proteins, about 4×10⁶ EGFR proteins to about 7×10⁶ EGFR proteins, about 4×10⁶ EGFR proteins to about 15×10⁶ EGFR proteins, about 4×10⁶ EGFR proteins to about 2×10⁷ EGFR proteins, about 6×10⁶ EGFR proteins to about 7×10⁶ EGFR proteins. about 6×10⁶ EGFR proteins to about 15×10⁶ EGFR proteins, about 6×10⁶ EGFR proteins to about 2×10⁷ EGFR proteins, or about 15×10⁶ EGFR proteins to about 2×10⁷ EGFR proteins. In some cases, the EGFR proteins are expressed on the surface of a cell. In some cases, the cell is a tumor cell. In some cases, the cell is derived from a ductal pancreatic adenocarcinoma, an ovarian clear cell adenocarcinoma, a colon adenocarcinoma, a lung adenocarcinoma, an ovarian adeno carcinoma, or a vulvar squamous cell carcinoma. In some cases, the number of EGFR proteins expressed on a cell is measured by flow cytometry. In some cases, the number of EGFR proteins expressed on a cell is measured by quantitative flow cytometry.

In some embodiments, a bispecific protein with a CD47-binding domain and an EGFR-binding domain has enhanced affinity for a cell that expresses CD47 and EGFR compared to a bivalent protein with one or more CD47-binding domains and/or a bivalent protein with one or more EGFR-binding domains. In some instances, the bispecific protein has 1.5, 2, 3, 4, 5, or 10-fold higher affinity for the CD47-expressing cell than a bivalent protein that binds to CD47 or a bivalent protein that binds to EGFR. In some embodiments, a bispecific protein with a CD47-binding domain and an EGFR-binding domain has enhanced affinity for a cell that expresses higher levels of CD47 than EGFR compared to a bivalent protein with a CD47-binding domain. In some instances, the bispecific protein has 1.5, 2, 3, 4, 5, or 10-fold higher affinity for the cell with higher EGFR expression than CD47 expression compared to a bivalent protein that binds to CD47.

In some embodiments, a bispecific antibody with a CD47 binding domain and an EGFR binding domain has decreased red blood cell (RBC) binding compared to CD47 BMK-1 or CD47 BMK-4. In some cases, the decreased RBC binding is a decrease of at least 10%, 20%, 30%, 40%, 50%, 60%, 70%, 80%, 90%, 95%, or 100% compared to CD47 BMK-1. In some cases, the decreased RBC binding is a decrease of at least 10%, 20%, 30%, 40%, 50%, 60%, 70%, 80%, 90%, 95%, or 100% compared to CD47 BMK-4.

### 4.7 IMMUNOLOGICAL ACTIVITY OF BISPECIFIC ANTIBODIES ON TARGET CELLS

In some embodiments, a bispecific antibody with a CD47-binding domain and an EpCAM-binding domain has a higher immunologic activity against a CD47-expressing cell compared to a bivalent antibody with a CD47-binding domain and/or a bivalent antibody with an EpCAM-binding domain. In some instances, the bispecific antibody has a 1.5, 2, 3, 4, 5, or 10-fold higher immunological activity than a bivalent antibody with a CD47-binding domain and/or a bivalent antibody with an EpCAM-binding domain. Various immunological activities of a bispecific antibody can be measured in in vitro assays such as an ADCC assay and an ADCP assay. In some instances, the bispecific antibody has a 1.5, 2, 3, 4, 5, or 10-fold higher ADCC activity than a bivalent antibody with a CD47-binding domain and/or a bivalent antibody with an EpCAM-binding domain. In some instances, the bispecific antibody has a 1.5, 2, 3, 4, 5, or 10-fold higher ADCP activity than a bivalent antibody with a CD47-binding domain and/or a bivalent antibody with an EpCAM-binding domain.

In some embodiments, a bispecific antibody with a CD47-binding domain and an EpCAM-binding domain has a higher immunologic activity against a CD47-expressing cell compared to a bivalent antibody with a CD47-binding domain and/or a bivalent antibody with an EpCAM-binding domain. In some instances, the bispecific antibody has a 10-20%, 21-30%, 31-40%, 41-50% or at least 51% higher immunological activity than a bivalent antibody with a CD47-binding domain and/or a bivalent antibody with an EpCAM-binding domain. Various immunological activities of a bispecific antibody can be measured in in vitro assays such as an ADCC assay and an ADCP assay. In some instances, the bispecific antibody has a 10-20%, 21-30%, 31-40%, 41-50% or at least 51% higher ADCC activity than a bivalent antibody with a CD47-binding domain and/or a bivalent antibody with an EpCAM-binding domain. In some instances, the bispecific antibody has a 10-20%, 21-30%, 31-40%, 41-50% or at least 51% higher ADCP activity than a bivalent antibody with a CD47-binding domain and/or a bivalent antibody with an EpCAM-binding domain.

In some embodiments, described herein is a bispecific antibody which comprises a first targeting moiety that specifically binds to CD47 and a second targeting moiety that specifically binds to EpCAM. In some instances, the bispecific antibody further comprises an enhanced ADCP effect compared to an ADCP effect by reference antibody CD47 BMK-1. In some instances, the bispecific antibody further comprises an enhanced ADCC effect compared to an ADCC effect by reference antibody CD47 BMK-1. In some cases, the enhanced ADCP is at least 2-fold, 3-fold, 4-fold, or higher than the ADCP effect of reference antibody CD47 BMK-1. In some cases, the enhanced ADCP is at least 30%, 40%, 50%, 60%, 70%, 80%, 90%, or higher than the CD47 BMK-1 effect of reference antibody CD47 BMK-1. In some cases, the enhanced ADCC is at least 2-fold, 3-fold, 4-fold, 5-fold, or higher than the ADCC effect of reference antibody CD47 BMK-1. In some cases, the enhanced ADCC is at least 30%, 40%, 50%, 60%, 70%, 80%, 90%, or higher than the ADCC effect of reference antibody CD47 BMK-1.

In some embodiments, described herein is bispecific antibody comprising a first component that binds specifically to CD47 and a second component that binds specifically to EpCAM, wherein the bispecific antibody mediates ADCC more efficiently than a bivalent antibody that comprises either the first component or the second component, wherein the ADCC activity is determined using an in vitro cytotoxicity assay. In some embodiments, the bispecific antibody mediates at least about 5%, at least about 10%, at least about 20%, at least about 30%, at least about 40%, at least about 50%, at least about 75%, at least about 100% higher maximum cytotoxicity in an in vitro ADCC assay than the bivalent antibody that comprises either the first component or the second component. In some embodiments, the bispecific antibody mediates at least 2-fold, at least 3-fold, at least 4-fold, at least 5-fold or at least 10-fold higher maximum cytotoxicity in an in vitro ADCC assay than the bivalent antibody that comprises either the first component or the second component.

In some embodiments, described herein is bispecific antibody comprising a first component that binds specifically to CD47 and a second component that binds specifically to EpCAM, wherein the bispecific antibody mediates antibody dependent cellular phagocytosis (ADCP) more efficiently than a bivalent antibody that comprises either the first component or the second component, wherein the ADCP activity is determined using an in vitro FACS based phagocytosis assay. In some embodiments, the bispecific antibody mediates at least about 5%, at least about 10%, at least about 20%, at least about 30%, at least about 40%, at least about 50%, at least about 75%, at least about 100% higher maximum phagocytosis in an in vitro ADCP assay than the bivalent antibody that comprises either the first component or the second component.

In some instances, the bispecific antibody further comprises reduced cell killing by immune cells compared to the killing by immune cells of the reference antibody CD47 BMK-1. In some cases, the cell killing is improved by about 10%, 20%, 30%, 40%, 50%, 60%, 70%, 80%, 90%, or higher compared to the immune cell viability in the presence of reference antibody CD47 BMK-1. In some cases, the immune cell is a Natural Killer cell. In some cases, the immune cell is a phagocytic cell. In some cases, the immune cell is a macrophage.

Immunological activity can also be measured in a cell-line derived xenograft assay, wherein transformed cells are injected into mice and form a tumor. In some instances, a bispecific protein with a CD47-binding domain and an EpCAM-binding domain inhibits the growth of a tumor comprising CD47-expressing cells to a greater extent than a bivalent protein with the same CD47-binding domain and/or a bivalent protein with the same EpCAM-binding domain. In some instances, the bispecific protein exhibits 1.5, 2, 3, 4, 5, or 10-fold higher inhibition of xenograft tumor growth compared to a bivalent protein with a CD47-binding domain and/or a bivalent protein with an EpCAM-binding domain.

In some embodiments, a bispecific antibody with a CD47-binding domain and an EGFR-binding domain has a higher immunologic activity against a CD47-expressing cell compared to a bivalent antibody with a CD47-binding domain and/or a bivalent antibody with an EGFR-binding domain. In some instances, the bispecific antibody has a 1.5, 2, 3, 4, 5, or 10-fold higher immunological activity than a bivalent antibody with a CD47-binding domain and/or a bivalent antibody with an EGFR-binding domain. Various immunological activities of a bispecific antibody can be measured in in vitro assays such as an ADCC assay and an ADCP assay. In some instances, the bispecific antibody has a 1.5, 2, 3, 4, 5, or 10-fold higher ADCC activity than a bivalent antibody with a CD47-binding domain and/or a bivalent antibody with an EGFR-binding domain. In some instances, the bispecific antibody has a 1.5, 2, 3, 4, 5, or 10-fold higher ADCP activity than a bivalent antibody with a CD47-binding domain and/or a bivalent antibody with an EGFR-binding domain.

In some embodiments, a bispecific antibody with a CD47-binding domain and an EGFR-binding domain has a higher immunologic activity against a CD47-expressing cell compared to a bivalent antibody with a CD47-binding domain and/or a bivalent antibody with an EGFR-binding domain. In some instances, the bispecific antibody has a 10-20%, 21-30%, 31-40%, 41-50% or at least 51% higher immunological activity than a bivalent antibody with a CD47-binding domain and/or a bivalent antibody with an EGFR-binding domain. Various immunological activities of a bispecific antibody can be measured in in vitro assays such as an ADCC assay and an ADCP assay. In some instances, the bispecific antibody has a 10-20%, 21-30%, 31-40%, 41-50% or at least 51% higher ADCC activity than a bivalent antibody with a CD47-binding domain and/or a bivalent antibody with an EGFR-binding domain. In some instances, the bispecific antibody has a 10-20%, 21-30%, 31-40%, 41-50% or at least 51% higher ADCP activity than a bivalent antibody with a CD47-binding domain and/or a bivalent antibody with an EGFR-binding domain.

In some embodiments, described herein is a bispecific antibody which comprises a first targeting moiety that specifically binds to CD47 and a second targeting moiety that specifically binds to EGFR. In some instances, the bispecific antibody further comprises an enhanced ADCP effect compared to an ADCP effect by reference antibody CD47 BMK-1. In some instances, the bispecific antibody further comprises an enhanced ADCC effect compared to an ADCC effect by reference antibody CD47 BMK-1. In some cases, the enhanced ADCP is at least 2-fold, 3-fold, 4-fold, or higher than the ADCP effect of reference antibody CD47 BMK-1. In some cases, the enhanced ADCP is at least 30%, 40%, 50%, 60%, 70%, 80%, 90%, or higher than the CD47 BMK-1 effect of reference antibody CD47 BMK-1. In some cases, the enhanced ADCC is at least 2-fold, 3-fold, 4-fold, 5-fold, or higher than the ADCC effect of reference antibody CD47 BMK-1. In some cases, the enhanced ADCC is at least 30%, 40%, 50%, 60%, 70%, 80%, 90%, or higher than the ADCC effect of reference antibody CD47 BMK-1.

In some embodiments, described herein is bispecific antibody comprising a first component that binds specifically to CD47 and a second component that binds specifically to EGFR, wherein the bispecific antibody mediates ADCC more efficiently than a bivalent antibody that comprises either the first component or the second component, wherein the ADCC activity is determined using an in vitro cytotoxicity assay. In some embodiments, the bispecific antibody mediates at least about 5%, at least about 10%, at least about 20%, at least about 30%, at least about 40%, at least about 50%, at least about 75%, at least about 100% higher maximum cytotoxicity in an in vitro ADCC assay than the bivalent antibody that comprises either the first component or the second component. In some embodiments, the bispecific antibody mediates at least 2-fold, at least 3-fold, at least 4-fold, at least 5-fold or at least 10-fold higher maximum cytotoxicity in an in vitro ADCC assay than the bivalent antibody that comprises either the first component or the second component.

In some embodiments, described herein is bispecific antibody comprising a first component that binds specifically to CD47 and a second component that binds specifically to EGFR, wherein the bispecific antibody mediates antibody dependent cellular phagocytosis (ADCP) more efficiently than a bivalent antibody that comprises either the first component or the second component, wherein the ADCP activity is determined using an in vitro FACS based phagocytosis assay. In some embodiments, the bispecific antibody mediates at least about 5%, at least about 10%, at least about 20%, at least about 30%, at least about 40%, at least about 50%, at least about 75%, at least about 100% higher maximum phagocytosis in an in vitro ADCP assay than the bivalent antibody that comprises either the first component or the second component.

In some instances, the bispecific antibody further comprises reduced cell killing by immune cells compared to the killing by immune cells of the reference antibody CD47 BMK-1. In some cases, the cell killing is improved by about 10%, 20%, 30%, 40%, 50%, 60%, 70%, 80%, 90%, or higher compared to the immune cell viability in the presence of reference antibody CD47 BMK-1. In some cases, the immune cell is a Natural Killer cell. In some cases, the immune cell is a phagocytic cell. In some cases, the immune cell is a macrophage.

Immunological activity can also be measured in a cell-line derived xenograft assay, wherein transformed cells are injected into mice and form a tumor. In some instances, a bispecific protein with a CD47-binding domain and an EGFR-binding domain inhibits the growth of a tumor comprising CD47-expressing cells to a greater extent than a bivalent protein with the same CD47-binding domain and/or a bivalent protein with the same EGFR-binding domain. In some instances, the bispecific protein exhibits 1.5, 2, 3, 4, 5, or 10-fold higher inhibition of xenograft tumor growth compared to a bivalent protein with a CD47-binding domain and/or a bivalent protein with an EGFR-binding domain.

As used herein, “Antibody-dependent cell-mediated cytotoxicity” and “ADCC” refers to a cell-mediated reaction in which non-specific cytotoxic cells (e.g., Natural Killer (NK) cells, neutrophils, and macrophages) recognize bound antibody on a target cell and subsequently cause lysis of the target cell. In one embodiment, the target cell is a human cell, such as a tumor cell (e.g., a myeloma cell). In some embodiments, the tumor cell is a derived from an adenocarcinoma, a lymphoma, a carcinoma. In some cases, the adenocarcinoma is a ductal pancreatic adenocarcinoma, an ovarian clear cell adenocarcinoma, a colon adenocarcinoma, a lung adenocarcinoma, or an ovarian adenocarcinoma. In other cases, the carcinoma is a vulvar squamous cell carcinoma. While not wishing to be bound by any particular mechanism of action, the cytotoxic cells that mediate ADCC generally express Fc receptors (FcRs). Cells for mediating ADCC, NK cells, express FcγRIII, whereas monocytes express FcγRI, FcγRII, FcγRIII and/or FcγRIV. FcR expression on hematopoietic cells is summarized in Ravetch and Kinet, Annu. Rev. Immunol., 9:457-92 (1991).

To assess ADCC activity of a bispecific protein as described herein, an in vitro ADCC assay, such as a cytotoxic assay using cancer cell lines, is carried out in some embodiments. Useful effector cells for such assays include, but are not limited to, peripheral blood mononuclear cells (PBMC) and Natural Killer (NK) cells. Alternatively, or additionally, ADCC activity of the bispecific proteins of interest is assessed, in some embodiments, in vivo, e.g., in an animal.

“Antibody dependent cellular phagocytosis” or “ADCP” refers to the ability of a phagocytic cell (e.g. macrophage) to eliminate an antibody-coated target cell. In some embodiments, a phagocytosis assay is used to measure the ADCP effect.

In some embodiments, a bispecific protein as described herein, that binds CD47 and EpCAM mediates antibody-dependent cellular phagocytosis of at least 50% of the cells in an exponentially growing population of CD47+/EpCAM+ cancer cell. In some embodiments, the tumor cell is a derived from an adenocarcinoma, a lymphoma, a carcinoma. In some cases, the adenocarcinoma is a ductal pancreatic adenocarcinoma, an ovarian clear cell adenocarcinoma, a colon adenocarcinoma, a lung adenocarcinoma, or an ovarian adenocarcinoma.

In some embodiments, a bispecific protein as described herein binds CD47 and EpCAM mediates inhibition of a CD47-SIRPα interaction. In some embodiments the bispecific antibody inhibits binding of SIRPα to CD47 by at least 50%. In some embodiments, binding inhibition is measured by ELISA. In some embodiments binding inhibition is measured with CD47+ cells. In some embodiments, the CD47+ cells are CD47+ EpCAM+ tumor cells.

In some embodiments, the bispecific antibody has reduced binding to CD47+ non-tumor cells such as red blood cells and platelets. In some cases, binding to CD47+ non-tumor cells is reduced by 10%, 20%, 30%, 40%, 50%, 60%, 70%, 80%, 90%, 100% compared to the referee antibody, CD47 BMK-1. In some cases, the bispecific antibody binding to CD47+ non-tumor cells is measured by a red blood cells (RBCs) binding assay or a flow cytometry assay.

In some embodiments, the concentration of bispecific antibody required to induce RBC lysis is at least 2-fold, 3-fold, 4-fold, or higher than the hemolysis effect of reference antibody CD47 BMK-1.

### 4.8 PRODUCTION OF ANTIBODIES OR BINDING FRAGMENTS THEREOF

In some embodiments, polypeptides described herein (e.g., antibodies and its binding fragments) are produced using any method known in the art to be useful for the synthesis of polypeptides (e.g., antibodies), in particular, by chemical synthesis or by recombinant expression, and are preferably produced by recombinant expression techniques.

In some instances, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some instances, an antibody or its binding is optionally generated by immunizing an animal, such as a mouse, to generate polyclonal antibodies or, more preferably, by generating monoclonal antibodies, e.g., as described by Kohler and Milstein (1975, Nature 256:495-497) or, as described by Kozbor et al. (1983, Immunology Today 4:72) or Cole et al. (1985 in Monoclonal Antibodies and Cancer Therapy, Alan R. Liss, Inc., pp. 77-96). Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

In some embodiments, techniques developed for the production of “chimeric antibodies” (Morrison et al., 1984, Proc. Natl. Acad. Sci. 81:851-855; Neuberger et al., 1984, Nature 312:604-608; Takeda et al., 1985, Nature 314:452-454) by splicing genes from a mouse antibody molecule of appropriate antigen specificity together with genes from a human antibody molecule of appropriate biological activity are used. A chimeric antibody is a molecule in which different portions are derived from different animal species, such as those having a variable region derived from a murine monoclonal antibody and a human immunoglobulin constant region.

In some embodiments, techniques described for the production of single chain antibodies (U.S. Pat. No. 4,694,778; Bird, 1988, Science 242:423-42; Huston et al., 1988, Proc. Natl. Acad. Sci. USA 85:5879-5883; and Ward et al., 1989, Nature 334:544-54) are adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli are also optionally used (Skerra et al., 1988, Science 242:1038-1041).

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host-expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some instances, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some instances, a number of selection systems are used, including but not limited to the herpes simplex virus thymidine kinase (Wigler et al., 1977, Cell 11:223), hypoxanthine-guanine phosphoribosyltransferase (Szybalska & Szybalski, 192, Proc. Natl. Acad. Sci. USA 48:202), and adenine phosphoribosyltransferase (Lowy et al., 1980, Cell 22:817) genes are employed in tk-, hgprt- or aprt-cells, respectively. Also, antimetabolite resistance are used as the basis of selection for the following genes: dhfr, which confers resistance to methotrexate (Wigler et al., 1980, Proc. Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May 1993, TIB TECH 11(5):155-215) and hygro, which confers resistance to hygromycin (Santerre et al., 1984, Gene 30:147). Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some instances, the expression levels of an antibody are increased by vector amplification (for a review, see Bebbington and Hentschel, the use of vectors based on gene amplification for the expression of cloned genes in mammalian cells in DNA cloning, Vol. 3. (Academic Press, New York, 1987)). When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase (Crouse et al., 1983, Mol. Cell Biol. 3:257).

In some instances, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### 4.9 EXPRESSION VECTORS

In some embodiments, vectors include any suitable vectors derived from either a eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichia pastoris vector, pGAPZA, B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include pRK5, p3×FLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3×FLAG-CMV 7.1, pFLAG-CMV 20, p3×FLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3×FLAG-CMV 9, p3×FLAG-CMV 13, pFLAG-Myc-CMV 21, p3×FLAG-Myc-CMV 25, pFLAG-CMV 4, p3×FLAG-CMV 10, p3×FLAG-CMV 14, pFLAG-Myc-CMV 22, p3×FLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### 4.10 HOST CELLS

In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria or Thermotogae. A bacterial cell can be Escherichia coli, Clostridium botulinum, or Coli bacilli.

Exemplary prokaryotic host cells include, but are not limited to, BL21, Mach1™, DH10B™, TOP10, DH5α, DH10Bac™, OmniMax™, MegaX™, DH12S™, INV110, TOP10F′, INVαF, TOP10/P3, ccdB Survival, PIR1, PIR2, Stb12™, Stb13™, or Stb14™.

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVSc1.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293 H cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High Five™ cells, and expresSF+® cells.

In some instances, plant cells include a cell from algae. Exemplary insect cell lines include, but are not limited to, strains from Chlamydomonas reinhardtii 137c, or Synechococcus elongatus PPC 7942.

### 4.11 CERTAIN TERMINOLOGY

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

“Antibodies” and “immunoglobulins” (Igs) are glycoproteins having the same structural characteristics. The terms are used synonymously. In some instances, the antigen specificity of the immunoglobulin is known.

The term “antibody” is used in the broadest sense and covers fully assembled antibodies, antibody fragments that can bind antigen (e.g., Fab, F(ab′)2, Fv, single chain antibodies, diabodies, antibody chimeras, hybrid antibodies, bispecific antibodies, and the like), and recombinant peptides comprising the forgoing.

The terms “monoclonal antibody” and “mAb” as used herein refer to an antibody obtained from a substantially homogeneous population of antibodies, i.e., the individual antibodies comprising the population are identical except for possible naturally occurring mutations that may be present in minor amounts.

The term “bispecific antibody” is used to refer to an antibody that binds specifically to at least two different antigens, and includes antibodies that can only bind specifically to two different antigens, and also includes antibodies that can bind specifically to two different antigens and further includes or is conjugated to one or more additional binding domains that bind specifically to a third, a fourth, or more antigens.

“Native antibodies” and “native immunoglobulins” are usually heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy-chain variable domains.

The term “variable” refers to the fact that certain portions of the variable domains differ extensively in sequence among antibodies. Variable regions confer antigen-binding specificity. However, the variability is not evenly distributed throughout the variable domains of antibodies. It is concentrated in three segments called complementarity determining regions (CDRs) or hypervariable regions, both in the light chain and the heavy-chain variable domains. The more highly conserved portions of variable domains are celled in the framework (FR) regions. The variable domains of native heavy and light chains each comprise four FR regions, largely adopting a β-pleated-sheet configuration, connected by three CDRs, which form loops connecting, and in some cases forming part of, the β-pleated-sheet structure. The CDRs in each chain are held together in close proximity by the FR regions and, with the CDRs from the other chain, contribute to the formation of the antigen-binding site of antibodies (see, Kabat et al. (1991) NIH PubL. No. 91-3242, Vol. I, pages 647-669). The constant domains are not involved directly in binding an antibody to an antigen, but exhibit various effector functions, such as Fc receptor (FcR) binding, participation of the antibody in antibody-dependent cellular cytotoxicity, initiation of complement dependent cytotoxicity, and mast cell degranulation.

The term “hypervariable region,” when used herein, refers to the amino acid residues of an antibody that are responsible for antigen-binding. The hypervariable region comprises amino acid residues from a “complementarily determining region” or “CDR” (i.e., residues 24-34 (L1), 50-56 (L2), and 89-97 (L3) in the light-chain variable domain and 31-35 (H1), 50-65 (H2), and 95-102 (H3) in the heavy-chain variable domain; Kabat et al. (1991) Sequences of Proteins of Immunological Interest, 5th Ed. Public Health Service, National Institute of Health, Bethesda, Md.) and/or those residues from a “hypervariable loop” (i.e., residues 26-32 (L1), 50-52 (L2), and 91-96 (L3) in the light-chain variable domain and (H1), 53-55 (H2), and 96-101 (13) in the heavy chain variable domain; Clothia and Lesk, (1987) J. Mol. Biol., 196:901-917). “Framework” or “FR” residues are those variable domain residues other than the hypervariable region residues, as herein deemed.

“Antibody fragments” comprise a portion of an intact antibody, preferably the antigen-binding or variable region of the intact antibody. Examples of antibody fragments include Fab, Fab, F(ab′)2, and Fv fragments; diabodies; linear antibodies (Zapata et al. (1995) Protein Eng. 10:1057-1062); single-chain antibody molecules; and bispecific antibodies formed from antibody fragments. Papain digestion of antibodies produces two identical antigen-binding fragments, called “Fab” fragments, each with a single antigen-binding site, and a residual “Fc” fragment, whose name reflects its ability to crystallize readily. Pepsin treatment yields an F(ab′)2 fragment that has two antigen-combining sites and is still capable of cross-linking antigen.

“Fv” is the minimum antibody fragment that contains a complete antigen recognition and binding site. This region consists of a dimer of one heavy- and one light-chain variable domain in tight, non-covalent association. It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment also contains the constant domain of the light chain and the first constant domain (CH1) of the heavy chain. Fab fragments differ from Fab′ fragments by the addition of a few residues at the carboxy terminus of the heavy chain CH1 domain including one or more cysteines from the antibody hinge region. Fab′-SH is the designation herein for Fab′ in which the cysteine residue(s) of the constant domains bear a free thiol group. Fab′ fragments are produced by reducing the F(ab′)2 fragment's heavy chain disulfide bridge. Other chemical couplings of antibody fragments are also known.

The “light chains” of antibodies (immunoglobulins) from any vertebrate species can be assigned to one of two clearly distinct types, called kappa (κ) and lambda (λ), based on the amino acid sequences of their constant domains.

Depending on the amino acid sequence of the constant domain of their heavy chains, immunoglobulins can be assigned to different classes. There are five major classes of human immunoglobulins: IgA, IgD, IgE, IgG, IgM, and IgY, and several of these may be further divided into subclasses (isotypes), e.g., IgG1, IgG2, IgG3, IgG4, IgA1, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody dependent cell-mediated cytotoxicity) activity.

In some instances, the CDRs of an antibody is determined according to (i) the Kabat numbering system (Kabat et al. (197) Ann. NY Acad. Sci. 190:382-391 and, Kabat et al. (1991) Sequences of Proteins of Immunological Interest Fifth Edition, U.S. Department of Health and Human Services, NIH Publication No. 91-3242); or (ii) the Chothia numbering scheme, which will be referred to herein as the “Chothia CDRs” (see, e.g., Chothia and Lesk, 1987, J. Mol. Biol., 196:901-917; A1-Lazikani et al., 1997, J. Mol. Biol., 273:927-948; Chothia et al., 1992, J. Mol. Biol., 227:799-817; Tramontano A et al., 1990, J. Mol. Biol. 215(1): 175-82; and U.S. Pat. No. 7,709,226); or (iii) the ImMunoGeneTics (IMGT) numbering system, for example, as described in Lefranc, M.-P., 1999, The Immunologist, 7: 132-136 and Lefranc, M.-P. et al, 1999, Nucleic Acids Res., 27:209-212 (“IMGT CDRs”); or (iv) MacCallum et al, 1996, J. Mol. Biol., 262:732-745. See also, e.g., Martin, A., “Protein Sequence and Structure Analysis of Antibody Variable Domains,” in Antibody Engineering, Kontermann and Diibel, eds., Chapter 31, pp. 422-439, Springer-Verlag, Berlin (2001).

With respect to the Kabat numbering system, CDRs within an antibody heavy chain molecule are typically present at amino acid positions 31 to 35, which optionally can include one or two additional amino acids, following 35 (referred to in the Kabat numbering scheme as 35 A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). As is well known to those of skill in the art, using the Kabat numbering system, the actual linear amino acid sequence of the antibody variable domain can contain fewer or additional amino acids due to a shortening or lengthening of a FR and/or CDR and, as such, an amino acid's Kabat number is not necessarily the same as its linear amino acid number.

The term “chimeric” antibody refers to an antibody in which a portion of the heavy and/or light chain is derived from a particular source or species, while the remainder of the heavy and/or light chain is derived from a different source or species.

The term “human antibody” or “humanized antibody”, as used herein, is intended to include antibodies having variable and constant regions derived from human germ line immunoglobulin sequences. Human antibodies are well-known in the state of the art (van Dijk, M. A., and van de Winkel, J. G., Curr. Opin. Chem. Biol. 5 (2001) 368-374). In some instances, human antibodies are also produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al, Proc. Natl. Acad. Sci. USA 90 (1993) 2551-2555; Jakobovits, A., et al, Nature 362 (1993) 255-258; Bruggemann, M., et al, Year Immunol. 7 (1993) 33-40). In additional instances, human antibodies are also produced in phage display libraries (Hoogenboom, H. R., and Winter, G., J. Mol. Biol. 227 (1992) 381-388; Marks, J. D., et al, J. Mol. Biol. 222 (1991) 581-597). The techniques of Cole et al. and Boerner et al. are also available for the preparation of human monoclonal antibodies (Cole, et al., Monoclonal Antibodies and Cancer Therapy, Alan R. Liss, p. 77 (1985); and Boerner, P., et al, J. Immunol. 147 (1991) 86-95).

The term “recombinant human antibody”, as used herein, is intended to include all human antibodies that are prepared, expressed, created or isolated by recombinant means, such as antibodies isolated from a host cell such as a NSO or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. In some cases, the recombinant human antibodies have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the term “Percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

The following list of embodiments of the invention are to be considered as disclosing various features of the invention, which features can be considered to be specific to the particular embodiment under which they are discussed, or which are combinable with the various other features as listed in other embodiments. Thus, simply because a feature is discussed under one particular embodiment does not necessarily limit the use of that feature to that embodiment.

Embodiment A1. A bispecific antibody comprising a CD47-binding domain and an EpCAM binding domain.

Embodiment A2. The bispecific antibody of embodiment A1, further comprising an Fc domain.

Embodiment A3. The bispecific antibody of embodiment A1 or A2, wherein the bispecific antibody has a higher affinity for CD47 expressed on the surface of a tumor cell than for CD47 expressed on the surface of a red blood cell or a platelet.

Embodiment A4. The bispecific antibody of embodiment A3, wherein the tumor cell expresses EpCAM.

Embodiment A5. The bispecific antibody of any of embodiments A1 to A4, wherein a concentration of the bispecific antibody required for half-maximal binding to a human red blood cell is greater than 500 nM.

Embodiment A6. The bispecific antibody of any one of embodiments A1 to A5, wherein bispecific antibody binds to human CD47 with a KD of less than 100 nM.

Embodiment A7. The bispecific antibody of embodiment A6, wherein the bispecific antibody binds to human CD47 with a KD of between 1 nM and 100 nM, between 1 nM and 50 nM, or between 5 nM and 50 nM.

Embodiment A8. The bispecific antibody of any of embodiments A1 to A7, wherein the bispecific antibody binds to EpCAM with a KD of less than 500 nM.

Embodiment A9. The bispecific antibody of embodiment A8, wherein the bispecific antibody binds to EpCAM with a KD of between 0.2 nM and 500 nM, between 1 nM and 300 nM, between 5 nM and 200 nM, or between 10 nM and 150 nM.

Embodiment A10. The bispecific antibody of any of embodiments A6 to A9, wherein the KD is determined by surface plasmon resonance.

Embodiment A11. The bispecific antibody of any of embodiments A1 to A10, wherein the CD47-binding domain is a human or engineered human CD47-binding domain.

Embodiment A12. The bispecific antibody of any of embodiments A1 to A11, wherein the CD47-binding domain comprises a heavy chain variable domain and a light chain variable domain.

Embodiment A13. The bispecific antibody of any of embodiments A1 to A11, wherein the CD47-binding domain comprises an scFv.

Embodiment A14. The bispecific antibody of any of embodiments A1 to A13, wherein the EpCAM-binding domain comprises a heavy chain variable domain and a light chain variable domain.

Embodiment A15. The bispecific antibody of any of embodiments A1 to A13, wherein the EpCAM-binding domain comprises an scFv.

Embodiment A16. The bispecific antibody of any of embodiments A1 to A15, wherein the Fc domain is a human Fc domain.

Embodiment A17. The bispecific antibody of embodiment A16, wherein the isotype of the human Fc domain is IgG1 or IgG4.

Embodiment A18. The bispecific antibody of any of embodiments A1 to A17, wherein the Fc domain is a heterodimeric Fc domain, wherein the heterodimeric Fc region comprises a knob chain and a hole chain, forming a knob-into-hole (KiH) structure.

Embodiment A19. The bispecific antibody of embodiment A18, wherein the knob chain comprises the mutation T366W and the hole chain comprises the mutations T366S, L368A, and Y407V, wherein amino acid position numbering is according to the EU index of Kabat et al.

Embodiment A20. The bispecific antibody of any of embodiments A1 to A19, wherein the bispecific antibody has an asymmetric three-chain knob-into-hole structure.

Embodiment A21. The bispecific antibody of embodiment A20, wherein the CD47-binding domain is an scFv.

Embodiment A22. The bispecific antibody of embodiment A20, wherein the EpCAM-binding domain is an scFv.

Embodiment A23. The bispecific antibody of any of embodiments A1 to A22, wherein the CD47-binding domain comprises three heavy chain (HC) complementarity determining regions (CDRs): HC-CDR1, HC-CDR2, and HC-CDR3; and three light chain (LC) CDRs: LC-CDR1, the LC-CDR2, and the LC-CDR3; wherein HC-CDR1 comprises SEQ ID NO: 15, HC-CDR2 comprises SEQ ID NO: 16, HC-CDR3 comprises a sequence with at least 50% identity to SEQ ID NO: 17, LC-CDR1 comprises SEQ ID NO: 18, LC-CDR2 comprises SEQ ID NO: 19, and LC-CDR3 comprises a sequence with at least 50% identity to SEQ ID NO: 20.

Embodiment A24. The bispecific antibody of embodiment A23, wherein HC-CDR3 comprises amino acid substitutions at one or more of K94, E95, G96, S97, F98, G99, V100b, D101, and P102; and LC-CDR3 comprises amino acid substitutions at one or more of Y89, S90, T91, D92, 193, S94, G95, N95a, H95b, W96, and V97.

Embodiment A25. The bispecific antibody of any of embodiments A1 to A24, wherein the heavy chain variable domain of the CD47-binding domain comprises a sequence with at least 90% identity to SEQ ID NO: 5, SEQ ID NO: 10, or SEQ ID NO: 13 and the light chain of the CD47-binding domain comprises a sequence with at least 90% identity to SEQ ID NO: 8, SEQ ID NO: 11, or SEQ ID NO: 14.

Embodiment A26. The bispecific antibody of any of embodiments A1 to A25, wherein the EpCAM-binding domain comprises three heavy chain (HC) complementarity determining regions (CDRs), HC-CDR1, HC-CDR2, and HC-CDR3 and three light chain (LC) CDRs, LC-CDR1, the LC-CDR2, and the LC-CDR3; wherein the CDRs are defined by an ordered set of sequences listed as HC-CDR1, HC-CDR2, HC-CDR3, LC-CDR1, LC-CDR2, LC-CDR3; and wherein the CDRs are selected from the group consisting of sequences having at least 90% identity to:

SEQ ID NO: 45, SEQ ID NO: 46, SEQ ID NO: 47, SEQ ID NO: 81, SEQ ID NO: 82, and SEQ ID NO: 83;

SEQ ID NO: 48, SEQ ID NO: 49, SEQ ID NO: 50, SEQ ID NO: 84, SEQ ID NO: 85, and SEQ ID NO: 86;

SEQ ID NO: 51, SEQ ID NO: 52, SEQ ID NO: 53, SEQ ID NO: 87, SEQ ID NO: 88, and SEQ ID NO: 89;

SEQ ID NO: 54, SEQ ID NO: 55, SEQ ID NO: 56, SEQ ID NO: 90, SEQ ID NO: 91, and SEQ ID NO: 92;

SEQ ID NO: 57, SEQ ID NO: 58, SEQ ID NO: 59, SEQ ID NO: 93, SEQ ID NO: 94, and SEQ ID NO: 95;

SEQ ID NO: 60, SEQ ID NO: 61, SEQ ID NO: 62, SEQ ID NO: 96, SEQ ID NO: 97, and SEQ ID NO: 98;

SEQ ID NO: 63, SEQ ID NO: 64, SEQ ID NO: 65, SEQ ID NO: 99, SEQ ID NO: 100, and SEQ ID NO: 101;

SEQ ID NO: 66, SEQ ID NO: 67, SEQ ID NO: 68, SEQ ID NO: 102, SEQ ID NO: 103, and SEQ ID NO: 104;

SEQ ID NO: 69, SEQ ID NO: 70, SEQ ID NO: 71, SEQ ID NO: 105, SEQ ID NO: 106, and SEQ ID NO: 107;

SEQ ID NO: 72, SEQ ID NO: 72, SEQ ID NO: 74, SEQ ID NO: 108, SEQ ID NO: 109, and SEQ ID NO: 110;

SEQ ID NO: 75, SEQ ID NO: 76, SEQ ID NO: 77, SEQ ID NO: 111, SEQ ID NO: 112, and SEQ ID NO: 113; and

SEQ ID NO: 78, SEQ ID NO: 79, SEQ ID NO: 80, SEQ ID NO: 114, SEQ ID NO: 115, and SEQ ID NO: 116.

Embodiment A27. The bispecific antibody of any of embodiments A1 to A26, wherein the EpCAM-binding domain comprises:

a sequence at least 90% identical to SEQ ID NO: 21 and

a sequence at least 90% identical to SEQ ID NO: 33;

a sequence at least 90% identical to SEQ ID NO: 22 and

a sequence at least 90% identical to SEQ ID NO: 34; or

a sequence at least 90% identical to SEQ ID NO: 24 and

a sequence at least 90% identical to SEQ ID NO: 36.

Embodiment A28. The bispecific antibody of any of embodiments A1 to A27, wherein less than 1 nM or less than 0.01 nM of the bispecific antibody increases a percentage of A431 cells engulfed by macrophage by at least 4-fold compared to a nonspecific IgG1 antibody control.

Embodiment A29. The bispecific antibody of embodiment A28, wherein a concentration of the antibody required to mediate antibody-dependent cellular phagocytosis of an EpCAM-positive, CD47-positive tumor cell by a macrophage is between 0.01 nM and −3 nM.

Embodiment A30. The bispecific antibody of embodiment A29, wherein the EpCAM-positive, CD47-positive tumor cell is an OVISE cell or an A431 cell.

Embodiment A31. The bispecific antibody of embodiment A29, wherein the EpCAM-positive, CD47-positive tumor cell is selected from a ductal pancreatic adenocarcinoma cell, an ovarian clear cell adenocarcinoma cell, a colon adenocarcinoma cell, a lung adenocarcinoma cell, an ovarian adenocarcinoma cell, a vulvar squamous cell carcinoma cell.

Embodiment A32. The bispecific antibody of any of embodiments A1 to A28, wherein 100 nM of the bispecific antibody inhibits the binding of SIRPα to CD47 on the surface of a cell by at least 30%.

Embodiment A33. The bispecific antibody of embodiment A32, wherein the cell is a CD47+ EpCAM+ tumor cell.

Embodiment A34. The bispecific antibody of embodiment A33, wherein the cell expresses at least as many EpCAM proteins on its surface as an HCC-44 cell.

Embodiment A35. The bispecific antibody of embodiment A33, wherein the cell expresses at least 100,000, at least 300,000, at least 600,000 or at least 1,500,000 EpCAM proteins on its surface.

Embodiment A36. The bispecific antibody of any of embodiments A1 to A33, wherein 400 nM of the bispecific antibody does not induce hemolysis of red blood cells in a hemagglutination assay.

Embodiment A37. A complex comprising the bispecific antibody of any of embodiments A1 to A36 and a CD47+ EpCAM+ target cell.

Embodiment A38. The complex of embodiment A37, wherein the target cell expresses at least as many EpCAM proteins on its surface as an HCC-44 cell.

Embodiment A39. The complex of embodiment A37, wherein the target cell expresses at least 100,000, at least 300,000, at least 600,000 or at least 1,500,000 EpCAM proteins on its surface.

Embodiment A40. The complex of any of embodiments A37 to A39, wherein the CD47+ EpCAM+ target cell is a cancer cell.

Embodiment A41. The complex of embodiment A40, wherein the cancer cell is a ductal pancreatic adenocarcinoma cell, an ovarian clear cell adenocarcinoma cell, a colon adenocarcinoma cell, a lung adenocarcinoma cell, an ovarian adenocarcinoma cell, or a vulvar squamous cell carcinoma cell.

Embodiment A42. A method of inducing phagocytosis of a CD47+ EpCAM+ target cell comprising administering the bispecific antibody of any of embodiments A1 to A36.

Embodiment A43. The method of embodiment A42, wherein the bispecific antibody is administered at a concentration of less than 5 nM, less than 2 nM, less than 1 nM, less than 0.5 nM, less than 0.2 nM, less than 0.1 nM, or less than 0.05 nM.

Embodiment A44. A method of killing a CD47+ EpCAM+ target cell comprising administering the bispecific antibody of any of embodiments A1 to A36, wherein the bispecific antibody inhibits the binding of SIRPα to CD47 on the surface of the CD47+ EpCAM+ target cell.

Embodiment A45. A method of killing a CD47+ EpCAM+ target cell comprising administering the bispecific antibody of any of embodiments A1 to A36, wherein the bispecific antibody induces antibody-dependent cellular cytotoxicity that kills the CD47+ EpCAM+ target cell.

Embodiment A46. The method of embodiment A45, wherein the antibody is administered at a concentration of between 0.01-1 nM, between 0.01-0.5 nM, between 0.01-0.25 nM, between 0.01-0.1 nM, between 0.01-0.05 nM, or less than 0.01 nM.

Embodiment A47. The method of embodiment A45 or A46, wherein the CD47+ EpCAM+ target cell is selected from an A431 cell, an HCC-44 cell, a SKOV-3 cell, an OVISE cell, or a CFPAC-1 cell.

Embodiment A48. The method of embodiment A45 or A46, wherein the CD47+ EpCAM+ target cell is selected from a ductal pancreatic adenocarcinoma cell, an ovarian clear cell adenocarcinoma cell, a colon adenocarcinoma cell, a lung adenocarcinoma cell; or a vulvar squamous cell carcinoma cell.

Embodiment A49. A pharmaceutical comprising the bispecific antibody of any of embodiments A1 to A36.

Embodiment A50. A method of treating an individual with cancer comprising administering the pharmaceutical composition of embodiment A49.

Embodiment A51. The method of embodiment A50, wherein the cancer is ductal pancreatic adenocarcinoma, ovarian clear cell adenocarcinoma, colon adenocarcinoma, lung adenocarcinoma cell, or vulvar squamous cell carcinoma.

Embodiment B1. A bispecific antibody comprising a CD47-binding domain and an EGFR binding domain.

Embodiment B2. The bispecific antibody of embodiment B 1, further comprising an Fc domain.

Embodiment B3. The bispecific antibody of embodiment B1 or B2, wherein the bispecific antibody has a higher affinity for CD47 expressed on the surface of a tumor cell than for CD47 expressed on the surface of a red blood cell or a platelet.

Embodiment B4. The bispecific antibody of embodiment B3, wherein the tumor cell expresses EGFR.

Embodiment B5. The bispecific antibody of any of embodiments B1 to B4, wherein a concentration of the bispecific antibody required for half-maximal binding to a human red blood cell is greater than 500 nM.

Embodiment B6. The bispecific antibody of any one of embodiments B1 to B5, wherein bispecific antibody binds to human CD47 with a KD of less than 100 nM.

Embodiment B7. The bispecific antibody of embodiment B6, wherein the bispecific antibody binds to human CD47 with a KD of between 1 nM and 100 nM, between 1 nM and 50 nM, or between 5 nM and 50 nM.

Embodiment B8. The bispecific antibody of any of embodiments B1 to B7, wherein the bispecific antibody binds to EGFR with a KD of less than 25 nM.

Embodiment B9. The bispecific antibody of embodiment B8, wherein the bispecific antibody binds to EGFR with a KD of between 0.2 nM and 25 nM, between 0.2 nM and 10 nM, between 0.2 nM and 2 nM, or between 2 nM and 10 nM.

Embodiment B10. The bispecific antibody of any of embodiments B6 to B9, wherein the KD is determined by surface plasmon resonance.

Embodiment B11. The bi specific antibody of any of embodiments B1 to B10, wherein the CD47-binding domain is a human or engineered human CD47-binding domain.

Embodiment B12. The bispecific antibody of any of embodiments B1 to B11, wherein the CD47-binding domain comprises a heavy chain variable domain and a light chain variable domain.

Embodiment B13. The bi specific antibody of any of embodiments B1 to B11, wherein the CD47-binding domain comprises an scFv.

Embodiment B14. The bispecific antibody of any of embodiments B1 to B13, wherein the EGFR-binding domain comprises a heavy chain variable domain and a light chain variable domain.

Embodiment B15. The bispecific antibody of any of embodiments B1 to B13, wherein the EGFR-binding domain comprises an scFv.

Embodiment B16. The bispecific antibody of any of embodiments B1 to B15, wherein the Fc domain is a human Fc domain.

Embodiment B17. The bispecific antibody of embodiment B16, wherein the isotype of the human Fc domain is IgG1 or IgG4.

Embodiment B18. The bispecific antibody of any of embodiments B1 to B17, wherein the Fc domain is a heterodimeric Fc domain, wherein the heterodimeric Fc region comprises a knob chain and a hole chain, forming a knob-into-hole (KiH) structure.

Embodiment B19. The bispecific antibody of embodiment B18, wherein the knob chain comprises the mutation T366W and the hole chain comprises the mutations T366S, L368A, and Y407V, wherein amino acid position numbering is according to the EU index of Kabat et al.

Embodiment B20. The bispecific antibody of any of embodiments B1 to B19, wherein the bispecific antibody has an asymmetric three-chain knob-into-hole structure.

Embodiment B21. The bispecific antibody of embodiment B20, wherein the CD47-binding domain is an scFv.

Embodiment B22. The bispecific antibody of embodiment B20, wherein the EGFR-binding domain is an scFv.

Embodiment B23. The bispecific antibody of any of embodiments B1 to B22, wherein the CD47-binding domain comprises three heavy chain (HC) complementarity determining regions (CDRs): HC-CDR1, HC-CDR2, and HC-CDR3; and three light chain (LC) CDRs: LC-CDR1, the LC-CDR2, and the LC-CDR3; wherein HC-CDR1 comprises SEQ ID NO: 15, HC-CDR2 comprises SEQ ID NO: 16, HC-CDR3 comprises a sequence with at least 50% identity to SEQ ID NO: 17, LC-CDR1 comprises SEQ ID NO: 18, LC-CDR2 comprises SEQ ID NO: 19, and LC-CDR3 comprises a sequence with at least 50% identity to SEQ ID NO: 20.

Embodiment B24. The bispecific antibody of embodiment B23, wherein HC-CDR3 comprises amino acid substitutions at one or more of K94, E95, G96, S97, F98, G99, V100b, D101, and P102; and LC-CDR3 comprises amino acid substitutions at one or more of Y89, S90, T91, D92, 193, S94, G95, N95a, H95b, W96, and V97.

Embodiment B25. The bispecific antibody of any of embodiments B1 to B24, wherein the heavy chain variable domain of the CD47-binding domain comprises a sequence with at least 90% identity to SEQ ID NO: 5, SEQ ID NO: 10, or SEQ ID NO: 13 and the light chain of the CD47-binding domain comprises a sequence with at least 90% identity to SEQ ID NO: 8, SEQ ID NO: 11, or SEQ ID NO: 14.

Embodiment B26. The bispecific antibody of any of embodiments B1 to B25, wherein the EGFR-binding domain comprises three heavy chain (HC) complementarity determining regions (CDRs), HC-CDR1, HC-CDR2, and HC-CDR3 and three light chain (LC) CDRs, LC-CDR1, the LC-CDR2, and the LC-CDR3; wherein the CDRs are defined by an ordered set of sequences listed as HC-CDR1, HC-CDR2, HC-CDR3, LC-CDR1, LC-CDR2, LC-CDR3; and wherein the CDRs are selected from the group consisting of sequences having at least 90% identity to:

SEQ ID NO: 121, SEQ ID NO: 122, SEQ ID NO: 123, SEQ ID NO: 124, SEQ ID NO: 125, and SEQ ID NO: 126.

Embodiment B27. The bispecific antibody of any of embodiments B1 to B26, wherein the EGFR-binding domain comprises:

a sequence at least 90% identical to SEQ ID NO: 127 and

a sequence at least 90% identical to SEQ ID NO: 128.

Embodiment B28. The bispecific antibody of any of embodiments B1 to B27, wherein less than 1 nM or less than 0.01 nM of the bispecific antibody increases a percentage of A431 cells engulfed by macrophage by at least 4-fold compared to a nonspecific IgG1 antibody control.

Embodiment B29. The bispecific antibody of embodiment B28, wherein a concentration of the antibody required to mediate antibody-dependent cellular phagocytosis of an EGFR-positive, CD47-positive tumor cell by a macrophage is between 0.01 nM and −3 nM.

Embodiment B30. The bispecific antibody of embodiment B29, wherein the EGFR-positive, CD47-positive tumor cell is an OVISE cell or an A431 cell.

Embodiment B31. The bispecific antibody of embodiment B29, wherein the EGFR-positive, CD47-positive tumor cell is selected from epidermoid carcinoma cell, colorectal adenocarcinoma cell, pancreas adenocarcinoma cell, lung squamous cell carcinoma cell, or gastric carcinoma cell.

Embodiment B32. The bispecific antibody of any of embodiments B1 to B28, wherein 100 nM of the bispecific antibody inhibits the binding of SIRPα to CD47 on the surface of a cell by at least 30%.

Embodiment B33. The bispecific antibody of embodiment B32, wherein the cell is a CD47+ EGFR+ tumor cell.

Embodiment B34. The bispecific antibody of embodiment B33, wherein the cell expresses at least as many EGFR proteins on its surface as an HCC-44 cell.

Embodiment B35. The bispecific antibody of embodiment B33, wherein the cell expresses at least 100,000, at least 300,000, at least 600,000 or at least 1,500,000 EGFR proteins on its surface.

Embodiment B36. The bispecific antibody of any of embodiments B1 to B33, wherein 400 nM of the bispecific antibody does not induce hemolysis of red blood cells in a hemagglutination assay.

Embodiment B37. A complex comprising the bispecific antibody of any of embodiments B1 to B36 and a CD47+ EGFR+ target cell.

Embodiment B38. The complex of embodiment B37, wherein the target cell expresses at least as many EGFR proteins on its surface as an HCC-44 cell.

Embodiment B39. The complex of embodiment B37, wherein the target cell expresses at least 100,000, at least 300,000, at least 600,000 or at least 1,500,000 EGFR proteins on its surface.

Embodiment B40. The complex of any of embodiments B37 to B39, wherein the CD47+ EGFR+ target cell is a cancer cell.

Embodiment B41. The complex of embodiment B40, wherein the cancer cell is epidermoid carcinoma cell, colorectal adenocarcinoma cell, pancreas adenocarcinoma cell, lung squamous cell carcinoma, or gastric carcinoma cell.

Embodiment B42. A method of inducing phagocytosis of a CD47+ EGFR+ target cell comprising administering the bispecific antibody of any of embodiments B1 to B36.

Embodiment B43. The method of embodiment B42, wherein the bispecific antibody is administered at a concentration of less than 5 nM, less than 2 nM, less than 1 nM, less than 0.5 nM, less than 0.2 nM, less than 0.1 nM, or less than 0.05 nM.

Embodiment B44. A method of killing a CD47+ EGFR+ target cell comprising administering the bispecific antibody of any of embodiments B1 to B36, wherein the bispecific antibody inhibits the binding of SIRPα to CD47 on the surface of the CD47+ EGFR+ target cell.

Embodiment B45. A method of killing a CD47+ EGFR+ target cell comprising administering the bispecific antibody of any of embodiments B1 to B36, wherein the bispecific antibody induces antibody-dependent cellular cytotoxicity that kills the CD47+ EGFR+ target cell.

Embodiment B46. The method of embodiment B45, wherein the antibody is administered at a concentration of between 0.01-1 nM, between 0.01-0.5 nM, between 0.01-0.25 nM, between 0.01-0.1 nM, between 0.01-0.05 nM, or less than 0.01 nM.

Embodiment B47. The method of embodiment B45 or B46, wherein the CD47+ EGFR+ target cell is selected from an A431 cell, a NCI-H747 cell, an ASPC-1 cell, an EBC-1 cell, or a SNU-5 cell.

Embodiment B48. The method of embodiment B45 or B46, wherein the CD47+ EGFR+ target cell is selected from an epidermoid carcinoma cell, a colorectal adenocarcinoma cell, a pancreas adenocarcinoma cell, a lung squamous cell carcinoma cell, or a gastric carcinoma cell.

Embodiment B49. A pharmaceutical comprising the bispecific antibody of any of embodiments B1 to B36.

Embodiment B50. A method of treating an individual with cancer comprising administering the pharmaceutical composition of embodiment B49.

Embodiment B51. The method of embodiment B50, wherein the cancer is epidermoid carcinoma, colorectal adenocarcinoma, pancreas adenocarcinoma, lung squamous cell carcinoma, or gastric carcinoma.

Embodiment C1. A bispecific antibody comprising a CD47-binding domain and a tumor-associated antigen-binding domain, wherein the CD47-binding domain comprises three heavy chain (HC) complementarity determining regions (CDRs): HC-CDR1, HC-CDR2, and HC-CDR3; and three light chain (LC) CDRs: LC-CDR1, the LC-CDR2, and the LC-CDR3; wherein HC-CDR1 comprises SEQ ID NO: 15, HC-CDR2 comprises SEQ ID NO: 16, HC-CDR3 comprises a sequence with at least 50% identity to SEQ ID NO: 17, LC-CDR1 comprises SEQ ID NO: 18, LC-CDR2 comprises SEQ ID NO: 19, and LC-CDR3 comprises a sequence with at least 50% identity to SEQ ID NO: 20.

Embodiment C2. The bispecific antibody of embodiment C1, wherein the tumor-associated antigen-binding domain binds to a tumor associated antigen comprising ACVR2, HER2/neu, CD20, EGFR, CD3, CD22, CD80, CD23, EpCAM, CD2, CD3, CD19, mesothelin, Mum-1, β-catenin, CDK4, p53, Ras, CDC27, α-actinin-4, TRP1/gp75, Wilm, EphA3, prostatic acid phosphatase (PAP), alpha-fetoprotein (AFP), 9D7, Cyclin-B₁, carcinoembryonic antigen (CEA), gp100/pmel17, BRCA1/2, VEGFR, TGF-βRII, MUC-1, epithelial tumor antigen (ETA), tyrosinase, melanoma associated antigen (MAGE), carbonic anhydrase IX, cytotoxic T-lymphocyte antigen 4, Folate-Binding Protein A-33, prostate specific antigen (PSA), survivin, EGFRvIII, melanocyte derived peptide, multiple melanoma-associated peptides, cervical carcinoma antigen HPV-16-E7, PRAME, SSX-2, CA125, MART, CS-1, BING-4, fibronectin, CML66, MC1R, calcium-activated chloride channel 2, immature laminin receptor, and hTERT.

Embodiment C3. The bispecific antibody of embodiment C1 or C2, wherein the bispecific antibody has a higher affinity for CD47 expressed on the surface of a tumor cell than for CD47 expressed on the surface of a red blood cell or a platelet.

Embodiment C4. The bispecific antibody of embodiment C3, wherein the bispecific antibody further comprises an Fc domain.

Embodiment C5. The bispecific antibody of any of embodiments C1 to C4, wherein a concentration of the bispecific antibody required for half-maximal binding to a human red blood cell is greater than 500 nM.

Embodiment C6. The bispecific antibody of any one of embodiments C1 to C5, wherein bispecific antibody binds to human CD47 with a KD of less than 100 nM.

Embodiment C7. The bispecific antibody of embodiment C6, wherein the bispecific antibody binds to human CD47 with a KD of between 1 nM and 100 nM, between 1 nM and 50 nM, or between 5 nM and 50 nM.

Embodiment C8. The bispecific antibody of any of embodiments C2 to C7, wherein the bispecific antibody binds to the tumor associated antigen with a KD of less than 500 nM.

Embodiment C9. The bispecific antibody of embodiment C8, wherein the bispecific antibody binds to the tumor associated antigen with a KD of between 0.2 nM and 500 nM, between 1 nM and 300 nM, between 5 nM and 200 nM, or between 10 nM and 150 nM.

Embodiment C10. The bispecific antibody of any of embodiments C6 to C9, wherein the KD is determined by surface plasmon resonance.

Embodiment C11. The bispecific antibody of any of embodiments C1 to C10, wherein the CD47-binding domain is a human or engineered human CD47-binding domain.

Embodiment C12. The bispecific antibody of any of embodiments C1 to C11, wherein the CD47-binding domain comprises a heavy chain variable domain and a light chain variable domain.

Embodiment C13. The bispecific antibody of any of embodiments C1 to C11, wherein the CD47-binding domain comprises an scFv.

Embodiment C14. The bispecific antibody of any of embodiments C1 to C13, wherein the tumor associated antigen-binding domain comprises a heavy chain variable domain and a light chain variable domain.

Embodiment C15. The bispecific antibody of any of embodiments C1 to C13, wherein the tumor associated antigen-binding domain comprises an scFv.

Embodiment C16. The bispecific antibody of any of embodiments C1 to C15, wherein the Fc domain is a human Fc domain.

Embodiment C17. The bispecific antibody of embodiment C16, wherein the isotype of the human Fc domain is IgG1, IgG2, or IgG4.

Embodiment C18. The bispecific antibody of any of embodiments C1 to C17, wherein the Fc domain is a heterodimeric Fc domain, wherein the heterodimeric Fc region comprises a knob chain and a hole chain, forming a knob-into-hole (KiH) structure.

Embodiment C19. The bispecific antibody of embodiment C18, wherein the knob chain comprises the mutation T366W and the hole chain comprises the mutations T366S, L368A, and Y407V, wherein amino acid position numbering is according to the EU index of Kabat et al.

Embodiment C20. The bispecific antibody of any of embodiments C1 to C19, wherein the bispecific antibody has an asymmetric three-chain knob-into-hole structure.

Embodiment C21. The bispecific antibody of embodiment C20, wherein the CD47-binding domain is an scFv.

Embodiment C22. The bispecific antibody of embodiment C20, wherein the tumor associated antigen-binding domain is an scFv.

Embodiment C23. The bispecific antibody of any of embodiments C1 to C22, wherein the tumor-associated antigen comprises: ACVR2, HER2/neu, CD20, EGFR, CD3, EphA3, and EpCAM.

Embodiment C24. The bispecific antibody of any of embodiments C1 to C23, wherein HC-CDR3 comprises amino acid substitutions at one or more of K94, E95, G96, S97, F98, G99, V100b, D101, and P102; and LC-CDR3 comprises amino acid substitutions at one or more of Y89, S90, T91, D92, 193, S94, G95, N95a, H95b, W96, and V97.

Embodiment C25. The bispecific antibody of any of embodiments C1 to C24, wherein the heavy chain variable domain of the CD47-binding domain comprises a sequence with at least 90% identity to SEQ ID NO: 5, SEQ ID NO: 10, or SEQ ID NO: 13 and the light chain of the CD47-binding domain comprises a sequence with at least 90% identity to SEQ ID NO: 8, SEQ ID NO: 11, or SEQ ID NO: 14.

Embodiment C26. The bispecific antibody of any of embodiments C23 to C25, wherein the tumor-associated antigen-binding domain comprises three heavy chain (HC) complementarity determining regions (CDRs), HC-CDR1, HC-CDR2, and HC-CDR3 and three light chain (LC) CDRs, LC-CDR1, the LC-CDR2, and the LC-CDR3; wherein the CDRs are defined by an ordered set of sequences listed as HC-CDR1, HC-CDR2, HC-CDR3, LC-CDR1, LC-CDR2, LC-CDR3; and wherein the CDRs are selected from the group consisting of sequences having at least 90% identity to:

SEQ ID NO: 45, SEQ ID NO: 46, SEQ ID NO: 47, SEQ ID NO: 81, SEQ ID NO: 82, and SEQ ID NO: 83;

SEQ ID NO: 48, SEQ ID NO: 49, SEQ ID NO: 50, SEQ ID NO: 84, SEQ ID NO: 85, and SEQ ID NO: 86;

SEQ ID NO: 51, SEQ ID NO: 52, SEQ ID NO: 53, SEQ ID NO: 87, SEQ ID NO: 88, and SEQ ID NO: 89;

SEQ ID NO: 54, SEQ ID NO: 55, SEQ ID NO: 56, SEQ ID NO: 90, SEQ ID NO: 91, and SEQ ID NO: 92;

SEQ ID NO: 57, SEQ ID NO: 58, SEQ ID NO: 59, SEQ ID NO: 93, SEQ ID NO: 94, and SEQ ID NO: 95;

SEQ ID NO: 60, SEQ ID NO: 61, SEQ ID NO: 62, SEQ ID NO: 96, SEQ ID NO: 97, and SEQ ID NO: 98;

SEQ ID NO: 63, SEQ ID NO: 64, SEQ ID NO: 65, SEQ ID NO: 99, SEQ ID NO: 100, and SEQ ID NO: 101;

SEQ ID NO: 66, SEQ ID NO: 67, SEQ ID NO: 68, SEQ ID NO: 102, SEQ ID NO: 103, and SEQ ID NO: 104;

SEQ ID NO: 69, SEQ ID NO: 70, SEQ ID NO: 71, SEQ ID NO: 105, SEQ ID NO: 106, and SEQ ID NO: 107;

SEQ ID NO: 72, SEQ ID NO: 72, SEQ ID NO: 74, SEQ ID NO: 108, SEQ ID NO: 109, and SEQ ID NO: 110;

SEQ ID NO: 75, SEQ ID NO: 76, SEQ ID NO: 77, SEQ ID NO: 111, SEQ ID NO: 112, and SEQ ID NO: 113; and

SEQ ID NO: 78, SEQ ID NO: 79, SEQ ID NO: 80, SEQ ID NO: 114, SEQ ID NO: 115, and SEQ ID NO: 116.

Embodiment C27. The bispecific antibody of any of embodiments C1 to C26, wherein the tumor-associated antigen-binding domain comprises:

a sequence at least 90% identical to SEQ ID NO: 21 and

a sequence at least 90% identical to SEQ ID NO: 33;

a sequence at least 90% identical to SEQ ID NO: 22 and

a sequence at least 90% identical to SEQ ID NO: 34; or

a sequence at least 90% identical to SEQ ID NO: 24 and

a sequence at least 90% identical to SEQ ID NO: 36.

Embodiment C28. The bispecific antibody of any of embodiments C1 to C25, wherein the tumor-associated antigen-binding domain comprises three heavy chain (HC) complementarity determining regions (CDRs), HC-CDR1, HC-CDR2, and HC-CDR3 and three light chain (LC) CDRs, LC-CDR1, the LC-CDR2, and the LC-CDR3; wherein the CDRs are defined by an ordered set of sequences listed as HC-CDR1, HC-CDR2, HC-CDR3, LC-CDR1, LC-CDR2, LC-CDR3; and wherein the CDRs are selected from the group consisting of sequences having at least 90% identity to:

SEQ ID NO: 121, SEQ ID NO: 122, SEQ ID NO: 123, SEQ ID NO: 124, SEQ ID NO: 125, and SEQ ID NO: 126.

Embodiment C29. The bispecific antibody of any of embodiments C1 to C25, wherein the tumor-associated antigen-binding domain comprises:

a sequence at least 90% identical to SEQ ID NO: 127 and

a sequence at least 90% identical to SEQ ID NO: 128.

Embodiment C30. The bispecific antibody of any of embodiments C1 to C29, wherein less than 1 nM or less than 0.01 nM of the bispecific antibody increases a percentage of A431 cells engulfed by macrophage by at least 4-fold compared to a nonspecific IgG1 antibody control.

Embodiment C31. A pharmaceutical comprising the bispecific antibody of any of embodiments C1 to C30.

Embodiment C32. A method of treating an individual with cancer comprising administering the pharmaceutical composition of embodiment C31.

### 5. EXAMPLES

These examples are provided for illustrative purposes only and not to limit the scope of the claims provided herein.

### 5.1 EXAMPLE 1. BENCHMARK ANTIBODIES AND REAGENTS

Sequences of the benchmark antibodies and reagents used in this study were parsed from patent literature and cloned into a human IgG1 backbone as indicated in Table 1.

TABLE 1
Benchmark reagents.

| | | Parent | Source of parent |
|---|---|---|---|
| Name | Description | molecule | molecule |
| CD47 BMK-1 | αCD47 IgG1 | 5F9 | Forty Seven Inc. |
| CD47 BMK-2 | αCD47 IgG1 | 2A1 | Celgene |
| CD47 BMK-3 | αCD47 IgG1 | 13H3 | I-Mab |
| CD47 BMK-4 | SIRPα- Fc fusion | TTI-621 | Trillium |
| | | | Therapeutics |
| EpCAM BMK-5 | αEpCAM IgG1 | VB6-845; | Sesen Bio |
| | | Vicinium | |
| EpCAM BMK-6 | αEpCAM IgG1 | 3622 | GlaxoSmithKline |

### 5.2 EXAMPLE 2. CD47+ EPCAM+ CELL LINES

Tumor derived cell lines expressing CD47 and/or EpCAM were obtained from the American Type Culture Collection (ATCC) and were grown in culture medium containing 10% fetal bovine serum in a 37° C. incubator with 5% CO2. CFPAC-1 cells were cultured in IMDM medium, Raji cells were cultured in RPMI-1640 medium, and A431 cells were cultured in DMEM medium.

Cell surface expression of CD47 and EpCAM was measured by flow cytometry. Tumor cells were harvested, centrifuged and resuspended in FACS buffer (PBS+2% FBS) at a concentration of 1×10⁶ cells/mL. 100 μL of cell suspension was dispensed into each well of a 96-well plate, 100 μL of CD47 BMK-1 or EpCAM BMK-6 at 10 μg/mL was added to the wells and incubated for 1 hour at 4° C. The cells were washed three times with FACS buffer. After the third wash, the cells were resuspended in 100 μL 1: 500 diluted AlexaFluor-488 mouse anti-Human IgG1 Fc secondary antibody (Invitrogen, Cat #A10631) and incubated for 1 hour at 4° C. in the dark. The cells were washed three times with 200 μL PBS by centrifuging at 2000 RPM for 5 minutes.

After the last wash, the cells were resuspended in 300 μL cold PBS and analyzed on a FACSVerse™ (BD Biosciences) flow cytometer. Table 2 shows the relative cell surface expression levels of CD47 and EpCAM in these cell lines.

TABLE 2
Cancer cell lines used in this study and
their CD47 and EpCAM expression levels.

| | | CD47 | EpCAM |
|---|---|---|---|
| | | expression MFI | expression MFI |
| Cell | | (Receptors/ | (Receptors/ |
| line | Origin | cell*) | cell\hat{ }) |
| CFPAC-1 | Ductal pancreatic | 76,752 | 71,100 |
| | adenocarcinoma | (N/A) | (N/A) |
| OVISE | Ovarian clear cell | 49,742 | 252,006 |
| | adenocarcinoma | (N/A) | (N/A) |
| CW-2 | Colon | 13,080 | 314,203 |
| | adenocarcinoma | (N/A) | (N/A) |
| HCC44 | Lung | 15,116 | 7,346 |
| | adenocarcinoma | (N/A) | (N/A) |
| Raji | Burkitt lymphoma | 39,976 | 1,547 |
| | | (117,640) | (N/A) |
| SKOV3 | Ovarian | 17,794 | 13,799 |
| | adenocarcinoma | (N/A) | (317,138) |
| A431 | Vulvar squamous | 14,008 | 28,228 |
| | cell carcinoma | (N/A) | (1,500,000) |

N/A: not available
*Puro et. al., Molecular Cancer Therapeutics, published online Dec. 26, 2016.
\hat{ }Casaletto et. al., PNAS 116(15) 7533-7542, March, 2019.

The human CD47 stable cell lines were generated by transfecting CHOK1 cells with the pLVX-IRES-Puro lentiviral expression vector (Clontech, Cat. No 632183) encoding full length human CD47 genes, respectively.

### 5.3 EXAMPLE 3. BISPECIFIC CD47×EPCAM ANTIBODIES INDUCE PHAGOCYTOSIS OF CD47+ EPCAM+ CELLS

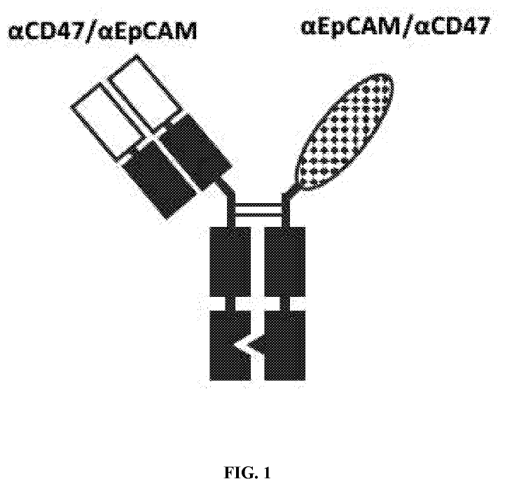

A bispecific antibody was constructed with a CD47-binding domain derived from CD47 BMK-2, an EpCAM-binding scFv domain derived from EpCAM BMK-5, and an IgG1 Fc domain with an asymmetric knob-into-hole structure that inhibits the formation of heavy chain dimers (Carter, J. Immunol. Protein engineering, vol. 9, no. 7, pp 617-621, 1996). The format of this antibody is referred to as a three-chain knob into hole (3C-KIH) CD47×EpCAM scFv (FIG. 1). Additional bispecific antibody designs are described in Klein et. al., mAbs 4(6) 653-663, 2012. The amino acid sequences of the polypeptide components of this antibody are shown in Table 3.

TABLE 3
Polypeptide chain sequences of the proof-of-
concept CD47 x EpCAM bispecific antibody

| Name | SEQ ID NO | Sequence |
|---|---|---|
| CD47 BMK-2 | 1 | QMQLVQSGAEVKKTGSSVKVSCKAS |
| heavy chain | | GFNIKDYYLHWVRQAPGQALEWMGW |
| knob | | IDPDQGDTEYAQKFQDRVTITRDRS |
| | | MSTAYMELSSLRSEDTAMYYCNAAY |
| | | GSSSYPMDYWGQGTTVTVSSASTKG |
| | | PSVFPLAPSSKSTSGGTAALGCLVK |
| | | DYFPEPVTVSWNSGALTSGVHTFPA |
| | | VLQSSGLYSLSSVVTVPSSSLGTQT |
| | | YICNVNHKPSNTKVDKKVEPKSCDK |
| | | THTCPPCPAPELLGGPSVFLFPPKP |
| | | KDTLMISRTPEVTCVVVDVSHEDPE |
| | | VKFNWYVDGVEVHNAKTKPREEQYN |
| | | STYRVVSVLTVLHQDWLNGKEYKCK |
| | | VSNKALPAPIEKTISKAKGQPREPQ |
| | | VYTLPPSRDELTKNQVSLWCLVKGF |
| | | YPSDIAVEWESNGQPENNYKTTPPV |
| | | LDSDGSFFLYSKLTVDKSRWQQGNV |
| | | FSCSVMHEALHNHYTQKSLSLSPGK |
| CD47 BMK-2 | 2 | NIQMTQSPSAMSASVGDRVTITCKA |
| light chain | | SQDIHRYLSWFQQKPGKVPKHLIYR |
| | | ANRLVSGVPSRFSGSGSGTEFTLTI |
| | | SSLQPEDFATYYCLQYDEFPYTFGG |
| | | GTKVEIKRTVAAPSVFIFPPSDEQL |
| | | KSGTASVVCLLNNFYPREAKVQWKV |
| | | DNALQSGNSQESVTEQDSKDSTYSL |
| | | SSTLTLSKADYEKHKVYACEVTHQG |
| | | LSSPVTKSFNRGEC |
| EpCAM BMK- | 3 | DIQMTQSPSSLSASVGDRVTITCRS |
| 5 scFv | | TKSLLHSNGITYLYWYQQKPGKAPK |
| | | LLIYQMSNLASGVPSRFSSSGSGTD |
| | | FTLTISSLQPEDFATYYCAQNLEIP |
| | | RTFGQGTKVELKRATPSHNSHQVPS |
| | | AGGPTANSGTSGSEVQLVQSGPGLV |
| | | QPGGSVRISCAASGYTFTNYGMNWV |
| | | KQAPGKGLEWMGWINTYTGESTYAD |
| | | SFKGRFTFSLDTSASAAYLQINSLR |
| | | AEDTAVYYCARFAIKGDYWGQGTLL |
| | | TVSSGGSGGSGGASKTHTCPPCPAP |
| | | ELLGGPSVFLFPPKPKDTLMISRTP |
| | | EVTCVVVDVSHEDPEVKFNWYVDGV |
| | | EVHNAKTKPREEQYNSTYRVVSVLT |
| | | VLHQDWLNGKEYKCKVSNKALPAPI |
| | | EKTISKAKGQPREPQVYTLPPSRDE |
| | | LTKNQVSLSCAVKGFYPSDIAVEWE |
| | | SNGQPENNYKTTPPVLDSDGSFFLV |
| | | SKLTVDKSRWQQGNVFSCSVMHEAL |
| | | HNHYTQKSLSLSPGK |

The bispecific antibodies were tested in an antibody-dependent cellular phagocytosis (ADCP) assay. Peripheral blood mononuclear cells (PBMCs) were isolated from human donors. Monocytes were enriched using an Human Monocyte Enrichment Kit without CD16 depletion (STEMCELL, Cat #19058). Isolated monocytes were differentiated into macrophages by culturing monocytes in complete culture media (RPMI 1640+10% FBS) with 20 ng/ml of human Macrophage Colony-Stimulating Factor (M-CSF, Peprotech, Cat #: 3-25-10). The media was changed every three days. After 7 days of culturing in M-CSF containing culture media, macrophages were collected and counted. Target tumor cells were collected and washed with D-PBS two times to remove remaining FBS. The washed tumor cells were resuspended in PBS at a cell concentration of 5-10×10⁶/mL. Cancer cells were stained with CFSE (ebiosciences, Cat #: 65-0580-84) at a final concentration of 3 μM and mixed immediately. The cells were stained in the dark at room temperature for 10 minutes. The staining was terminated by adding 4-5 volumes of cold complete media and incubating on ice for 5 minutes. Stained cells were washed three times with RPMI 1640+10% FBS. Cells were resuspended in 1 ml RPMI 1640+10% FBS, counted, and then diluted or concentrated to 3×10⁵ cells/mL. 50 μl cells were seeded into a 96 well deep U-plate (Axygen, Cat #: P-DW-20-C) wherein each well contained 1.5×10⁴ cells. 50 μl of diluted antibodies were added to each well. 100 μl of macrophages (1.5×10⁴ cells) were added to each well and incubated at 37° C., 5% CO2 for 1.5 hours. After incubation, cells were washed with 2 ml of 2% FBS in D-PBS once. 100 μl of diluted Fc blocker (Human TruStain FcX (Fc Receptor Blocking Solution), Biolegend Cat #: 422302)) was added and the cells were incubated at room temperature for 10 minutes. 20 μl of diluted anti-human CD11b antibody was added to each well and incubated for 30 minutes at 4° C. in the dark. Cells were washed with 2% FBS-D-PBS once. Phagocytosis was detected in a flow cytometer by the appearance of CFSE/CD11b double positive cells indicative of macrophages that engulfed the tumor cells.

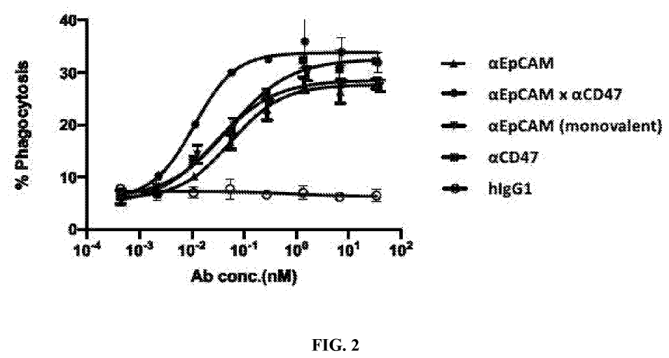

The bispecific antibody promoted phagocytosis of CFPAC-1 pancreatic cancer cells, OVISE ovarian cancer cells, CW-2 colon cancer cells and HCC44 lung cancer cells. Exemplary results for CFPAC-1 cells are presented in FIG. 2. A summary for the four cell lines with additional controls is presented in Table 4.

TABLE 4
Bispecific CD47 × EpCAM antibodies induce antibody-dependent cellular
phagocytosis of CD47+, EpCAM+ cancer cell lines.

| | Antibody features | | | | | |
|---|---|---|---|---|---|---|
| | CD47 | EpCAM | EC50 (nM) | | | |
| Antibody name | Valency | Valency | CFPAC-1 | OVISE | CW-2 | HCC44 |
| Bispecific | Mono | Mono | 0.011 | 0.008 | 0.003 | 0.020 |
| αCD47 × αEpCAM | | | | | | |
| CD47 BMK-2 | Bi | | 0.053 | 0.023 | 0.008 | 0.039 |
| EpCAM BMK-5 | | Bi | 0.055 | 0.009 | 0.004 | 0.025 |
| EpCAM one arm control | | Mono | 0.028 | 0.013 | 0.007 | 0.034 |
| huIgG1 control | | | n.d. | n.d. | n.d. | n.d. |

*n.d.: not detected

In all four cell lines, the bispecific antibody promoted phagocytosis more effectively than a bivalent anti-CD47 antibody with the same anti-CD47 binding domain. A non-specific IgG1 isotype control antibody did not promote phagocytosis of any tested CD47+ cell.

The EpCAM BMK-5 (monovalent and bivalent) also promoted phagocytosis through its IgG1 domain interacting with Fc gamma receptors on phagocytic cells, thereby promoting phagocytosis directly. This effect was more pronounced in OVISE and CW-2 cells, which have high EpCAM expression.

### 5.4 EXAMPLE 4. ENGINEERING AND TESTING ANTI-CD47 BINDING DOMAINS

VIR47 is an anti-CD47 antibody identified from OmniRat® rats. The variable domain of VIR47 was grafted onto a human IgG4 framework (IgG4.SPL) with an 5228P substitution compared to the sequence of native human IgG4. VIR47 efficiently blocks the interaction between CD47 and SIRPα. The VIR47 mAb has relatively weak binding to RBCs, a reduced ability to induce phagocytosis of RBCs, and does not induce hemagglutination of human RBCs. VIR47 is described in PCT/CN2019/113296, which is incorporated by reference in its entirety.

To enhance Fc gamma receptor interactions, the IgG4 Fc domain of VIR47 was replaced by an IgG1 Fc domain. Additionally, the framework region of VIR47 was further engineered by replacing rat-derived amino acids with amino acids found at the corresponding positions in the sequence of the closest human germline ortholog. Two of these optimized variants of VIR47 are known, according to Kabat numbering (EU index of Kabat et al 1991 Sequences of Proteins of Immunological Interest), as VIR47.V7 (HC: F₇₉Y, G_{82b}S; LC: E₁₀₃K) and VIR47.V8 (HC: I₆₇F, F₇₉Y, G_{82b}S; LC: M₄L, E₁₀₃K). The sequences of the heavy and light chains of VIR47 and related mAbs are shown in Table 5. The complementarity determining regions (CDRs) of VIR47 are presented in Table 6.

TABLE 5
Polypeptide sequences of anti-CD47 binding domains derived from VIR47.
CDRs are underlined and bolded.

| Name | SEQ ID NO | Sequence |
|---|---|---|
| VIR47 | 4 | EVQLLESGGGLVQPGGSLRLSCAASGFTFSNFAMSWVRQAP |
| hIgG4-SPL | | GKGLEWVSTISASGGRTFYADSVKGRITISRDNSKNTLFLQ |
| HC | | MNGLRAEDTAVYYCAKEGSFGEGVDPWGQGTLVTVSSAST |
| | | KGPSVFPLAPCSRSTSESTAALGCLVKDYFPEPVTVSWNSGA |
| | | LTSGVHTFPAVLQSSGLYSLSSWTVPSSSLGTKTYTCNVDH |
| | | KPSNTKVDKRVESKYGPPCPPCPAPEFLGGPSVFLFPPKPKD |
| | | TLMISRTPEVTCVVVDVSQEDPEVQFNWYVDGVEVHNAKT |
| | | KPREEQFNSTYRVVSVLTVLHQDWLNGKEYKCKVSNKGLP |
| | | SSIEKTISKAKGQPREPQVYTLPPSQEEMTKNQVSLTCLVKG |
| | | FYPSDIAVEWESNGQPENNYKTTPPVLDSDGSFFLYSRLTVD |
| | | KSRWQEGNVFSCSVMHEALHNHYTQKSLSLSLGK |
| VIR47 | 5 | EVQLLESGGGLVQPGGSLRLSCAASGFTFSNFAMSWVRQAP |
| hIgG4-SPL | | GKGLEWVSTISASGGRTFYADSVKGRITISRDNSKNTLFLQ |
| VH | | MNGLRAEDTAVYYCAKEGSFGEGVDPWGQGTLVTVSS |
| VIR47 | 6 | SYEMTQPPSVSVSPGQTARITCSGDALPKKYAYWYQQKSG |
| LC | | QAPVLVIYEDNKRPSGIPERFSGSSSGTMATLTISGAQVEDE |
| | | ADYYCYSTDISGNHWVFGGGTELTVLGQPKAAPSVTLFPPS |
| | | SEELQANKATLVCLISDFYPGAVTVAWKADSSPVKAGVETT |
| | | TPSKQSNNKYAASSYLSLTPEQWKSHRSYSCQVTHEGSTVE |
| | | KTVAPTECS |
| VIR47 | 7 | EVQLLESGGGLVQPGGSLRLSCAASGFTFSNFAMSWVRQAP |
| hIgG1 | | GKGLEWVSTISASGGRTFYADSVKGRITISRDNSKNTLFLQ |
| HC | | MNGLRAEDTAVYYCAKEGSFGEGVDPWGQGTLVTVSSAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPELLGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVS |
| | | NKALPAPIEKTISKAKGQPREPQVYTLPPSRDELTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGK |
| VIR47 | 8 | SYEMTQPPSVSVSPGQTARITCSGDALPKKYAYWYQQKSG |
| LC | | QAPVLVIYEDNKRPSGIPERFSGSSSGTMATLTISGAQVEDE |
| | | ADYYCYSTDISGNHWVFGGGTELTVLGQPKAAPSVTLFPPS |
| | | SEELQANKATLVCLISDFYPGAVTVAWKADSSPVKAGVETT |
| | | TPSKQSNNKYAASSYLSLTPEQWKSHRSYSCQVTHEGSTVE |
| | | KTVAPTECS |
| VIR47.V7 | 9 | EVQLLESGGGLVQPGGSLRLSCAASGFTFSNFAMSWVRQAP |
| hIgG1 | | GKGLEWVSTISASGGRTFYADSVKGRITISRDNSKNTLYLQ |
| HC | | MNSLRAEDTAVYYCAKEGSFGEGVDPWGQGTLVTVSSAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPELLGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVS |
| | | NKALPAPIEKTISKAKGQPREPQVYTLPPSRDELTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGK |
| VIR47.V7 | 10 | EVQLLESGGGLVQPGGSLRLSCAASGFTFSNFAMSWVRQAP |
| hIgG1 | | GKGLEWVSTISASGGRTFYADSVKGRITISRDNSKNTLYLQ |
| VH | | MNSLRAEDTAVYYCAKEGSFGEGVDPWGQGTLVTVSS |
| VIR47.V7 | 11 | SYEMTQPPSVSVSPGQTARITCSGDALPKKYAYWYQQKSG |
| LC | | QAPVLVIYEDNKRPSGIPERFSGSSSGTMATLTISGAQVEDE |
| | | ADYYCYSTDISGNHWVFGGGTKLTVLGQPKAAPSVTLFPP |
| | | SSEELQANKATLVCLISDFYPGAVTVAWKADSSPVKAGVET |
| | | TTPSKQSNNKYAASSYLSLTPEQWKSHRSYSCQVTHEGSTV |
| | | EKTVAPTECS |
| VIR47.V8 | 12 | EVQLLESGGGLVQPGGSLRLSCAASGFTFSNFAMSWVRQAP |
| hIgG1 | | GKGLEWVSTISASGGRTFYADSVKGRFTISRDNSKNTLYLQ |
| HC | | MNSLRAEDTAVYYCAKEGSFGEGVDPWGQGTLVTVSSAST |
| | | KGPSVFPLAPSSKSTSGGTAALGCLVKDYFPEPVTVSWNSG |
| | | ALTSGVHTFPAVLQSSGLYSLSSVVTVPSSSLGTQTYICNVN |
| | | HKPSNTKVDKKVEPKSCDKTHTCPPCPAPELLGGPSVFLFPP |
| | | KPKDTLMISRTPEVTCVVVDVSHEDPEVKFNWYVDGVEVH |
| | | NAKTKPREEQYNSTYRVVSVLTVLHQDWLNGKEYKCKVS |
| | | NKALPAPIEKTISKAKGQPREPQVYTLPPSRDELTKNQVSLT |
| | | CLVKGFYPSDIAVEWESNGQPENNYKTTPPVLDSDGSFFLY |
| | | SKLTVDKSRWQQGNVFSCSVMHEALHNHYTQKSLSLSPGK |
| VIR47.V8 | 13 | EVQLLESGGGLVQPGGSLRLSCAASGFTFSNFAMSWVRQAP |
| hIgG1 | | GKGLEWVSTISASGGRTFYADSVKGRFTISRDNSKNTLYLQ |
| VH | | MNSLRAEDTAVYYCAKEGSFGEGVDPWGQGTLVTVSS |
| VIR47.V8 | 14 | SYELTQPPSVSVSPGQTARITCSGDALPKKYAYWYQQKSG |
| LC | | QAPVLVIYEDNKRPSGIPERFSGSSSGTMATLTISGAQVEDE |
| | | ADYYCYSTDISGNHWVFGGGTKLTVLGQPKAAPSVTLFPP |
| | | SSEELQANKATLVCLISDFYPGAVTVAWKADSSPVKAGVET |
| | | TTPSKQSNNKYAASSYLSLTPEQWKSHRSYSCQVTHEGSTV |
| | | EKTVAPTECS |

TABLE 6
CDR sequences of VIR47.

| | SEQ | | SEQ | | SEQ | |
|---|---|---|---|---|---|---|
| | ID | | ID | | ID | |
| CHAIN | NO | CDR1 | NO | CDR2 | NO | CDR3 |
| Heavy | 15 | NFAMS | 16 | TISASGGRT | 17 | EGSFGE |
| | | | | FYADSVKG | | GVDP |
| Light | 18 | SGDAL | 19 | EDNKRPS | 20 | YSTDIS |
| | | PKKYAY | | | | GNHWV |

The IgG1 form and engineered variants were characterized by an ELISA-based binding assay, flow cytometry quantification of binding to CD47-expressing cells, and measurement of binding kinetics by surface plasmon resonance with a Biacore instrument.

### 5.4.1 Generation of Human CD47

DNA encoding the extracellular domains (ECD) of human CD47 were cloned into pRK5 (ATCC Cat #209784). The resulting constructs with C-terminal 6×His tags (SEQ ID NO: 129) were transfected into Expi293F cells. After 72 hours, CD47-expressing cells were harvested by centrifugation for 5 minutes at 2000 rpm at 4° C. The supernatant was collected. Ni-NTA (Qiagen, Cat #30410) resin was pre-equilibrated with buffer A (137 mM NaCl, 2.7 mM KCl, 10 mM Na2HPO4, 2 mM KH2PO4, pH 7.4) and incubated with the supernatant for 2 hours at 4° C. on a rotator. The resin was filled in the Ni Sepharose excel (GE, Cat #GE17371201) and washed with buffer A until no signal (0D595, about 20-30 column volumes (CV) was observed by Coomassie-Brilliant Blue G-250. The target protein was eluted using buffer B (137 mM NaCl, 2.7 mM KCl, 10 mM Na₂HPO₄, 2 mM KH₂PO₄, pH 7.4, 250 mM imidazole) for 3 column volumes (CV). The SuperdexTX 200 increase column (GE, Cat #GE28-9909-44) was pre-equilibrated by buffer A, then the eluate was loaded onto the column. The column was washed with buffer A and fractions were collected. Different fractions were resolved on a 12% SDS-PAGE and desired fractions were combined and neutralized with buffer C (137 mM NaCl, 2.7 mM KCl, 10 mM Na₂HPO₄, 2 mM KH₂PO₄, pH 7.4). The target protein was concentrated by using an ultra-filtration tube (Amicon, Cat #42409) with a molecular cutoff of 30 kDa, then aliquoted and snap frozen using liquid N2 and stored at −80° C.

### 5.4.2 Antibody Binding to Antigen Measured by ELISA

A 96-well plate was coated overnight at 4° C. with 1 μg/ml recombinant CD47. After washing 3 times, the plate was blocked with 300 μl 1% BSA in PBST at 37° C. for 1 hour. Serially diluted antibodies were added and incubated at 37° C. for 1 hour. The plate was then washed 4 times with PBST and incubated with 1:5000 diluted peroxidase labeled goat anti-human IgG (Fab specific) secondary antibody (Sigma, Cat #A0293) for 1 hour at 37° C. The plate was washed again 4 times with PBST, incubated with 3,3′,5,5′-tetramethylbenzidine (TMB) substrate for 15 min at room temperature, terminated with 1N HCl, and then read at 450 nm. VIR47 and its IgG1, V7 and V8 variants had comparable binding to human CD47 by ELISA.

### 5.4.3 Antibody Binding to Cells Measured by Flow Cytometry

Antibody binding to CD47+ cell lines was quantified by flow cytometry. On cells, the CD47 ECD is presented in its natural orientation with natural glycosylation. Harvested cells were centrifuged at 2000 rpm for 5 min, resuspended in 10-15 ml ice-cold culture medium, and then counted. Cells were resuspended in blocking buffer (PBS plus 2% FBS) at a concentration of 3×10⁶ cells/mL. 100 μL of the cell suspension was dispensed into each well of a 96-well plate. Purified antibodies were diluted to the desired concentrations with blocking buffer and 100 μL of diluted antibodies were added to the well and incubated for 1 hour at 4° C. The cells were then washed 3 times with PBS plus 2% FBS. After the third wash, the cells were resuspended in 100 μL 1:500 diluted Alexa Fluor 488 labeled Mouse anti-Human IgG1 Fc secondary antibody (Invitrogen, Cat #: A10631) and incubated for 1 hour at 4° C. in the dark. The cells were then washed 3 times with 200 μL PBS by centrifuging at 2000 rpm for 5 min. After the last wash, the cells were resuspended in 300 μL cold PBS and analyzed on a FACSVerse™ (BD Biosciences) flow cytometer.

In a flow cytometry assay, VIR47 and its IgG1, V7 and V8 variants bound to CHO cells expressing human CD47 with comparable affinity.

### 5.4.4 Surface Plasmon Resonance Binding Assay

The binding kinetics of the anti-CD47 antibodies were evaluated on a Biacore 8K instrument (GE Healthcare). Biacore Series S CM5 sensor chips were immobilized with monoclonal mouse anti-human IgG (Fc) antibody (human antibody capture kit from GE Healthcare). Antibodies were captured on each flow cell. Serial 3-fold dilutions of each antigen were injected at a flow rate of 30 μl/min. Each sample was analyzed with 1 min association and 10 min dissociation at room temperature (25° C.). After each injection, the chip was regenerated using 3M MgCl2. A 1:1 Langmuir model of simultaneous fitting of kₒₙ and k_{off} was used for kinetics analysis. Flowing monomeric antigens over immobilized antibodies yields binding results that are not influenced by antibody valency.

By surface plasmon resonance, VIR47 and its IgG1, V7 and V8 variants had similar binding affinities for human CD47, with KD values between 5 and 50 nM. The KD for CD47 BMK-1 binding to human CD47 was between 1 nM and 5 nM.

### 5.4.5 Antibody-Dependent Cellular Phagocytosis

The ADCP activity of IgG1 forms of VIR47 and its engineered variants was tested on OVISE ovarian cancer cells and compared to a benchmark αCD47 antibody. The three forms of VIR47 had similar ADCP activity to each other, but lower activity than the BMK-1 benchmark.

### 5.4.6 Antibody-Dependent Cellular Cytotoxicity

The ability of anti-CD47 antibodies to mediate antibody-dependent cellular cytotoxicity (ADCC) was tested on CFPAC-1 pancreatic cancer cells, which express high levels of CD47. The cells were washed once with balanced salt solution or culture medium and cell numbers were adjusted to 1×10⁶ cells/ml. 2 μL of BATDA fluorescence enhancing ligand (Perkin Elmer, Cat #C136-100) was then added to each mL of cells and incubated for 20 min at 37° C. in a cell incubator. After incubation, cells were centrifuged, culture medium was aspirated. The labeled cells were washed 4 times with PBS. After the final wash, cells were re-suspended in culture medium and adjusted to 5×10⁴ cell/ml. 200 μL cell suspension was then added to each well of the 96-well plate to make the cell number per well to 1×10⁴. Background release was determined by withdrawing an aliquot of the labeled target cells, centrifuge and supernatant was transferred into an empty well. The reading was background release. 1×10⁴ labeled target cells were transferred to sterile 96-well assay plate. Antibodies were serially diluted with RPMI-1640 containing 10% FBS. 50 μL of serially-diluted antibodies were added to assay plate containing target cell and incubated at 37° C., 5% CO2 for 5-10 min. Effector cells NK92/CD16a176V were harvested and suspended in RPMI-1640 containing 10% FBS. 50 μl/well effector cells were added to each well of assay plate at different effector to target ratios. Set up controls: target spontaneous (target cell+100 μL medium); target maximum (target cell+100 μL medium+10 μL lysis buffer); background (100 μL the labeled target cell supernatant and 100 μL dilution medium). The plates were incubated in a humidified 5% CO2 atmosphere at 37° C. for 2 hours. At the end of incubation, 10 μL of Lysis Buffer (Perkin Elmer, Cat #4005-0010) was added to the maximum release well. The plates were centrifuged for 5 min at 500 g. 20 μL of the supernatant from each well was transferred to a flat-bottom detection plate. 200 μL of Europium Solution (Perkin Elmer, Cat #C135-100) was then added to each well of the detection plate. The plate was shaken at 250 rpm for 15 min at room temperature and the fluorescence was then measured in a time-resolved fluorometer within 5 hrs.

VIR47 and its IgG1, V7 and V8 variants had comparable ADCC activity. As expected, the IgG4 form had essentially no activity.

### 5.4.7 Binding to Red Blood Cells and Hemagglutination

A CD47 binding domain that preferentially binds to the forms of CD47 expressed on tumor cells compared to the forms of CD47 expressed on red blood cells or platelets is desirable to enhance the safety profile of a therapeutic agent.

The RBC binding assay was performed by spinning down fresh human whole blood at 200 g for 10 minutes. Collected RBCs were washed twice with PBS and counted using flow cytometry. 1×10⁶ cells were dispensed into each well of a 96 well culture plate. Serially diluted anti-CD47 antibodies were added and incubated for 1 hour at 4° C. Cells were washed with FACS buffer (PBS+2% FBS) twice. Secondary antibody (Alexa Fluor® 488 Goat Anti-Human IgG (H+L)) was added and incubated for 1 hour at 4° C. Cells were washed twice and resuspended in 200 μl of FACS buffer and analyzed by flow cytometry.

The hemagglutination assay was performed by diluting human red blood cells (RBCs) and incubating RBCs at 37° C. for 2 hours with a titration of CD47 antibodies (from 100 μg/ml) in a round bottom 96 well plate. Hemagglutination is demonstrated by the presence of crosslinked RBCs, which appear as a haze because they do not settle to the bottom of the well, in contrast to non-hemagglutinated RBCs.

VIR47 was selected for its low RBC binding and no hemagglutination activity. The IgG1 forms and engineered variants share these properties. In particular, >20-fold higher concentrations of VIR47 and its variants were required for RBC binding compared to the CD47 BMK-1 benchmark. Furthermore, hemagglutination was not detected for any VIR47 variants, even at the highest tested concentration (666.7 nM).

### 5.5 EXAMPLE 5. ALANINE SCANNING OF VIR47

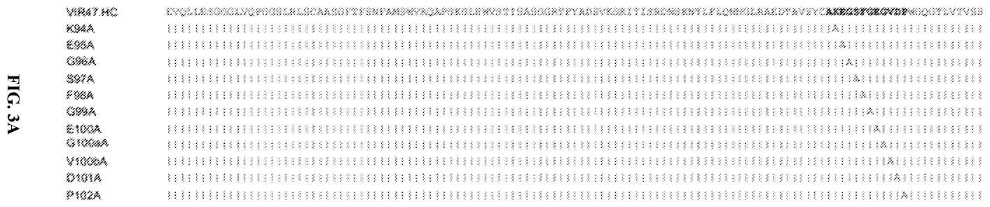

To optimize the binding affinity of the VIR47 anti-CD47 antibody, variants of the IgG1 form of VIR47 were generated by alanine-scanning mutagenesis of the heavy chain and light chain CDR3 domains, as shown in FIG. 3A and FIG. 3B. Binding of these variants to human CD47 was measured by ELISA and compared to the parental VIR47 IgG1 antibody, as shown in Table 7.

TABLE 7
Binding of CDR3 alanine scan variants of αCD47
mAb VIR47 to human CD47 as determined by ELISA.
EC50 ranges for huCD47 - A: 0.01 nM-0.15 nM;
B: 0.15 nM-0.4 nM; C: 0.4 nM-5 nM; D: ≥5 nM.

| | | EC50 |
|---|---|---|
| HC | LC | huCD47 |
| VIR47 IgG1 | | A |
| K94A | | A |
| E95A | | D |
| G96A | | A |
| S97A | | A |
| F98A | | C |
| G99A | | B |
| E100A | | D |
| G100aA | | B |
| V100bA | | B |
| D101A | | B |
| P102A | | B |
| | Y89A | C |
| | S90A | A |
| | T91A | B |
| | D92A | A |
| | I93A | A |
| | S94A | C |
| | G95A | C |
| | N95aA | A |
| | H95bA | A |
| | W96A | A |
| | V97A | A |

### 5.6 EXAMPLE 6. ENGINEERING AND TESTING ANTI-EPCAM BINDING DOMAINS

Anti-EpCAM antibodies were generated by immunizing mice with human EpCAM protein with a C-terminal Fc tag (Novoprotein, Cat #: CM50) combined with cyno-EpCAM protein with a C-terminal Fc tag (Sino Biological, Cat #: 90299-C02H). B cells were used to generate murine hybridomas using the method described in (U.S. Pat. No. 5,597,725). Primary screening was performed by ELISA using human EpCAM with a C-terminal His tag. A secondary screen was performed by ELISA on human EpCAM (Novoprotein, Cat #: C339) with C-terminal His tags. Binding of antibodies was also tested by flow cytometry analysis on CHO-K1 cells expressing human EpCAM or no EpCAM. Hybridomas expressing antibodies specific to human EpCAM were used to generate chimeric antibodies.

### 5.6.1 Sequences of Anti-EpCAM Binding Domains

Chimeric antibodies were generated by fusing an EpCAM-specific murine VH region to a human IgG1 constant region. Binding was assessed by ELISA and cell binding via flow cytometry analysis. The benchmark antibody from these studies was EpCAM BMK-6, Tables 8-11 summarize the polypeptide sequences of the binding domains of the heavy and light chains and its respective CDRs.

TABLE 8
Polypeptide sequences of the anti-EpCAM
heavy chain binding domains. CDRs are
underlined and bolded

| | SEQ | |
|---|---|---|
| | ID | |
| Name | NO | Sequence |
| mAb- | 21 | QVQLQQSGPELVKPGTSVKLSCRASGYTFTSYDINWVKQ |
| 1 | | RPGQGLEWIGWIYPRDGSTKYNERFKGKATLTVDTSSS |
| | | TAYMEFHTLTSEDSAVYFCARDPYYYGTIYGYFDVWGT |
| | | GTTVTVSS |
| mAb- | 22 | EVQLVESGGGLVQPKGSLKLSCAASGFTFNTYAMHWVR |
| 2 | | QAPGKGLEWVARIRSKTYNYATFYADSVKDRFTISRDD |
| | | SQSMLYLQMNNLKTEDTAMFYCVTGSSYAMDYWGQGT |
| | | SVTVSS |
| mAb- | 23 | QVQLQQPGAELVRPGTSVKLSCKASGYTFTSYWMHWIK |
| 4 | | QRPGQGLEWIGVIDPSNSYSNYNQKFKGRATLTVDTYSS |
| | | TAYMQLSSLTSEDSAVYYCARPYNHGRDAMDYWGQGT |
| | | SVTVSS |
| mAb- | 24 | QVQLQQSGAELARPGASVQLSCKASGYTFTSYGISWLKQ |
| 8 | | RAGQGLEWIGEIYPRSDNTYYNEKFKGKATLTADKSSST |
| | | AYMELRSLTSEDSAVYFCARRAGMWFAYWGQGTLVTV |
| | | SA |
| mAb- | 25 | DVQLQESGPGLVKPSQSLSLTCTVTGYSITSDFAWNWTRQ |
| 21 | | FPGNKLEWVGYITYSGTTSYNPSLKSRISITRDTSKNQFF |
| | | LQLSSVTTEDTATYYCARSFHFFAYGFAYWGQGTLVTV |
| | | SA |
| mAb- | 26 | QVQLQQSGAELARPGASVKVSCKASGYTFTSYGISWVK |
| 28 | | QRTGQGLEWIGEIYPTRDYTYYNENFKGKATLTADKSS |
| | | TTAYMELRSLTSEDSAVYFCARDGPPFAYWGQGTLVTV |
| | | SA |
| mAb- | 27 | EVQLQQSGPELVKPGASVKISCKASGYTFTDYYMSWVK |
| 30 | | QSHGRSLEWIGEINPDNGDTSHNQKFKGKATMTVDKSS |
| | | GTGNMEIRSLTSEDAGIYYCTSYFSYWGQGTLVTVSA |
| mAb- | 28 | EVLLQQSVADLVRPGASVKLSCTASGLNIKNTYIHWVKQ |
| 33 | | MPEQGLEWIGRIGPANGNTKYAPKFQGKATITADTSSD |
| | | TAYLQLSSLTSEDTAIYYCALAYYRIYWGQGTTLTVSS |
| mAb- | 29 | EVQLQQSVAELVRPGASVKLSCTASGFNIKNTYMHWVK |
| 35 | | QRPEQGLEWIGRIAPANGNTKYAPKFQDKATITANTSSN |
| | | TAYLQLGSLTSEDTAIYYCAFIYQLPYWGQGTTLTVSS |
| mAb- | 30 | QVQVQQSGPELVKPGTSVKISCKTSGYTFTDYYITWVKQ |
| 36 | | RPGQGLEWIGWIFPGRGISFYNENFKDKATLTVDKSSNT |
| | | AFMLLSSLTSEDSAVYFCARWGGTWFAYWGQGTLITVS |
| | | A |
| mAb- | 31 | EVQLQQSVAELVRPGASVKLSCTASGFNIKNTYMHWVK |
| 37 | | QRPEQGLEWIGRIAPANGNTKYDPKFQDKATITADSSSN |
| | | TAYLHLSSLTSEDTAIYYCAFIYQLPYWGQGSTLTVSS |
| mAb- | 32 | QIQLVQSGPELKKPGETVKISCKASGYTFTSYGVTWVKQ |
| 39 | | APGKGLKWMGWINTYSGVPTYADDFKGRFAFSLETSAS |
| | | TAYLQINNLKNEDTATYFCARSHSNYDFWGQGTTLTVSS |

TABLE 9
Polypeptide sequences of the anti-EpCAM light
chain binding domains. CDRs are
underlined and bolded.

| | SEQ | |
|---|---|---|
| | ID | |
| Name | NO | Sequence |
| mAb-1 | 33 | DIQMTQSPSSLSASLGERVSLTCRASQDIGNSLNWLQQKP |
| | | DGTIKRLIYVTSRLDSGVPKRFSGSRSGSDYFFTISSLES |
| | | EDFADYYCLQYASSPFTFGSGTKLEIK |
| mAb-2 | 34 | DILLTQSPAILSVSPGERVSFSCRASQSIGTSLHWYQQRT |
| | | NGSPRLLIKYASESISGIPSRFSGSGSGTDFTLSINSVES |
| | | AEDIDYYCQQSNSWPYTFGGGTKLEIK |
| mAb-4 | 35 | DVLLTQTPLSLSVSLGDQASISCRSSQSFVHINGNTYLEW |
| | | FLQKPGLSPKLLIYKVSNRFSGVPDRFSGSGSGTDFTLKIS |
| | | RVEAEDLGIYYCFQGSHVPLTFGAGTKLELK |
| mAb-8 | 36 | EIVLTQSPAITAASLGQKVTITCSASSSVSDMHWYQQKSG |
| | | TSPKPWIYEISKLASGVPTRFSGSGSGTSYSLTISSMEAED |
| | | AAIYYCQHWSYPLMTFGGGTKLEIK |
| mAb- | 37 | DVVMTQTPLTLSVTIGQPASISCKSSQSLLDSDGKTYLN |
| 21 | | WLLQRPGQSPKRLIYLVSKLDSGVPDRFTGSGSGTDFTLK |
| | | ISRVEAEDLGVYYCWQGTHFPQTFGGGTKLEIK |
| mAb- | 38 | DIVMTQAAFSNPVTLGTSASISCRSSKSLLHSNGFTYLH |
| 28 | | WYLQKPGQSPQLLIYQMSSLASGVPDRFSSSGSGTDFTLR |
| | | ISRVEAEDVGVYYCAQNLELPRTFGGGTKLEIK |
| mAb- | 39 | DVLMTQSPLSLPVSLGDQASISCRSSQSIVDSDGSTYLEW |
| 30 | | YLQKPGQSPKLLIYKVSNRFSGVPDRFSGSGSGTDFTLKIS |
| | | GVEAEDLGVYYCFQGSHVPYTFGGGTKLEIK |
| mAb- | 40 | DIVLTQSPASLAVSLGQRATISCRASESVDNSGISFMHWY |
| 33 | | QQKPGQSPKLLIYRASNLESGIPARFSGSGSRTDFTLTINP |
| | | VETDDVATYYCQQSYENPWTFGGGTKLEIK |
| mAb- | 41 | DIVLTQSPASLAVSLGQRATISCRASESVDNYGISFMHWF |
| 35 | | QQKPGQPPKLLIFRASSLESGIPARFSGSGSRTDFTLTIN |
| | | PVETDDVATYYCQQSYKDPWTFGGATKLEIK |
| mAb- | 42 | EIVLTQSPAITAAFLGQKVTITCSASSSVSYIHWYQQKSDT |
| 36 | | SPKPWIYEISKLASGVPARFSGSGSGTSYSLTISSMEAEDA |
| | | AIYYCQQWNYPLITFGGGTKLEIK |
| mAb- | 43 | DIVLTQSPTSLAVSLGQRATISCRASESVDNYGISFMHWY |
| 37 | | QQKPGQPPKLLIYRASSLESGIPARFSGSGSRTDFTLTINP |
| | | VESDDVATYYCQQSYKDPWTFGGATKLEIK |
| mAb- | 44 | DIVMTQSQKFMSTTVGDRVSITCQASQNVGTDVGWYQQ |
| 39 | | KPGQSPKLLIYSASNRYTGVPDRFTGSGSGTDFTLTISNM |
| | | QSEDLADYFCQQYSSYPRTFGGGTKLEIK |

TABLE 10
CDR sequences anti-EpCAM heavy
chain binding domains.

| | SEQ | | SEQ | | SEQ | |
|---|---|---|---|---|---|---|
| Anti- | ID | | ID | | ID | |
| body | NO | CDR1 | NO | CDR2 | NO | CDR3 |
| mAb-1 | 45 | SYDIN | 46 | WIGWIYP | 47 | DPYYYGT |
| | | | | RDGSTKY | | IYGYFDV |
| | | | | NERFKG | | |
| mAb-2 | 48 | TYAMH | 49 | RIRSKTY | 50 | GSSYAMD |
| | | | | NYATFYA | | Y |
| | | | | DSVKD | | |
| mAb-4 | 51 | SYWMH | 52 | VIDPSNS | 53 | PYNHGRD |
| | | | | YSNYNQK | | AMDY |
| | | | | FKG | | |
| mAb-8 | 54 | SYGIS | 55 | EIYPRSD | 56 | RAGMWFA |
| | | | | NTYYNEK | | Y |
| | | | | FKG | | |
| mAb-21 | 57 | SDFAWN | 58 | YITYSGT | 59 | SFHFFAY |
| | | | | TSYNPSL | | GFAY |
| | | | | KS | | |
| mAb-28 | 60 | SYGIS | 61 | EIYPTRD | 62 | DGPPFAY |
| | | | | YTYYNEN | | |
| | | | | FKG | | |
| mAb-30 | 63 | DYYMS | 64 | EINPDNG | 65 | YFSY |
| | | | | DTSHNQK | | |
| | | | | FKG | | |
| mAb-33 | 66 | NTYIH | 67 | RIGPANG | 68 | AYYRIY |
| | | | | NTKYAPK | | |
| | | | | FQG | | |
| mAb-35 | 69 | NTYMH | 70 | RIAPANG | 71 | IYQLPY |
| | | | | NTKYAPK | | |
| | | | | FQD | | |
| mAb-36 | 72 | DYYIT | 73 | WIFPGRG | 74 | WGGTWFA |
| | | | | ISFYNEN | | Y |
| | | | | FKD | | |
| mAb-37 | 75 | NTYMH | 76 | RIAPANG | 77 | IYQLPY |
| | | | | NTKYDPK | | |
| | | | | FQD | | |
| mAb-39 | 78 | SYGVT | 79 | WINTYSG | 80 | SHSNYDF |
| | | | | VPTYADD | | |
| | | | | FKG | | |

TABLE 11
CDR sequences anti-EpCAM light
chain binding domains.

| | SEQ | | SEQ | | SEQ | |
|---|---|---|---|---|---|---|
| | ID | | ID | | ID | |
| Antibody | NO | CDR1 | NO | CDR2 | NO | CDR3 |
| mAb-1 | 81 | RASQDIG | 82 | VTSRLDS | 83 | LQYASSP |
| | | NSLN | | | | FT |
| mAb-2 | 84 | RASQSIG | 85 | YASESIS | 86 | QQSNSWP |
| | | TSLH | | | | YT |
| mAb-4 | 87 | RSSQSFV | 88 | KVSNRFS | 89 | FQGSH |
| | | HINGNTY | | | | |
| | | LE | | | | |
| mAb-8 | 90 | SASSSVS | 91 | EISKLAS | 92 | QHWSYPL |
| | | DMH | | | | MT |
| mAb-21 | 93 | KSSQSLL | 94 | LVSKLDS | 95 | WQGTH |
| | | DSDGKTY | | | | |
| | | LN | | | | |
| mAb-28 | 96 | RSSKSLL | 97 | QMSSLAS | 98 | AQNLELP |
| | | HSNGFTY | | | | RT |
| | | LH | | | | |
| mAb-30 | 99 | RSSQSIV | 100 | KVSNRFS | 101 | FQGSH |
| | | DSDGSTY | | | | |
| | | LE | | | | |
| mAb-33 | 102 | RASES V | 103 | RASNLES | 104 | QQSYENP |
| | | DNSGISF | | | | WT |
| | | MH | | | | |
| mAb-35 | 105 | RASESVD | 106 | RASSLES | 107 | QQSYKD |
| | | NYGISFM | | | | |
| | | H | | | | |
| mAb-36 | 108 | SASSSVS | 109 | EISKLAS | 110 | QQWNYPL |
| | | YIH | | | | IT |
| mAb-37 | 111 | RASES V | 112 | RASSLES | 113 | QQSYKD |
| | | DNYGISF | | | | |
| | | MH | | | | |
| mAb-39 | 114 | QASQNVG | 115 | SASNRYT | 116 | QQYSSYP |
| | | TDVG | | | | RT |

### 5.6.2 Binding Affinities of Anti-EpCAM Binding Domains

The chimeric anti-EpCAM antibodies bind to human-EpCAM with EC50 values ranging from 0.1 nM to 1 nM by ELISA.

Binding of the chimeric antibodies to cell-surface EpCAM was tested by flow cytometry with CHO-K1 cells transfected with human EpCAM. mAb-1, mAb-2, mAb-8, mAb-8, mAb-28, mAb-30, mAb-33, mAb-35, mAb-36, mAb-39 and EpCAM BMK-6 bound to CHO-K1-huEpCAM cells with EC50s between from 0.5 nM and 10 nM.

Selected chimeric antibodies were also tested for their ability to bind to the surface of tumor cell lines that express EpCAM. In a flow cytometry assay, mAb-1, mAb-2, mAb-8, mAb-21, and EpCAM BMK-6 bound to MCF-7, Caco-2, and A431 cells with EC50s of less than 20 nM.

Binding affinities of the chimeric antibodies to the extracellular domain (ECD) of human EpCAM (Novoprotein #C339) were measured by surface plasmon resonance. In this assay, mAb-1, mAb-2, mAb-4, mAb-8 and EpCAM BMK-6 had binding affinities ranging from 2 nM to 10 nM and mAb-28, mAb33, and mAb35 had binding affinities ranging from 0.2 nM to 2 nM.

### 5.7 EXAMPLE 7. BISPECIFIC CD47×EPCAM ANTIBODIES WITH ENGINEERED BINDING DOMAINS

### 5.7.1 Bispecific Antibody Structures

Bispecific antibodies in a three-chain knob into hole format were constructed with an engineered scFv CD47 binding domain derived from VIR47.V8 and an EpCAM binding domain comprising Mab-1, Mab-2 or Mab-8. The sequences of the heavy chain polypeptides of these antibodies are shown in Table 12 and the polypeptide composition of the 3C-KIH EpCAM×CD47 scFv bispecific antibodies are shown in Table 13.

TABLE 12
Heavy chain polypeptide sequences of 3C-KIH
EpCAM-CD47 scFv bispecific antibodies.

| | SEQ | |
|---|---|---|
| | ID | |
| Name | NO | Sequence |
| EpCAM Mab1 | 117 | QVQLQQSGPELVKPGTSVKLSCRAS |
| IgG1 HC | | GYTFTSYDINWVKQRPGQGLEWIGW |
| knob flag | | IYPRDGSTKYNERFKGKATLTVDTS |
| | | SSTAYMEFHTLTSEDSAVYFCARDP |
| | | YYYGTIYGYFDVWGTGTTVTVSSAS |
| | | TKGPSVFPLAPSSKSTSGGTAALGC |
| | | LVKDYFPEPVTVSWNSGALTSGVHT |
| | | FPAVLQSSGLYSLSSVVTVPSSSLG |
| | | TQTYICNVNHKPSNTKVDKKVEPKS |
| | | CDKTHTCPPCPAPELLGGPSVFLFP |
| | | PKPKDTLMISRTPEVTCVVVDVSHE |
| | | DPEVKFNWYVDGVEVHNAKTKPREE |
| | | QYNSTYRVVSVLTVLHQDWLNGKEY |
| | | KCKVSNKALPAPIEKTISKAKGQP |
| | | REPQVYTLPPSRDELTKNQVSLWCL |
| | | VKGFYPSDIAVEWESNGQPENNYKT |
| | | TPPVLDSDGSFFLYSKLTVDKSRWQ |
| | | QGNVFSCSVMHEALHNHYTQKSLSL |
| | | SPGGGGSDYKDDDDK |
| EpCAM Mab2 | 118 | EVQLVESGGGLVQPKGSLKLSCAAS |
| IgG1 HC | | GFTFNTYAMHWVRQAPGKGLEWVAR |
| knob flag | | IRSKTYNYATFYADSVKDRFTISRD |
| | | DSQSMLYLQMNNLKTEDTAMFYCVT |
| | | GSSYAMDYWGQGTSVTVSSASTKGP |
| | | SVFPLAPSSKSTSGGTAALGCLVKD |
| | | YFPEPVTVSWNSGALTSGVHTFPAV |
| | | LQSSGLYSLSSVVTVPSSSLGTQTY |
| | | ICNVNHKPSNTKVDKKVEPKSCDKT |
| | | HTCPPCPAPELLGGPSVFLFPPKPK |
| | | DTLMISRTPEVTCVVVDVSHEDPEV |
| | | KFNWYVDGVEVHNAKTKPREEQYNS |
| | | TYRVVSVLTVLHQDWLNGKEYKCT< |
| | | VSNKALPAPIEKTISKAKGQPREPQ |
| | | VYTLPPSRDELTKNQVSLWCLVKGF |
| | | YPSDIAVEWESNGQPENNYKTTPPV |
| | | LDSDGSFFLYSKLTVDKSRWQQGNV |
| | | FSCSVMHEALHNHYTQKSLSLSPGG |
| | | GGSDYKDDDDK |
| EpCAM Mab8 | 119 | QVQLQQSGAELARPGASVQLSCKAS |
| IgG1 HC Knob | | GYTFTSYGISWLKQRAGQGLEWIGE |
| Flag-tag | | IYPRSDNTYYNEKFKGKATLTADKS |
| | | SSTAYMELRSLTSEDSAVYFCARRA |
| | | GMWFAYWGQGTLVTVSAASTKGPSV |
| | | FPLAPSSKSTSGGTAALGCLVKDYF |
| | | PEPVTVSWNSGALTSGVHTFPAVLQ |
| | | SSGLYSLSSWTVPSSSLGTQTYICN |
| | | VNHKPSNTKVDKKVEPKSCDKTHTC |
| | | PPCPAPELLGGPSVFLFPPKPKDTL |
| | | MISRTPEVTCVVVDVSHEDPEVKFN |
| | | WYVDGVEVHNAKTKPREEQYNSTYR |
| | | VVSVLTVLHQDWLNGKEYKCKVSNK |
| | | ALPAPIEKTISKAKGQPREPQVYTL |
| | | PPSRDELTKNQVSLWCLVKGFYPSD |
| | | IAVEWESNGQPENNYKTTPPVLDSD |
| | | GSFFLYSKLTVDKSRWQQGNVFSCS |
| | | VMHEALHNHYTQKSLSLSPGGGGSD |
| | | YKDDDDK |
| CD47 VIR47.V8 | 120 | EVQLLESGGGLVQPGGSLRLSCAAS |
| scFv | | GFTFSNFAMSWVRQAPGKGLEWVST |
| IgG1 Hole | | ISASGGRTFYADSVKGRFTISRDNS |
| His-tag | | KNTLYLQMNSLRAEDTAVYYCAKEG |
| | | SFGEGVDPWGQGTLVTVSSASTKGP |
| | | RGSTSGGGSGGGSGGGGSSSYELTQ |
| | | PPSVSVSPGQTARITCSGDALPKKY |
| | | AYWYQQKSGQAPVLVIYEDNKRPSG |
| | | IPERFSGSSSGTMATLTISGAQVED |
| | | EADYYCYSTDISGNHWVFGGGTKLT |
| | | VLASKTHTCPPCPAPELLGGPSVFL |
| | | FPPKPKDTLMISRTPEVTCVVVDVS |
| | | HEDPEVKFNWYVDGVEVHNAKTKPR |
| | | EEQYNSTYRVVSVLTVLHQDWLNGK |
| | | EYKCKVSNKALPAPIEKTISKAKGQ |
| | | PREPQVYTLPPSRDELTKNQVSLSC |
| | | AVKGFYPSDIAVEWESNGQPENNYK |
| | | TTPPVLDSDGSFFLVSKLTVDKSRW |
| | | QQGNVFSCSVMHEALHNHYTQKSLS |
| | | LSPGGGGSHHHHHHHH |

TABLE 13
Polypeptide composition of 3C-KIH EpCAM
- CD47 scFv bispecific antibodies.

EpCAM Arm

| Bispecific | CD47 Arm | HC | LC |
|---|---|---|---|
| Antibody | SEQ ID NO | SEQ ID NO | SEQ ID NO |
| Bi-1 | 120 | 117 | 33 |
| Bi-2 | 120 | 118 | 34 |
| Bi-8 | 120 | 119 | 36 |

### 5.7.2 Binding Activity

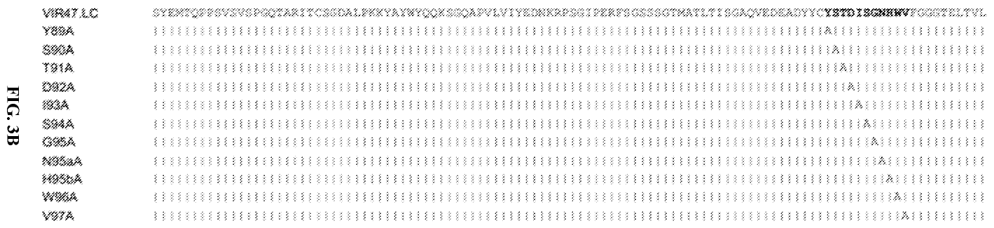

Binding of these bispecific antibodies to human CD47 and human EpCAM was compared by ELISA to the binding of bivalent antibodies with the same binding domains. As expected, the Bi-1, Bi-2, and Bi-8 CD47×EpCAM bispecific antibodies bound to both CD47 and EpCAM. FIG. 4 shows Bi-1 binding to CD47 (FIG. 4A) and EpCAM (FIG. 4B).

### 5.7.3 Inhibition of CD47 Binding to Immobilized SIRPα

Inhibition of CD47 binding to immobilized SIRPα protein was tested by ELISA. The wells of a 96-well plate were coated with 100 μl of 1 μg/ml of CD47 BMK-4 in PBS per well and incubated overnight at 4° C. The following day, the plates were blocked with 300 μl per well of blocking buffer (1% BSA in PBST) for 1.5 hours at 37° C. The plates were then washed three times with PBST. Serial 1:5 dilutions of the mAbs were prepared starting at 30 μg/ml and mixed with huCD47-His (0.11 μg/ml) in a total of 50 μl in blocking buffer and added to the coated plates. After a 1-hour incubation at 37° C., the plates were washed three times with PB ST. 100 μl of THE™ His Antibody [HRP] (Genscript, cat #A00612) diluted 1:5000 was added to each well and followed with 45 minutes of incubation at for 37° C. 100 μl of TMB (InnoReagents, cat #TMB-S-003) was added to each well and developed at room temperature for 5 minutes. Reactions were quenched using 50 μl of 1N HCl. Reaction product on the plates was quantified at 450 nm.

The ability of the CD47×EpCAM bispecific antibodies to inhibit CD47 binding to SIRPα was measured by ELISA and compared with CD47 BMK-1. The Bi-1, Bi-2 and Bi-8 bispecific antibodies inhibited SIRPα binding to CD47 protein. The CD47 BMK-1 antibody, which has two CD47-binding domains, inhibited SIRPα binding to CD47 protein at 5-fold to 50-fold lower concentrations than the bispecific antibodies.

### 5.7.4 Inhibition of SIRPA Binding to CD47+ Cells

Tumor cells were harvested, centrifuged, and then resuspended in FACS buffer (PBS plus 2% FBS) at a concentration of 2×10⁶ cells/mL. 100 μL of the cell suspension was dispensed into each well of a 96-well plate. The plate was centrifuged for 5 min and 300 g, and the supernatants were discarded. The cells were incubated with 50 μl per well of serially-diluted bispecific or bivalent anti-CD47 antibodies and a constant amount of SIRPα-mIgG2a fusion protein (0.2 μg/ml for Raji cells; 2.5 μg/ml for OVISE cells) in FACS buffer for 1 h at 4° C. Then, the plates were washed twice with FACS buffer and incubated for 1 hour at 4° C. in the dark with 100 μL of Alexa Fluor 488 donkey anti-Mouse IgG(H+L) secondary antibody (Invitrogen, Cat #A21202, 1:1000). After washing twice with FACS buffer, the plates were resuspended with 300 μL FACS buffer and analyzed by flow cytometry.

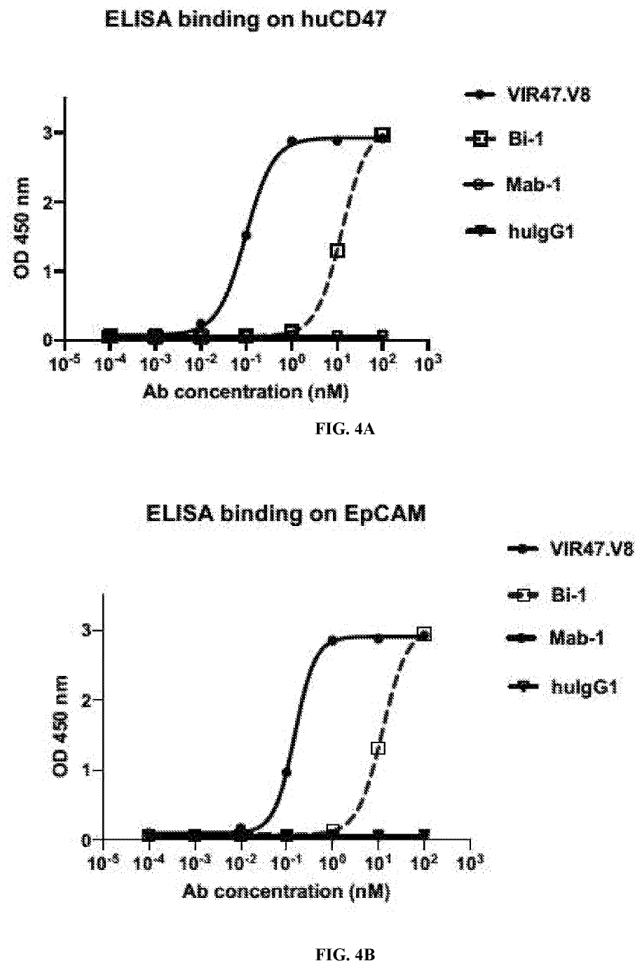

Bispecific antibodies Bi-2 inhibited SIRPα binding to CD47+/EpCAM+ OVISE cells with an EC50 less than the EC50 for αCD47 VIR47.V8, a bivalent antibody with the same CD47-binding domain (FIG. 5A). In contrast, Bi-2 and Bi-8 were much less effective at inhibiting SIRPα binding to Raji cells, which are CD47+ but express >40-fold less EpCAM than OVISE cells (FIG. 5B; see Table 2) These results demonstrate the importance of EpCAM expression for specific targeting of bispecific CD47×EpCAM antibodies to CD47+/EpCAM+ cells and the ability of bispecific CD47×EpCAM antibodies to inhibit the CD47/SIRPα interaction on CD47+/EpCAM+ cells.

### 5.7.5 Phagocytosis of CD47+ EpCAM+ Cancer Cell Lines

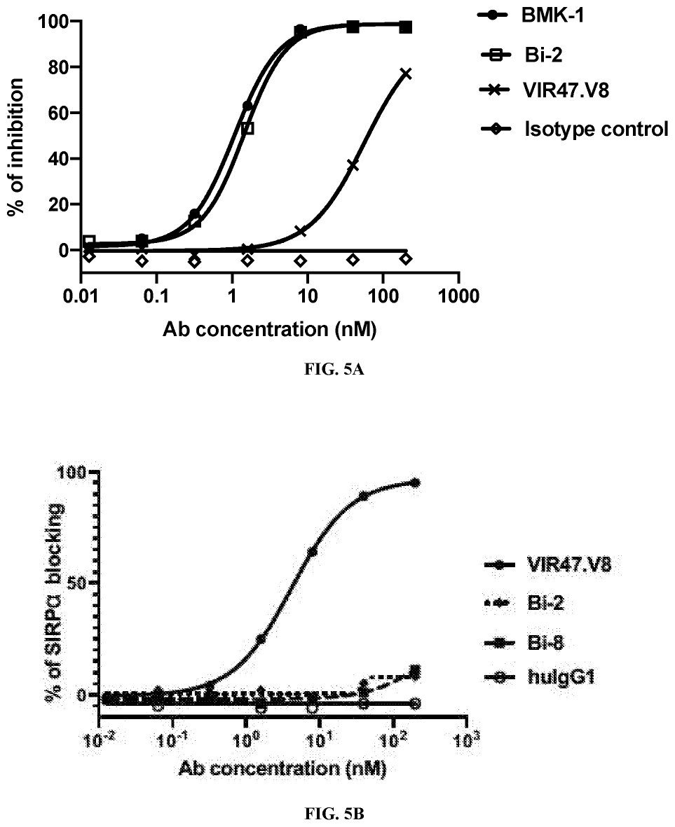

The ability of Bi-1, Bi-2, and Bi-8 to promote phagocytosis of A431 vulvar cancer cells was tested in an ADCP assay and compared with bivalent IgG1 antibodies having the same CD47 or EpCAM binding domains. All three bispecific antibodies promoted phagocytosis of A431 cells. Phagocytosis increased by >10-fold compared to a non-specific IgG1 control. The Bi-1, Bi-2 and Bi-8 bispecific antibodies had lower EC50 values (0.01-3 nM) than a bivalent αCD47 antibody with the same anti-CD47 binding domain (>10 nM). Results of Bi-2 as an example were shown in FIG. 6. The anti-EpCAM mAbs presumably act independently of the CD47-SIRPα don't-eat-me signal and promote phagocytosis through a direct interaction with Fc receptors on phagocytic cells.

### 5.7.6 Reduced Off-Target Binding to Non-Tumor Cells

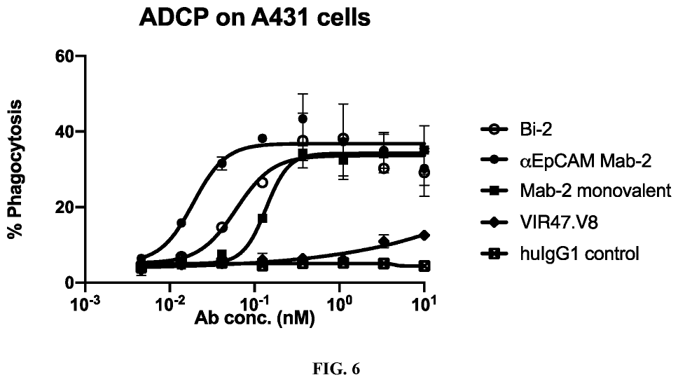

Off-tumor effects of the Bi-1, Bi-2 and Bi-8 CD47×EpCAM bispecific antibodies were tested in hemagglutination and flow cytometry assays. The bivalent and bispecific antibodies with a CD47-binding domain derived from VIR47 did not induce hemagglutination of RBCs, in contrast to the CD47 BMK-1 benchmark antibody, as shown in FIG. 7.

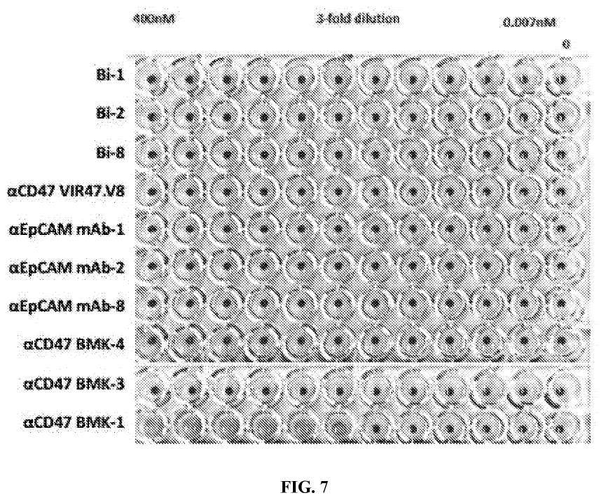

In flow cytometry assays, no binding of Bi-1, Bi-2, or Bi-8 to RBCs was detected at concentrations as high as 500 nM. Significant binding of the CD47 BMK-1 benchmark antibody was detected with an EC50 of approximately 1 nM. The parental bivalent anti-CD47 antibody VIR47.V8 mAb had substantially less binding than CD47 BMK-1 and less binding at lower concentrations than the CD47 BMK-3 benchmark antibody, results of Bi-2 as an example were shown in FIG. 8A.

On platelets, a modest amount of Bi-1, Bi-2, and Bi-8 binding was detected at concentrations of 100 nM and higher, as shown in FIG. 8B. Bi-1, Bi-2 and Bi-8 had less binding to platelets than any of the anti-CD47 benchmark reagents, including the CD47 BMK-1 and CD47 BMK-3 mAbs. Furthermore, parental anti-CD47 VIR47.V8 antibody had lower affinity than both benchmark anti-CD47 antibodies.

### 5.7.7 Antibody-Dependent Cellular Cytotoxicity

CD47×EpCAM bispecific antibodies may also mediate their therapeutic effects by antibody-dependent cellular cytotoxicity (ADCC). ADCC activity was measured using cancer cell lines with various CD47 and EpCAM expression levels, as shown in Table 14. Bi-1, Bi-2, and Bi-8 had ADCC activity in all CD47+ EpCAM+ cell lines tested. Bi-8, which has the highest EpCAM affinity, had the highest ADCC activity. The parental anti-CD47 antibody VIR47.V8 had less ADCC than Bi-8 in all cell lines, and had less ADCC activity than Bi-2, with medium EpCAM affinity, in all cell lines except SKOV-3 ovarian cancer cells, which have relatively higher CD47 expression and lower EpCAM expression.

TABLE 14
Antibody-dependent cellular cytotoxicity activity of
bispecific and bivalent antibodies against cancer cell
lines. Results are presented as EC50 (nM) values.

| | |
|---|---|
| Antibody or | Cell line |

| reagent | A431 | HCC-44 | SKOV-3 | CFPAC-1 |
|---|---|---|---|---|
| Bi-1 | 0.13 | 0.46 | 0.37 | 0.08 |
| Bi-2 | 0.03 | 0.33 | 0.14 | 0.02 |
| Bi-8 | 0.02 | 0.09 | 0.05 | 0.01 |
| αCD47 | 0.28 | 0.51 | 0.08 | 0.11 |
| VIR47.V8 | | | | |
| IgG1 | n.d. | n.d. | n.d. | n.d. |

*n.d.: not determined; N/A: not applicable (not tested)

### 5.8 EXAMPLE 8: GENERATION OF ANTI-CD47/ANTI-EGFR BISPECIFIC ANTIBODIES

Anti-CD47/EGFR bispecific antibodies with different binding stoichiometry and geometry were designed and generated using VH and VL sequences from the anti-CD47 antibody VIR47.V8 and anti-EGFR antibody Cetuximab sequences. The corresponding heavy chain (HC) and light chain (LC) DNAs were synthesized and cloned into the pRK5 mammalian expression vector (ATCC). Each HC and LC pair were then co-transfected in CHO cells. The conditioned medium was harvested by centrifugation (4° C., 4000 rpm for 40 min), then filtered to remove cell debris. The clarified medium was loaded onto MabSelect SuRe column (GE, 17-5438) which was pre-equilibrated with Buffer A (25 mM Tris, 150 mM NaCl, pH 8.0). The column was washed sequentially with 5 column volume of Buffer A, then 30 column volume of Buffer B (Buffer A+0.1% Triton X100+0.1% Triton X114), then 15 column volume of Buffer A. The antibodies were eluted with Buffer C (100 mM sodium citrate, 150 mM NaCl, pH 3.0) and neutralized immediately with Buffer D (200 mM Arginine, 137 mM Succinic acid, pH 5.0). The final product was dialyzed against the buffer PBS, pH 7.4, concentrated and filtrated through MILLEX-MP 0.22 um (MILLIPORE).

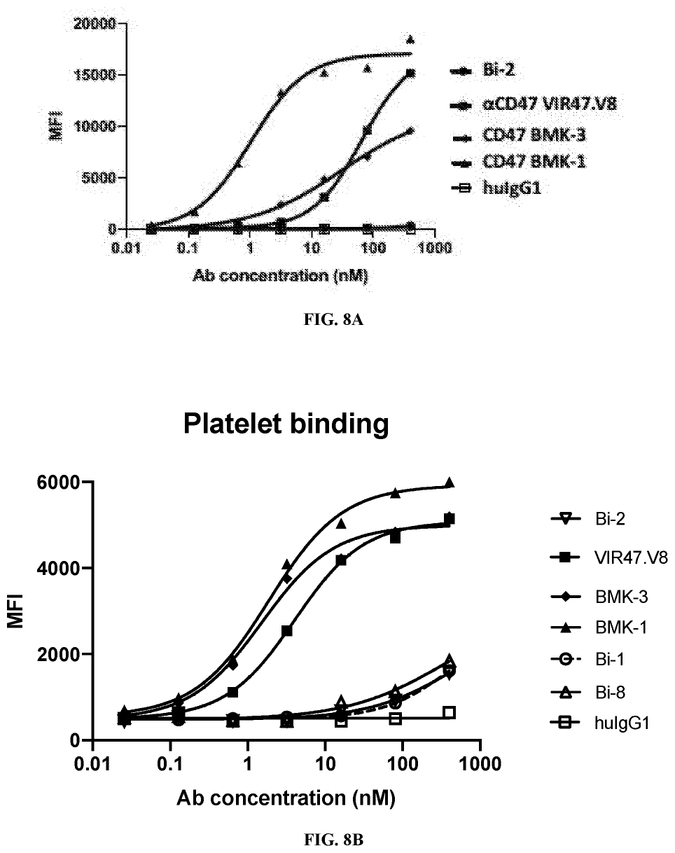

FIGS. 9A-9B illustrates the bispecific formats that were generated for evaluation, and are referred to as Cetuximab.Fab/VIR47.V8.scFv.bispecific (9A) and VIR47.V8.Fab/Cetuximab.scFv.bispecific (9B), and the amino acid and nucleotide sequences are provided in Tables 15-17.

TABLE 15
CDR sequences of Cetuximab (anti-EGFR)
as determined by Kabat definition.

| | SEQ | | SEQ | | SEQ | |
|---|---|---|---|---|---|---|
| | ID | | ID | | ID | |
| CHAIN | NO | CDR1 | NO | CDR2 | NO | CDR3 |
| Heavy | 121 | NYGVH | 122 | VIWSGGNT | 123 | ALTYYD |
| Light | | | | DYNTPFTS | | YEFAY |
| | 124 | RASQSIGTNIH | 125 | YASESIS | 126 | QQNNNW |
| | | | | | | PTT |

TABLE 16
Cetuximab.Fab/VIR47.V8 scFv.bispecific

| | SEQ | |
|---|---|---|
| | ID | |
| Name | NO | Sequence |
| Cetuximab heavy | 127 | QVQLKQSGPGLVQPSQSLSITCTVS |
| chain variable | | GFSLTNYGVHWVRQSPGKGLEWLGV |
| domain | | IWSGGNTDYNTPFTSRLSINKDNSK |
| | | SQVFFKMNSLQSNDTAIYYCARALT |
| | | YYDYEFAYWGQGTLVTVSA |
| Cetuximab light | 128 | DILLTQSPVILSVSPGERVSFSCRA |
| chain variable | | SQSIGTNIHWYQQRTNGSPRLLIKY |
| domain | | ASESISGIPSRFSGSGSGTDFTLSI |
| | | NSVESEDIADYYCQQNNNWPTTFGA |
| | | GTKLELK |
| Cetuximab Knob | 130 | QVQLKQSGPGLVQPSQSLSITCTVS |
| HC | | GFSLTNYGVHWVRQSPGKGLEWLGV |
| | | IWSGGNTDYNTPFTSRLSINKDNSK |
| | | SQVFFKMNSLQSNDTAIYYCARALT |
| | | YYDYEFAYWGQGTLVTVSAASTKGP |
| | | SVFPLAPSSKSTSGGTAALGCLVKD |
| | | YFPEPVTVSWNSGALTSGVHTFPAV |
| | | LQSSGLYSLSSWTVPSSSLGTQTYI |
| | | CNVNHKPSNTKVDKKVEPKSCDKTH |
| | | TCPPCPAPELLGGPSVFLFPPKPKD |
| | | TLMISRTPEVTCVVVDVSHEDPEVK |
| | | FNWYVDGVEVHNAKTKPREEQYNST |
| | | YRVVSVLTVLHQDWLNGKEYKCKVS |
| | | NKALPAPIEKTISKAKGQPREPQVY |
| | | TLPPSRDELTKNQVSLWCLVKGFYP |
| | | SDIAVEWESNGQPENNYKTTPPVLD |
| | | SDGSFFLYSKLTVDKSRWQQGNVFS |
| | | CSVMHEALHNHYTQKSLSLSPGK |
| Cetuximab Knob | 131 | CAGGTGCAGCTGAAGCAGAGCGGCC |
| HC (nucleotide) | | CCGGCCTGGTGCAGCCATCACAGAG |
| | | CCTGAGCATCACATGCACAGTGAGC |
| | | GGATTCAGCCTGACAAACTACGGCG |
| | | TGCACTGGGTGAGACAGAGCCCCGG |
| | | CAAAGGACTGGAGTGGCTGGGAGTG |
| | | ATTTGGTCCGGCGGCAACACCGACT |
| | | ACAACACCCCCTTCACAAGCAGGCT |
| | | GAGCATCAACAAGGACAATAGCAAG |
| | | AGCCAGGTGTTCTTCAAGATGAATA |
| | | GCCTGCAGTCCAACGATACCGCCAT |
| | | CTACTACTGCGCCAGAGCCCTGACC |
| | | TACTACGACTACGAATTCGCCTACT |
| | | GGGGCCAGGGAACCCTGGTGACCGT |
| | | GAGCGCCGCTAGCACCAAAGGTCCC |
| | | AGCGTGTTCCCACTCGCCCCGAGTT |
| | | CAAAATCAACTTCTGGAGGCACCGC |
| | | CGCCCTGGGTTGCCTGGTAAAGGAC |
| | | TACTTCCCAGAGCCCGTGACCGTGA |
| | | GCTGGAACTCCGGGGCACTGACATC |
| | | TGGCGTTCATACTTTCCCGGCCGTG |
| | | CTCCAGTCTTCAGGTCTGTATAGTC |
| | | TCTCCTCTGTGGTCACTGTCCCATC |
| | | TAGCTCTCTGGGCACCCAAACCTAC |
| | | ATATGCAACGTTAATCACAAGCCGA |
| | | GCAATACTAAAGTTGACAAAAAGGT |
| | | GGAACCCAAGTCTTGTGACAAGACC |
| | | CACACGTGTCCCCCCTGCCCGGCTC |
| | | CTGAGCTGCTTGGCGGCCCCAGCGT |
| | | CTTTCTCTTTCCCCCAAAGCCAAAA |
| | | GATACCTTGATGATCAGCAGAACTC |
| | | CCGAGGTGACATGCGTCGTCGTGGA |
| | | CGTAAGCCATGAAGATCCCGAGGTT |
| | | AAGTTCAACTGGTATGTCGATGGCG |
| | | TGGAAGTCCATAATGCTAAGACTAA |
| | | ACCTCGCGAAGAGCAGTACAATTCA |
| | | ACTTACCGGGTCGTTTCCGTTCTGA |
| | | CCGTGCTGCATCAGGACTGGCTGAA |
| | | TGGTAAAGAGTACAAGTGCAAAGTG |
| | | TCTAACAAGGCACTCCCCGCCCCAA |
| | | TTGAGAAGACTATCTCCAAAGCTAA |
| | | AGGGCAACCAAGAGAGCCCCAGGTC |
| | | TACACCCTGCCCCCCTCAAGGGATG |
| | | AGCTTACTAAGAACCAGGTTAGTCT |
| | | CTGGTGCTTGGTTAAAGGATTTTAT |
| | | CCAAGCGATATTGCTGTGGAGTGGG |
| | | AGTCCAACGGCCAGCCTGAGAACAA |
| | | TTATAAAACCACCCCCCCTGTTCTT |
| | | GACAGTGACGGTAGTTTCTTCCTGT |
| | | ATTCCAAACTGACCGTCGATAAGAG |
| | | CAGATGGCAACAGGGAAATGTGTTC |
| | | AGCTGCTCCGTGATGCATGAGGCGC |
| | | TCCATAATCATTACACACAAAAAAG |
| | | TTTGTCCCTGAGCCCAGGCAAG |
| Cetuximab LC | 132 | DILLTQSPVILSVSPGERVSFSCRA |
| | | SQSIGTNIHWYQQRTNGSPRLLIKY |
| | | ASESISGIPSRFSGSGSGTDFTLSI |
| | | NSVESEDIADYYCQQNNNWPTTFGA |
| | | GTKLELKRTVAAPSVFIFPPSDEQL |
| | | KSGTASVVCLLNNFYPREAKVQWKV |
| | | DNALQSGNSQESVTEQDSKDSTYSL |
| | | SSTLTLSKADYEKHKVYACEVTHQG |
| | | LSSPVTKSFNRGEC |
| Cetuximab LC | 133 | GACATATTGTTGACGCAAAGCCCGG |
| (nucleotide) | | TTATACTTAGTGTTAGTCCGGGTGA |
| | | AAGAGTATCTTTTTCTTGTCGCGCT |
| | | TCTCAGTCTATCGGAACAAACATTC |
| | | ATTGGTATCAACAACGGACAAACGG |
| | | AAGCCCTAGACTTCTCATAAAATAC |
| | | GCCTCAGAGTCCATCAGTGGCATAC |
| | | CTTCTCGATTTTCAGGCTCTGGGTC |
| | | TGGTACTGACTTCACATTGTCTATC |
| | | AATTCCGTCGAATCTGAGGACATAG |
| | | CGGACTATTATTGCCAGCAGAATAA |
| | | TAACTGGCCAACTACTTTTGGTGCT |
| | | GGAACTAAGTTGGAGCTTAAGCGTA |
| | | CGGTGGCAGCTCCATCAGTTTTTAT |
| | | CTTCCCACCAAGCGACGAGCAATTG |
| | | AAGTCCGGCACTGCCTCTGTGGTGT |
| | | GCCTTCTGAACAACTTCTATCCAAG |
| | | GGAGGCCAAGGTCCAGTGGAAGGTC |
| | | GATAATGCGCTGCAGAGCGGGAACA |
| | | GCCAAGAGTCAGTGACCGAGCAGGA |
| | | CTCAAAAGATAGCACATACTCTCTG |
| | | AGTTCCACCCTGACCCTGTCAAAGG |
| | | CTGACTACGAAAAGCATAAGGTATA |
| | | CGCATGCGAAGTGACCCATCAGGGT |
| | | CTCTCATCTCCCGTAACCAAATCTT |
| | | TTAATAGAGGAGAATGC |
| CD47 VIR47.V8 | 134 | EVQLLESGGGLVQPGGSLRLSCAAS |
| scFv.hole | | GFTFSNFAMSWVRQAPGKGLEWVST |
| | | ISASGGRTFYADSVKGRFTISRDNS |
| | | KNTLYLQMNSLRAEDTAVYYCAKEG |
| | | SFGEGVDPWGQGTLVTVSSASTKGP |
| | | RGSTSGGGSGGGSGGGGSSSYELTQ |
| | | PPSVSVSPGQTARITCSGDALPKKY |
| | | AYWYQQKSGQAPVLVIYEDNKRPSG |
| | | IPERFSGSSSGTMATLTISGAQVED |
| | | EADYYCYSTDISGNHWVFGGGTKLT |
| | | VLGGSGGSGGASKTHTCPPCPAPEL |
| | | LGGPSVFLFPPKPKDTLMISRTPEV |
| | | TCVVVDVSHEDPEVKFNWYVDGVEV |
| | | HNAKTKPREEQYNSTYRVVSVLTVL |
| | | HQDWLNGKEYKCKVSNKALPAPIEK |
| | | TISKAKGQPREPQVYTLPPSRDELT |
| | | KNQVSLSCAVKGFYPSDIAVEWESN |
| | | GQPENNYKTTPPVLDSDGSFFLVSK |
| | | LTVDKSRWQQGNVFSCSVMHEALHN |
| | | HYTQKSLSLSPGK |
| CD47 VIR47.V8 | 135 | GAGGTACAACTTCTGGAGTCAGGCG |
| scFv.hole | | GGGGGCTAGTCCAACCTGGGGGCTC |
| (nucleotide) | | GCTACGGTTATCTTGCGCCGCGTCT |
| | | GGTTTTACCTTCAGTAACTTTGCCA |
| | | TGTCATGGGTTAGACAAGCACCTGG |
| | | CAAGGGCCTCGAGTGGGTATCGACT |
| | | ATTTCCGCCAGCGGAGGTCGAACAT |
| | | TTTACGCAGACTCGGTGAAAGGTCG |
| | | CTTCACAATTTCTAGGGATAATTCT |
| | | AAAAACACCCTGTATCTTCAAATGA |
| | | ACAGCCTGCGTGCCGAGGACACCGC |
| | | AGTTTACTACTGCGCGAAAGAAGGT |
| | | TCATTTGGTGAAGGCGTCGATCCAT |
| | | GGGGTCAGGGGACCTTAGTTACAGT |
| | | TTCGTCCGCGAGCACGAAGGGTCCT |
| | | AGGGGTTCGACCAGCGGAGGAGGTA |
| | | GTGGGGGAGGCTCAGGTGGGGGAGG |
| | | GTCCAGCAGCTACGAGCTAACCCAA |
| | | CCACCATCAGTATCCGTGTCTCCGG |
| | | GCCAAACGGCGAGAATCACTTGCAG |
| | | CGGAGACGCACTACCGAAAAAGTAT |
| | | GCTTATTGGTACCAACAAAAAAGCG |
| | | GACAGGCGCCGGTTCTCGTCATATA |
| | | TGAAGACAACAAGCGCCCTTCTGGT |
| | | ATTCCGGAGAGGTTCTCCGGGTCGT |
| | | CATCAGGAACAATGGCGACGCTTAC |
| | | CATATCGGGAGCACAAGTAGAAGAT |
| | | GAAGCGGACTACTACTGCTATTCTA |
| | | CTGACATCTCTGGAAACCACTGGGT |
| | | CTTCGGAGGTGGTACCAAGCTTACC |
| | | GTCCTTGGTGGCAGTGGTGGCTCTG |
| | | GGGGCGCTAGCAAGACCCACACGTG |
| | | TCCCCCCTGCCCGGCTCCTGAGCTG |
| | | CTTGGCGGCCCCAGCGTCTTTCTCT |
| | | TTCCCCCAAAGCCAAAAGATACCTT |
| | | GATGATCAGCAGAACTCCCGAGGTG |
| | | ACATGCGTCGTCGTGGACGTAAGCC |
| | | ATGAAGATCCCGAGGTTAAGTTCAA |
| | | CTGGTATGTCGATGGCGTGGAAGTC |
| | | CATAATGCTAAGACTAAACCTCGCG |
| | | AAGAGCAGTACAATTCAACTTACCG |
| | | GGTCGTTTCCGTTCTGACCGTGCTG |
| | | CATCAGGACTGGCTGAATGGTAAAG |
| | | AGTACAAGTGCAAAGTGTCTAACAA |
| | | GGCACTCCCCGCCCCAATTGAGAAG |
| | | ACTATCTCCAAAGCTAAAGGGCAAC |
| | | CAAGAGAGCCCCAGGTCTACACCCT |
| | | GCCCCCCTCAAGGGATGAGCTTACT |
| | | AAGAACCAGGTTAGTCTCAGCTGCG |
| | | CGGTTAAAGGATTTTATCCAAGCGA |
| | | TATTGCTGTGGAGTGGGAGTCCAAC |
| | | GGCCAGCCTGAGAACAATTATAAAA |
| | | CCACCCCCCCTGTTCTTGACAGTGA |
| | | CGGTAGTTTCTTCCTGGTTTCCAAA |
| | | CTGACCGTCGATAAGAGCAGATGGC |
| | | AACAGGGAAATGTGTTCAGCTGCTC |
| | | CGTGATGCATGAGGCGCTCCATAAT |
| | | CATTACACACAAAAAAGTTTGTCCC |
| | | TGAGCCCAGGCAAG |

TABLE 17
VIR47.V8.Fab/Cetuximab.scFv.bispecific

| | SEQ | |
|---|---|---|
| | ID | |
| Name | NO | Sequence |
| CD47 VIR47.V8 | 136 | EVQLLESGGGLVQPGGSLRLSCAAS |
| Knob HC.Flag | | GFTFSNFAMSWVRQAPGKGLEWVST |
| | | ISASGGRTFYADSVKGRFTISRDNS |
| | | KNTLYLQMNSLRAEDTAVYYCAKEG |
| | | SFGEGVDPWGQGTLVTVSSASTKGP |
| | | SVFPLAPSSKSTSGGTAALGCLVKD |
| | | YFPEPVTVSWNSGALTSGVHTFPAV |
| | | LQSSGLYSLSSVVTVPSSSLGTQTY |
| | | ICNVNHKPSNTKVDKKVEPKSCDKT |
| | | HTCPPCPAPELLGGPSVFLFPPKPK |
| | | DTLMISRTPEVTCVVVDVSHEDPEV |
| | | KFNWYVDGVEVHNAKTKPREEQYNS |
| | | TYRVVSVLTVLHQDWLNGKEYKCKV |
| | | SNKALPAPIEKTISKAKGQPREPQV |
| | | YTLPPSRDELTKNQVSLWCLVKGFY |
| | | PSDIAVEWESNGQPENNYKTTPPVL |
| | | DSDGSFFLYSKLTVDKSRWQQGNVF |
| | | SCSVMHEALHNHYTQKSLSLSPGGG |
| | | GSDYKDDDDK |
| CD47 VIR47.V8 | 137 | GAGGTGCAGCTGCTGGAGAGCGGGG |
| Knob HC | | GGGGACTGGTGCAGCCAGGAGGAAG |
| (nucleotide) | | CCTGAGACTGAGCTGTGCCGCCAGC |
| | | GGGTTTACATTTAGCAACTTTGCCA |
| | | TGAGCTGGGTGAGACAGGCCCCCGG |
| | | AAAAGGACTGGAGTGGGTGAGCACC |
| | | ATTAGCGCCAGCGGAGGAAGAACAT |
| | | TTTACGCTGATTCCGTGAAGGGGAG |
| | | ATTCACAATCTCCAGAGACAACAGC |
| | | AAGAACACCCTGTACCTGCAGATGA |
| | | ACAGCCTGAGAGCCGAGGACACCGC |
| | | CGTGTATTACTGCGCCAAGGAGGGA |
| | | TCTTTTGGCGAGGGAGTGGACCCCT |
| | | GGGGCCAGGGAACCCTGGTGACCGT |
| | | GAGCAGCGCTAGCACCAAAGGTCCC |
| | | AGCGTGTTCCCACTCGCCCCGAGTT |
| | | CAAAATCAACTTCTGGAGGCACCGC |
| | | CGCCCTGGGTTGCCTGGTAAAGGAC |
| | | TACTTCCCAGAGCCCGTGACCGTGA |
| | | GCTGGAACTCCGGGGCACTGACATC |
| | | TGGCGTTCATACTTTCCCGGCCGTG |
| | | CTCCAGTCTTCAGGTCTGTATAGTC |
| | | TCTCCTCTGTGGTCACTGTCCCATC |
| | | TAGCTCTCTGGGCACCCAAACCTAC |
| | | ATATGCAACGTTAATCACAAGCCGA |
| | | GCAATACTAAAGTTGACAAAAAGGT |
| | | GGAACCCAAGTCTTGTGACAAGACC |
| | | CACACGTGTCCCCCCTGCCCGGCTC |
| | | CTGAGCTGCTTGGCGGCCCCAGCGT |
| | | CTTTCTCTTTCCCCCAAAGCCAAAA |
| | | GATACCTTGATGATCAGCAGAACTC |
| | | CCGAGGTGACATGCGTCGTCGTGGA |
| | | CGTAAGCCATGAAGATCCCGAGGTT |
| | | AAGTTCAACTGGTATGTCGATGGCG |
| | | TGGAAGTCCATAATGCTAAGACTAA |
| | | ACCTCGCGAAGAGCAGTACAATTCA |
| | | ACTTACCGGGTCGTTTCCGTTCTGA |
| | | CCGTGCTGCATCAGGACTGGCTGAA |
| | | TGGTAAAGAGTACAAGTGCAAAGTG |
| | | TCTAACAAGGCACTCCCCGCCCCAA |
| | | TTGAGAAGACTATCTCCAAAGCTAA |
| | | AGGGCAACCAAGAGAGCCCCAGGTC |
| | | TACACCCTGCCCCCCTCAAGGGATG |
| | | AGCTTACTAAGAACCAGGTTAGTCT |
| | | CTGGTGCTTGGTTAAAGGATTTTAT |
| | | CCAAGCGATATTGCTGTGGAGTGGG |
| | | AGTCCAACGGCCAGCCTGAGAACAA |
| | | TTATAAAACCACCCCCCCTGTTCTT |
| | | GACAGTGACGGTAGTTTCTTCCTGT |
| | | ATTCCAAACTGACCGTCGATAAGAG |
| | | CAGATGGCAACAGGGAAATGTGTTC |
| | | AGCTGCTCCGTGATGCATGAGGCGC |
| | | TCCATAATCATTACACACAAAAAAG |
| | | TTTGTCCCTGAGCCCAGGCGGCGGA |
| | | GGCAGCGACTACAAGGACGACGATG |
| | | ATAAG |
| CD47 VIR47.V8 | 14 | SYELTQPPSVSVSPGQTARITCSGD |
| LC | | ALPKKYAYWYQQKSGQAPVLVIYED |
| | | NKRPSGIPERFSGSSSGTMATLTIS |
| | | GAQVEDEADYYCYSTDISGNHWVFG |
| | | GGTKLTVLGQPKAAPSVTLFPPSSE |
| | | ELQANKATLVCLISDFYPGAVTVAW |
| | | KADSSPVKAGVETTTPSKQSNNKYA |
| | | ASSYLSLTPEQWKSHRSYSCQVTHE |
| | | GSTVEKTVAPTECS |
| CD47 VIR47.V8 | 138 | TCTTACGAGCTGACCCAGCCTCCTT |
| LC (nucleotide) | | CTGTGTCTGTGTCTCCCGGTCAGAC |
| | | CGCTAGAATCACCTGCTCTGGCGAC |
| | | GCTCTGCCTAAGAAGTACGCCTACT |
| | | GGTACCAGCAGAAGTCCGGCCAGGC |
| | | TCCTGTGCTGGTGATTTACGAGGAC |
| | | AATAAGAGACCCTCCGGCATCCCCG |
| | | AGAGATTCTCTGGATCCTCCTCCGG |
| | | AACCATGGCCACACTGACTATCTCC |
| | | GGCGCTCAGGTGGAGGATGAAGCTG |
| | | ATTATTACTGCTACTCCACCGACAT |
| | | CTCCGGCAACCACTGGGTGTTCGGA |
| | | GGTGGAACAAAGCTGACCGTGCTGG |
| | | GACAGCCTAAGGCTGCTCCTTCTGT |
| | | GACTCTGTTTCCTCCCAGCTCCGAG |
| | | GAGCTGCAGGCTAACAAAGCTACTC |
| | | TGGTCTGCCTGATCAGTGATTTCTA |
| | | CCCCGGCGCCGTCACCGTGGCTTGG |
| | | AAGGCTGATTCTTCCCCCGTGAAGG |
| | | CCGGTGTGGAAACTACAACACCCTC |
| | | CAAACAGTCCAACAACAAATACGCC |
| | | GCCTCCTCCTACCTGTCCCTGACAC |
| | | CTGAACAGTGGAAGTCTCACAGATC |
| | | CTACTCCTGCCAGGTCACCCACGAA |
| | | GGATCCACAGTCGAGAAGACCGTCG |
| | | CCCCTACAGAGTGTTCTTGA |
| Cetuximab | 139 | QVQLKQSGPGLVQPSQSLSITCTVS |
| scFv.hole.his | | GFSLTNYGVHWVRQSPGKGLEWLGV |
| | | IWSGGNTDYNTPFTSRLSINKDNSK |
| | | SQVFFKMNSLQSNDTAIYYCARALT |
| | | YYDYEFAYWGQGTLVTVSAASTKGP |
| | | RGSTSGGGSGGGSGGGGSSDILLTQ |
| | | SPVILSVSPGERVSFSCRASQSIGT |
| | | NIHWYQQRTNGSPRLLIKYASESIS |
| | | GIPSRFSGSGSGTDFTLSINSVESE |
| | | DIADYYCQQNNNWPTTFGAGTKLEL |
| | | KGGSGGSGGASKTHTCPPCPAPELL |
| | | GGPSVFLFPPKPKDTLMISRTPEVT |
| | | CVVVDVSHEDPEVKFNWYVDGVEVH |
| | | NAKTKPREEQYNSTYRVVSVLTVLH |
| | | QDWLNGKEYKCKVSNKALPAPIEKT |
| | | ISKAKGQPREPQVYTLPPSRDELTK |
| | | NQVSLSCAVKGFYPSDIAVEWESNG |
| | | QPENNYKTTPPVLDSDGSFFLVSKL |
| | | TVDKSRWQQGNVFSCSVMHEALHNH |
| | | YTQKSLSLSPGGGGSHHHHHHHH |
| Cetuximab | 140 | CAGGTGCAGCTGAAGCAGAGCGGCC |
| scFv.hole.his | | CCGGCCTGGTGCAGCCATCACAGAG |
| (nucleotide) | | CCTGAGCATCACATGCACAGTGAGC |
| | | GGATTCAGCCTGACAAACTACGGCG |
| | | TGCACTGGGTGAGACAGAGCCCCGG |
| | | CAAAGGACTGGAGTGGCTGGGAGTG |
| | | ATTTGGTCCGGCGGCAACACCGACT |
| | | ACAACACCCCCTTCACAAGCAGGCT |
| | | GAGCATCAACAAGGACAATAGCAAG |
| | | AGCCAGGTGTTCTTCAAGATGAATA |
| | | GCCTGCAGTCCAACGATACCGCCAT |
| | | CTACTACTGCGCCAGAGCCCTGACC |
| | | TACTACGACTACGAATTCGCCTACT |
| | | GGGGCCAGGGAACCCTGGTGACCGT |
| | | GAGCGCCGCTTCTACCAAAGGACCC |
| | | CGGGGCTCTACCTCCGGCGGCGGAA |
| | | GCGGAGGAGGATCTGGAGGAGGAGG |
| | | AAGTTCTGACATATTGTTGACGCAA |
| | | AGCCCGGTTATACTTAGTGTTAGTC |
| | | CGGGTGAAAGAGTATCTTTTTCTTG |
| | | TCGCGCTTCTCAGTCTATCGGAACA |
| | | AACATTCATTGGTATCAACAACGGA |
| | | CAAACGGAAGCCCTAGACTTCTCAT |
| | | AAAATACGCCTCAGAGTCCATCAGT |
| | | GGCATACCTTCTCGATTTTCAGGCT |
| | | CTGGGTCTGGTACTGACTTCACATT |
| | | GTCTATCAATTCCGTCGAATCTGAG |
| | | GACATAGCGGACTATTATTGCCAGC |
| | | AGAATAATAACTGGCCAACTACTTT |
| | | TGGTGCTGGAACTAAGTTGGAGCTT |
| | | AAGGGTGGCAGTGGTGGCTCTGGGG |
| | | GCGCTAGCAAGACCCACACGTGTCC |
| | | CCCCTGCCCGGCTCCTGAGCTGCTT |
| | | GGCGGCCCCAGCGTCTTTCTCTTTC |
| | | CCCCAAAGCCAAAAGATACCTTGAT |
| | | GATCAGCAGAACTCCCGAGGTGACA |
| | | TGCGTCGTCGTGGACGTAAGCCATG |
| | | AAGATCCCGAGGTTAAGTTCAACTG |
| | | GTATGTCGATGGCGTGGAAGTCCAT |
| | | AATGCTAAGACTAAACCTCGCGAAG |
| | | AGCAGTACAATTCAACTTACCGGGT |
| | | CGTTTCCGTTCTGACCGTGCTGCAT |
| | | CAGGACTGGCTGAATGGTAAAGAGT |
| | | ACAAGTGCAAAGTGTCTAACAAGGC |
| | | ACTCCCCGCCCCAATTGAGAAGACT |
| | | ATCTCCAAAGCTAAAGGGCAACCAA |
| | | GAGAGCCCCAGGTCTACACCCTGCC |
| | | CCCCTCAAGGGATGAGCTTACTAAG |
| | | AACCAGGTTAGTCTCAGCTGCGCGG |
| | | TTAAAGGATTTTATCCAAGCGATAT |
| | | TGCTGTGGAGTGGGAGTCCAACGGC |
| | | CAGCCTGAGAACAATTATAAAACCA |
| | | CCCCCCCTGTTCTTGACAGTGACGG |
| | | TAGTTTCTTCCTGGTTTCCAAACTG |
| | | ACCGTCGATAAGAGCAGATGGCAAC |
| | | AGGGAAATGTGTTCAGCTGCTCCGT |
| | | GATGCATGAGGCGCTCCATAATCAT |
| | | TACACACAAAAAAGTTTGTCCCTGA |
| | | GCCCAGGCGGCGGAGGCAGCCACCA |
| | | CCACCACCACCACCATCAT |

### 5.9 EXAMPLE 9: ANTIBODY BINDING TO ANTIGEN MEASURED BY ELISA

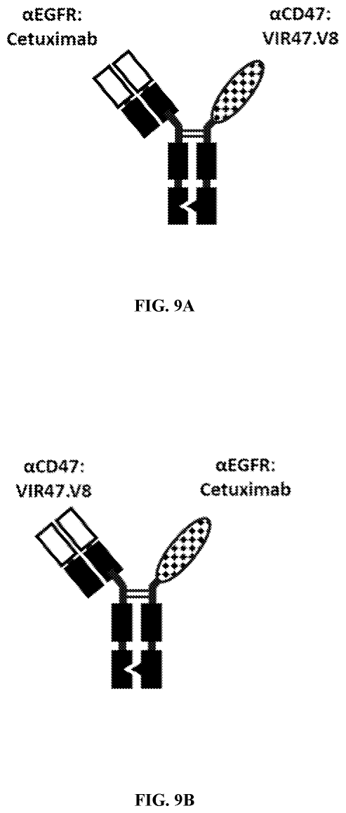

A 96-well plate was coated overnight at 4° C. with 1 μg/ml recombinant huCD47 or huEGFR. After washing 3 times, the plate was blocked with 300 μl 1% BSA in PBST at 37° C. for 1 hour. Serially diluted antibodies were added and incubated at 37° C. for 1 hour. The plate was then washed 4 times with PBST and incubated with 1:5000 diluted peroxidase labeled goat anti-human IgG (Fab specific) secondary antibody (Sigma, Cat #A0293) for 1 hour at 37° C. The plate was washed again 4 times with PBST, incubated with 3,3′,5,5′-tetramethylbenzidine (TMB) substrate for 15 min at room temperature, terminated with 1N HCl, and then read at 450 nm. FIGS. 10A-10B showed the ELISA binding results for anti-CD47/EGFR bispecific antibody on human CD47 and human EGFR, respectively.

### 5.10 EXAMPLE 10: INHIBITION OF SIRPα BINDING TO CD47+ CELLS

Tumor cells were harvested, centrifuged, and then resuspended in FACS buffer (PBS plus 2% FBS) at a concentration of 2×10⁶ cells/mL. 100 μL of the cell suspension was dispensed into each well of a 96-well plate. The plate was centrifuged for 5 min and 300 g, and the supernatants were discarded. The cells were incubated with 50 μl per well of serially diluted bispecific or bivalent anti-CD47 antibodies and a constant amount of SIRPα-mIgG2a fusion protein (1 μg/ml for A431 cells) in FACS buffer for 1 h at 4° C. Then, the plates were washed twice with FACS buffer and incubated for 1 hour at 4° C. in the dark with 100 μL of Alexa Fluor 488 donkey anti-Mouse IgG(H+L) secondary antibody (Invitrogen, Cat #A21202, 1:1000). After washing twice with FACS buffer, the plates were resuspended with 300 μL FACS buffer and analyzed by flow cytometry.

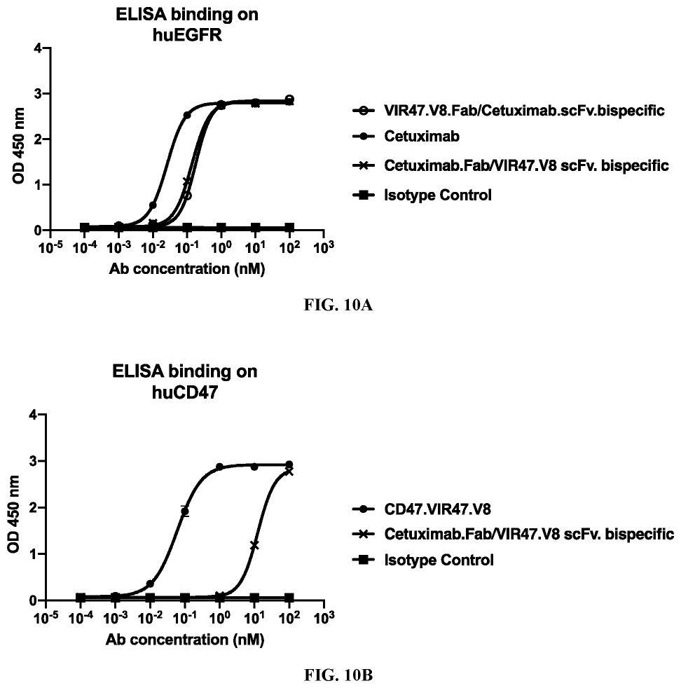

FIG. 11 showed the SIRPα blocking results for anti-CD47/EGFR bispecific antibody on A431 cells which showed high expression of CD47 as well as EGFR, with the guide EGFR, the bispecific VIR47.V8/cetuximab showed superior SIRPα blocking activity than VIR47.V8.

### 5.11 EXAMPLE 11: ANTIBODY-DEPENDENT CELLULAR PHAGOCYTOSIS

The bispecific antibodies were tested in an antibody-dependent cellular phagocytosis (ADCP) assay. Peripheral blood mononuclear cells (PBMCs) were isolated from human donors. Monocytes were enriched using a Human Monocyte Enrichment Kit without CD16 depletion (STEMCELL, Cat #19058). Isolated monocytes were differentiated into macrophages by culturing monocytes in complete culture media (RPMI 1640+10% FBS) with 20 ng/ml of human Macrophage Colony-Stimulating Factor (M-CSF, Peprotech, Cat #: 3-25-10). The media was changed every three days. After 7 days of culturing in M-CSF containing culture media, macrophages were collected and counted. Target tumor cells were collected and washed with D-PBS two times to remove remaining FBS. The washed tumor cells were resuspended in PBS at a cell concentration of 5-10×10⁶/mL. Cancer cells were stained with CFSE (ebiosciences, Cat #: 65-0580-84) at a final concentration of 3 μM and mixed immediately. The cells were stained in the dark at room temperature for 10 minutes. The staining was terminated by adding 4-5 volumes of cold complete media and incubating on ice for 5 minutes. Stained cells were washed three times with RPMI 1640+10% FBS. Cells were resuspended in 1 ml RPMI 1640+10% FBS and counted, then adjusted the cell numbers to 3×10⁵ cells/mL. 50 μl cells were seeded into a 96 well deep U-plate (Axygen, Cat #: P-DW-20-C) wherein each well contained 1.5×104 cells. 50 μl of diluted antibodies were added to each well. 100 μl of macrophages (1.5×104 cells) were added to each well and incubated at 37° C., 5% CO2 for 1.5 hours. After incubation, cells were washed with 2 ml of 2% FBS in D-PBS once. 100 μl of diluted Fc blocker (Human TruStain FcX (Fc Receptor Blocking Solution), Biolegend Cat #: 422302)) was added and the cells were incubated at room temperature for 10 minutes. 20 μl of diluted anti-human CD11b antibody was added to each well and incubated for 30 minutes at 4° C. in the dark. Cells were washed with 2% FBS-D-PBS once. Phagocytosis was detected in a flow cytometer by the appearance of CFSE/CD11b double positive cells indicative of macrophages that engulfed the tumor cells.

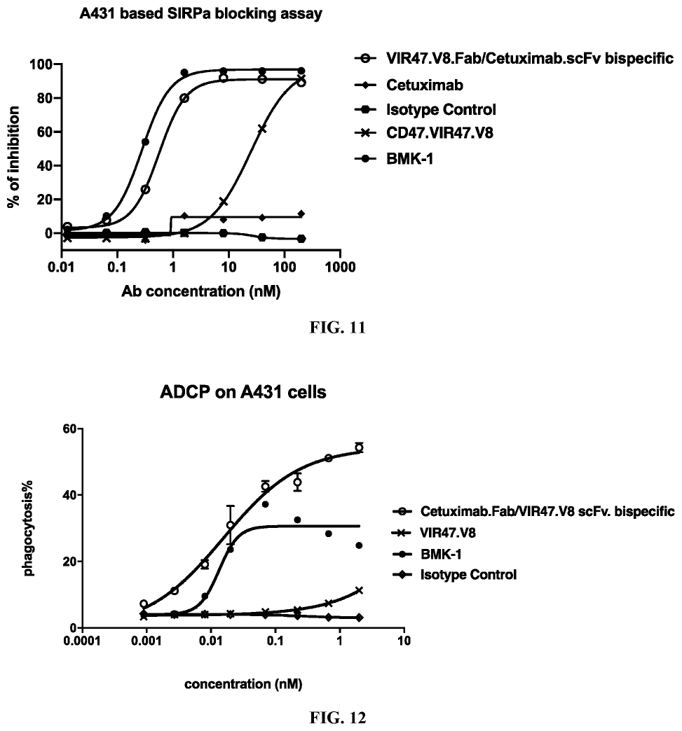

In FIG. 12, Bispecific antibody Cetuximab/VIR47.V8 showed superior ADCP activity as compared with VIR47.V8 bivalent antibody and BMK-1.

### 5.12 EXAMPLE 12: ANTIBODY BINDING TO RED BLOOD CELLS/PLATELET AND HEMAGGLUTINATION

The RBC binding assay was performed by spinning down fresh human whole blood at 200 g for 10 minutes. Collected RBCs were washed twice with PBS and counted using flow cytometry. 1×10⁶ cells were dispensed into each well of a 96 well culture plate. Serially diluted anti-CD47 antibodies were added and incubated for 1 hour at 4° C. Cells were washed with FACS buffer (PBS+2% FBS) twice. Secondary antibody (Alexa Fluor® 488 Goat Anti-Human IgG (H+L)) was added and incubated for 1 hour at 4° C. Cells were washed twice and resuspended in 200 μl of FACS buffer and analyzed by flow cytometry.

The hemagglutination assay was performed by diluting human red blood cells (RBCs) and incubating RBCs at 37° C. for 2 hours with a titration of CD47 antibodies (from 100 μg/ml) in a round bottom 96 well plate. Hemagglutination is demonstrated by the presence of crosslinked RBCs, which appear as a haze because they do not settle to the bottom of the well, in contrast to non-hemagglutinated RBCs

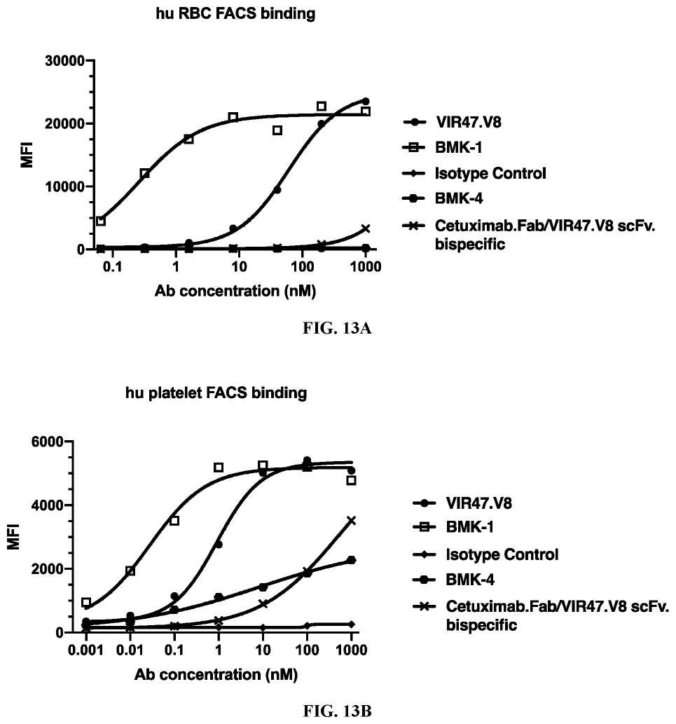

In FIGS. 13A-13B, bispecific antibody Cetuximab/VIR47.V8 showed much less RBC and platelet binding compared to the CD47 BMK-1 benchmark antibody and its parental VIR47.V8 bivalent antibody. Furthermore, hemagglutination was not detected for VIR47.V8/Cetuximab, even at the highest tested concentration (666.7 nM; data was not shown).

### 5.13 EXAMPLE 13: IN VIVO ANTI-TUMOR ACTIVITIES OF CD47×EGFR BISPECIFIC ANTIBODIES WERE INVESTIGATED IN A SNU-5 GASTRIC CANCER XENOGRAFT MODEL

The SNU-5 tumor cells were cultured in Iscove's Modified Dulbecco's medium supplemented with 20% heat-inactivated fetal bovine serum at 37° C. in an atmosphere of 5% CO2 in air. Tumor cells growing in an exponential growth phase were harvested for tumor inoculation. Female CB17/SCID mice of 6-7 weeks in age (Shanghai Lingchang Biotechnology) were inoculated subcutaneously with 10⁷ cells in 0.2 mL of PBS supplemented with Matrigel (1:1) for tumor development. Tumor volume was measured using a caliper device and calculated with the following formula: Tumor volume=(length×width²)/2. On day 6 post tumor inoculation when the mean tumor volume reached approximately 175 mm³, animals were randomized by tumor volumes into 6 animals per group. Test antibodies were administered through bolus tail vein injection at a dose of 10 mg/kg, once per week for five consecutive weeks. Tumor volume was measured twice weekly. Animals were humanely euthanized if their tumor volumes exceeded 3000 mm³ or if they experienced over 20% of body weight loss.

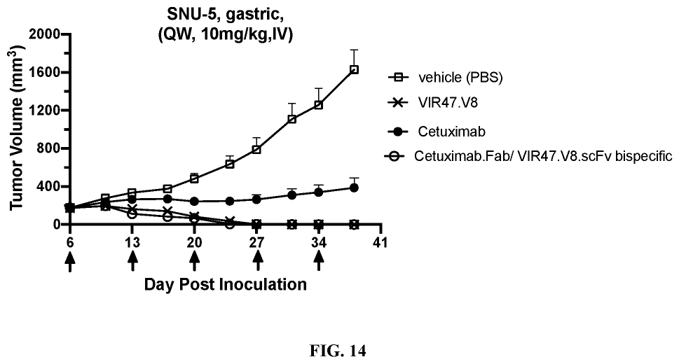

The results are summarized in FIG. 14. Treatment with bispecific antibodies CD47×EGFR, and bivalent CD47 antibody led to complete tumor regression whereas treatment with cetuximab antibody only inhibited tumor growth. Moreover, all mice in CD47×EGFR bispecific antibody groups and the CD47 antibody group remained tumor free until the end of the study. These data demonstrate the superior in vivo anti-tumor activities of CD47×EGFR bispecific antibodies over their monospecific parents cetuximab against EGFR and CD47 expressing gastric cancer.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A bispecific antibody comprising a CD47-binding domain and an EpCAM-binding domain, wherein the bispecific antibody has a higher affinity for CD47 expressed on the surface of a tumor cell than for CD47 expressed on the surface of a red blood cell or a platelet.

2. The bispecific antibody of claim 1, further comprising an Fc domain.

3. The bispecific antibody of claim 1, wherein the tumor cell expresses EpCAM.

4. The bispecific antibody of claim 1, wherein a concentration of the bispecific antibody required for half-maximal binding to a human red blood cell is greater than 500 nM.

5. The bispecific antibody of claim 1, wherein the bispecific antibody binds to human CD47 with a KD of less than 100 nM as determined by surface plasmon resonance.

6. The bispecific antibody of claim 1, wherein the bispecific antibody binds to human CD47 with a KD of between 1 nM and 100 nM, between 1 nM and 50 nM, or between 5 nM and 50 nM as determined by surface plasmon resonance.

7. The bispecific antibody of claim 1, wherein the bispecific antibody binds to EpCAM with a KD of less than 500 nM as determined by surface plasmon resonance.

8. The bispecific antibody of claim 1, wherein the bispecific antibody binds to EpCAM with a KD of between 0.2 nM and 500 nM, between 1 nM and 300 nM, between 5 nM and 200 nM, or between 10 nM and 150 nM as determined by surface plasmon resonance.

9. The bispecific antibody of claim 1, wherein the CD47-binding domain is a human or engineered human CD47-binding domain.

10. The bispecific antibody of claim 1, wherein the CD47-binding domain comprises a heavy chain variable domain and a light chain variable domain.

11. The bispecific antibody of claim 1, wherein the CD47-binding domain comprises an scFv.

12. The bispecific antibody of claim 1, wherein the EpCAM-binding domain comprises a heavy chain variable domain and a light chain variable domain.

13. The bispecific antibody of claim 1, wherein the EpCAM-binding domain comprises an scFv.

14. The bispecific antibody of claim 2, wherein the Fc domain is a human Fc domain.

15. The bispecific antibody of claim 14, wherein the isotype of the human Fc domain is IgG1 or IgG4.

16. The bispecific antibody of claim 15, wherein the Fc domain is a heterodimeric Fc domain, wherein the heterodimeric Fc region comprises a knob chain and a hole chain, forming a knob-into-hole (KiH) structure.

17. The bispecific antibody of claim 16, wherein the knob chain comprises the mutation T366W and the hole chain comprises the mutations T366S, L368A, and Y407V, wherein amino acid position numbering is according to the EU index of Kabat et al.

18. The bispecific antibody of claim 1, wherein the bispecific antibody has an asymmetric three-chain knob-into-hole structure.

19. The bispecific antibody of claim 1, wherein the CD47-binding domain is a Fab and the EpCAM-binding domain is an scFv.

20. The bispecific antibody of claim 1, wherein the CD47-binding domain is an scFv and the EpCAM-binding domain is a Fab.

21. The bispecific antibody of claim 1, wherein the CD47-binding domain comprises three heavy chain (HC) complementarity determining regions (CDRs): HC-CDR1, HC-CDR2, and HC-CDR3; and three light chain (LC) CDRs: LC-CDR1, the LC-CDR2, and the LC-CDR3; wherein HC-CDR1 comprises SEQ ID NO: 15, HC-CDR2 comprises SEQ ID NO: 16, HC-CDR3 comprises a sequence with at least 50% identity to SEQ ID NO: 17, LC-CDR1 comprises SEQ ID NO: 18, LC-CDR2 comprises SEQ ID NO: 19, and LC-CDR3 comprises a sequence with at least 50% identity to SEQ ID NO: 20.

22. The bispecific antibody of claim 21, wherein HC-CDR3 comprises amino acid substitutions at one or more of K94, E95, G96, S97, F98, G99, V100b, D101, and P102; and LC-CDR3 comprises amino acid substitutions at one or more of Y89, S90, T91, D92, 193, S94, G95, N95a, H95b, W96, and V97.

23. The bispecific antibody of claim 10, wherein the heavy chain variable domain of the CD47-binding domain comprises a sequence with at least 90% identity to SEQ ID NO: 5, SEQ ID NO: 10, or SEQ ID NO: 13 and the light chain of the CD47-binding domain comprises a sequence with at least 90% identity to SEQ ID NO: 8, SEQ ID NO: 11, or SEQ ID NO: 14.

24. The bispecific antibody of claim 1, wherein the EpCAM-binding domain comprises three heavy chain (HC) complementarity determining regions (CDRs), HC-CDR1, HC-CDR2, and HC-CDR3 and three light chain (LC) CDRs, LC-CDR1, the LC-CDR2, and the LC-CDR3; wherein the CDRs are defined by an ordered set of sequences listed as HC-CDR1, HC-CDR2, HC-CDR3, LC-CDR1, LC-CDR2, LC-CDR3; and wherein the CDRs are selected from the group consisting of sequences having at least 90% identity to:
SEQ ID NO: 45, SEQ ID NO: 46, SEQ ID NO: 47, SEQ ID NO: 81, SEQ ID NO: 82, and SEQ ID NO: 83;
SEQ ID NO: 48, SEQ ID NO: 49, SEQ ID NO: 50, SEQ ID NO: 84, SEQ ID NO: 85, and SEQ ID NO: 86;
SEQ ID NO: 51, SEQ ID NO: 52, SEQ ID NO: 53, SEQ ID NO: 87, SEQ ID NO: 88, and SEQ ID NO: 89;
SEQ ID NO: 54, SEQ ID NO: 55, SEQ ID NO: 56, SEQ ID NO: 90, SEQ ID NO: 91, and SEQ ID NO: 92;
SEQ ID NO: 57, SEQ ID NO: 58, SEQ ID NO: 59, SEQ ID NO: 93, SEQ ID NO: 94, and SEQ ID NO: 95;
SEQ ID NO: 60, SEQ ID NO: 61, SEQ ID NO: 62, SEQ ID NO: 96, SEQ ID NO: 97, and SEQ ID NO: 98;
SEQ ID NO: 63, SEQ ID NO: 64, SEQ ID NO: 65, SEQ ID NO: 99, SEQ ID NO: 100, and SEQ ID NO: 101;
SEQ ID NO: 66, SEQ ID NO: 67, SEQ ID NO: 68, SEQ ID NO: 102, SEQ ID NO: 103, and SEQ ID NO: 104;
SEQ ID NO: 69, SEQ ID NO: 70, SEQ ID NO: 71, SEQ ID NO: 105, SEQ ID NO: 106, and SEQ ID NO: 107;
SEQ ID NO: 72, SEQ ID NO: 72, SEQ ID NO: 74, SEQ ID NO: 108, SEQ ID NO: 109, and SEQ ID NO: 110;
SEQ ID NO: 75, SEQ ID NO: 76, SEQ ID NO: 77, SEQ ID NO: 111, SEQ ID NO: 112, and SEQ ID NO: 113; and
SEQ ID NO: 78, SEQ ID NO: 79, SEQ ID NO: 80, SEQ ID NO: 114, SEQ ID NO: 115, and SEQ ID NO: 116.

25. The bispecific antibody of claim 1, wherein the EpCAM-binding domain comprises:
a sequence at least 90% identical to SEQ ID NO: 21 and
a sequence at least 90% identical to SEQ ID NO: 33;
a sequence at least 90% identical to SEQ ID NO: 22 and
a sequence at least 90% identical to SEQ ID NO: 34; or
a sequence at least 90% identical to SEQ ID NO: 24 and
a sequence at least 90% identical to SEQ ID NO: 36.

26. The bispecific antibody of claim 1, wherein less than 1 nM or less than 0.1 nM or less than 0.01 nM of the bispecific antibody increases a percentage of A431 cells engulfed by macrophage by at least 4-fold compared to a nonspecific IgG1 antibody control.

27. The bispecific antibody of claim 1, wherein a concentration of the antibody required to mediate antibody-dependent cellular phagocytosis of an EpCAM-positive, CD47-positive tumor cell by a macrophage is between 0.01 nM and −3 nM.

28. The bispecific antibody of claim 27, wherein the EpCAM-positive, CD47-positive tumor cell is an OVISE cell or an A431 cell.

29. The bispecific antibody of claim 27, wherein the EpCAM-positive, CD47-positive tumor cell is selected from a ductal pancreatic adenocarcinoma cell, an ovarian clear cell adenocarcinoma cell, a colon adenocarcinoma cell, a lung adenocarcinoma cell, an ovarian adenocarcinoma cell, a vulvar squamous cell carcinoma cell.

30. The bispecific antibody of claim 1, wherein 100 nM of the bispecific antibody inhibits the binding of SIRPα to CD47 on the surface of the tumor cell by at least 30%.

31. The bispecific antibody of claim 1, wherein the tumor cell is a CD47+ EpCAM+ tumor cell.

32. The bispecific antibody of claim 1, wherein the tumor cell expresses at least as many EpCAM proteins on its surface as an HCC-44 cell.

33. The bispecific antibody of claim 1, wherein the tumor cell expresses at least 100,000, at least 300,000, at least 600,000 or at least 1,500,000 EpCAM proteins on its surface.

34. The bispecific antibody of claim 1, wherein 400 nM of the bispecific antibody does not induce hemolysis of red blood cells in a hemagglutination assay.

35. A bispecific antibody comprising a CD47-binding domain and an EGFR-binding domain, wherein the bispecific antibody has a higher affinity for CD47 expressed on the surface of a tumor cell than for CD47 expressed on the surface of a red blood cell or a platelet.

36. The bispecific antibody of claim 35, further comprising an Fc domain.

37. The bispecific antibody of claim 35, wherein the tumor cell expresses EGFR.

38. The bispecific antibody of claim 35, wherein a concentration of the bispecific antibody required for half-maximal binding to a human red blood cell is greater than 500 nM.

39. The bispecific antibody of claim 35, wherein bispecific antibody binds to human CD47 with a KD of less than 100 nM as determined by surface plasmon resonance.

40. The bispecific antibody of claim 35, wherein the bispecific antibody binds to human CD47 with a KD of between 1 nM and 100 nM, between 1 nM and 50 nM, or between 5 nM and 50 nM as determined by surface plasmon resonance.

41. The bispecific antibody of claim 35, wherein the bispecific antibody binds to EGFR with a KD of less than 500 nM as determined by surface plasmon resonance.

42. The bispecific antibody of claim 35, wherein the bispecific antibody binds to EGFR with a KD of between 0.2 nM and 500 nM, between 0.2 nM and 200 nM, between 0.2 nM and 20 nM, between 0.2 nM and 2 nM, or between 2 nM and 10 nM as determined by surface plasmon resonance.

43. The bispecific antibody of claim 35, wherein the CD47-binding domain is a human or engineered human CD47-binding domain.

44. The bispecific antibody of claim 43, wherein the CD47-binding domain comprises a heavy chain variable domain and a light chain variable domain.

45. The bispecific antibody of claim 44, wherein the CD47-binding domain comprises an scFv.

46. The bispecific antibody of claim 35, wherein the EGFR-binding domain comprises a heavy chain variable domain and a light chain variable domain.

47. The bispecific antibody of claim 46, wherein the EGFR-binding domain comprises an scFv.

48. The bispecific antibody of claim 36, wherein the Fc domain is a human Fc domain.

49. The bispecific antibody of claim 36, wherein the isotype of the human Fc domain is IgG1, IgG2, or IgG4.

50. The bispecific antibody of claim 36, wherein the Fc domain is a heterodimeric Fc domain, wherein the heterodimeric Fc region comprises a knob chain and a hole chain, forming a knob-into-hole (KiH) structure.

51. The bispecific antibody of claim 50, wherein the knob chain comprises the mutation T366W and the hole chain comprises the mutations T366S, L368A, and Y407V, wherein amino acid position numbering is according to the EU index of Kabat et al.

52. The bispecific antibody of claim 50, wherein the bispecific antibody has an asymmetric three-chain knob-into-hole structure.

53. The bispecific antibody of claim 35, wherein the CD47-binding domain is an scFv.

54. The bispecific antibody of claim 35, wherein the EGFR-binding domain is an scFv.

55. The bispecific antibody of claim 35, wherein the CD47-binding domain comprises three heavy chain (HC) complementarity determining regions (CDRs): HC-CDR1, HC-CDR2, and HC-CDR3; and three light chain (LC) CDRs: LC-CDR1, the LC-CDR2, and the LC-CDR3; wherein HC-CDR1 comprises SEQ ID NO: 15, HC-CDR2 comprises SEQ ID NO: 16, HC-CDR3 comprises a sequence with at least 50% identity to SEQ ID NO: 17, LC-CDR1 comprises SEQ ID NO: 18, LC-CDR2 comprises SEQ ID NO: 19, and LC-CDR3 comprises a sequence with at least 50% identity to SEQ ID NO: 20.

56. The bispecific antibody of claim 55, wherein HC-CDR3 comprises amino acid substitutions at one or more of K94, E95, G96, S97, F98, G99, V100b, D101, and P102; and LC-CDR3 comprises amino acid substitutions at one or more of Y89, S90, T91, D92, 193, S94, G95, N95a, H95b, W96, and V97.

57. The bispecific antibody of claim 44, wherein the heavy chain variable domain of the CD47-binding domain comprises a sequence with at least 90% identity to SEQ ID NO: 5, SEQ ID NO: 10, or SEQ ID NO: 13 and the light chain of the CD47-binding domain comprises a sequence with at least 90% identity to SEQ ID NO: 8, SEQ ID NO: 11, or SEQ ID NO: 14.

58. The bispecific antibody of claim 35, wherein the EGFR-binding domain comprises three heavy chain (HC) complementarity determining regions (CDRs), HC-CDR1, HC-CDR2, and HC-CDR3 and three light chain (LC) CDRs, LC-CDR1, the LC-CDR2, and the LC-CDR3; wherein the CDRs are defined by an ordered set of sequences listed as HC-CDR1, HC-CDR2, HC-CDR3, LC-CDR1, LC-CDR2, LC-CDR3; and wherein the CDRs are selected from the group consisting of sequences having at least 90% identity to:
SEQ ID NO: 121, SEQ ID NO: 122, SEQ ID NO: 123, SEQ ID NO: 124, SEQ ID NO: 125, and SEQ ID NO: 126.

59. The bispecific antibody of claim 35, wherein the EGFR-binding domain comprises:
a sequence at least 90% identical to SEQ ID NO: 127 and
a sequence at least 90% identical to SEQ ID NO: 128.

60. The bispecific antibody of claim 35, wherein less than 1 nM or less than 0.01 nM of the bispecific antibody increases a percentage of A431 cells engulfed by macrophage by at least 4-fold compared to a nonspecific IgG1 antibody control.

61. The bispecific antibody of claim 35, wherein a concentration of the bispecific antibody required to mediate antibody-dependent cellular phagocytosis of an EGFR-positive, CD47-positive tumor cell by a macrophage is between 0.01 nM and −3 nM.

62. The bispecific antibody of claim 61, wherein the EGFR-positive, CD47-positive tumor cell is an OVISE cell or an A431 cell.

63. The bispecific antibody of claim 61, wherein the EGFR-positive, CD47-positive tumor cell is selected from an epidermoid carcinoma, colorectal adenocarcinoma, pancreas adenocarcinoma, lung squamous cell carcinoma, or gastric carcinoma.

64. The bispecific antibody of claim 35, wherein 100 nM of the bispecific antibody inhibits the binding of SIRPα to CD47 on the surface of a cell by at least 30%.

65. The bispecific antibody of claim 64, wherein the cell is a CD47+ EGFR+ tumor cell.

66. The bispecific antibody of claim 65, wherein the cell expresses at least as many EGFR proteins on its surface as a LS1034 cell.

67. The bispecific antibody of claim 65, wherein the cell expresses at least 50,000, at least 100,000, at least 300,000, at least 600,000 or at least 1,500,000 EGFR proteins on its surface.

68. The bispecific antibody of claim 35, wherein 400 nM of the bispecific antibody does not induce hemolysis of red blood cells in a hemagglutination assay.
